(19) United States
(12) Patent Application Publication
(10) Pub. No.: US 2024/0018189 A1
(43) Pub. Date: Jan. 18, 2024

(54) CONJUGATED HEPCIDIN MIMETICS

(71) Applicant: Protagonist Therapeutics, Inc., Newark, CA (US)

(72) Inventors: Gregory Thomas BOURNE, Moggill, Brisbane, Queensland (AU); Ashok BHANDARI, Pleasanton, CA (US); Jie ZHANG, Salisbury (AU); Brian Troy FREDERICK, Ben Lomond, CA (US); Mark Leslie SMYTHE, Bardon, Queensland (AU)

(73) Assignee: Protagonist Therapeutics, Inc., Newark, CA (US)

(21) Appl. No.: 18/018,482

(22) PCT Filed: Jul. 28, 2021
(86) PCT No.: PCT/US21/43584

§ 371 Date: Jan. 27, 2023

Related U.S. Application Data
(60) Provisional application No. 63/057,574, filed on Jul. 28, 2020.
(60) Provisional application No. 63/057,577, filed on Jul. 28, 2020.
(60) Provisional application No. 63/057,582, filed on Jul. 28, 2020.
(60) Provisional application No. 63/057,583, filed on Jul. 28, 2020.
(60) Provisional application No. 63/169,515, filed on Apr. 1, 2021.
(60) Provisional application No. 63/169,527, filed on Apr. 1, 2021.
(60) Provisional application No. 63/169,533, filed on Apr. 1, 2021.

(51) Int. Cl. C07K 7/64; A61K 47/64; A61P 7/00

(52) U.S. Cl. CPC C07K 7/64; A61K 38/00; A61P 7/00; A61K 47/64

(57) ABSTRACT

The present invention provides hepcidin analogues with improved in vivo half lives, and related pharmaceutical compositions and methods of use thereof.

### CROSS REFERENCE TO RELATED APPLICATIONS

This application is a National Stage application under 35 U.S.C. § 371 of International Application No. PCT/US2021/043582, filed Jul. 28, 2021, which claims the priority benefit of U.S. provisional patent application No. 63/057,582, which was filed on Jul. 28, 2020; U.S. provisional patent application No. 63/057,577, which was filed on Jul. 28, 2020; and U.S. provisional patent application No. 63/169,527, which was filed on Apr. 1, 2021, U.S. provisional patent application No. 63/169,533, which was filed on Apr. 1, 2021; U.S. provisional patent application No. 63/169,515, which was filed on Apr. 1, 2021; U.S. provisional patent application No. 63/057,583, which was filed on Jul. 28, 2020; U.S. provisional patent application No. 63/057,574, which was filed on Jul. 28, 2020; the disclosure of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 28, 2021, is named PRTH_058_02WO_ST25.txt and is 268 KB in size.

### FIELD OF THE INVENTION

The present invention relates, inter alia, to certain hepcidin peptide analogues, including both peptide monomers and peptide dimers, and conjugates and derivatives thereof, as well as compositions comprising the peptide analogues, and to the use of the peptide analogues in the treatment and/or prevention of a variety of diseases, conditions or disorders, including treatment and/or prevention of iron overload diseases such as hereditary hemochromatosis, iron-loading anemias, and other conditions and disorders described herein.

### BACKGROUND

Hepcidin (also referred to as LEAP-1), a peptide hormone produced by the liver, is a regulator of iron homeostasis in humans and other mammals. Hepcidin acts by binding to its receptor, the iron export channel ferroportin, causing its internalization and degradation. Human hepcidin is a 25-amino acid peptide (Hep25). See Krause et al. (2000) FEBS Lett 480:147-150, and Park et al. (2001) J Biol Chem 276:7806-7810. The structure of the bioactive 25-amino acid form of hepcidin is a simple hairpin with 8 cysteines that form 4 disulfide bonds as described by Jordan et al. J Biol Chem 284:24155-67. The N terminal region is required for iron-regulatory function, and deletion of 5 N-terminal amino acid residues results in a loss of iron-regulatory function. See Nemeth et al. (2006) Blood 107:328-33.

Abnormal hepcidin activity is associated with iron overload diseases, including hereditary hemochromatosis (HH) and iron-loading anemias. Hereditary hemochromatosis is a genetic iron overload disease that is mainly caused by hepcidin deficiency or in some cases by hepcidin resistance. This allows excessive absorption of iron from the diet and development of iron overload. Clinical manifestations of HH may include liver disease (e.g., hepatic cirrhosis and hepatocellular carcinoma), diabetes, and heart failure. Currently, the only treatment for HH is regular phlebotomy, which is very burdensome for the patients. Iron-loading anemias are hereditary anemias with ineffective erythropoiesis such as β-thalassemia, which are accompanied by severe iron overload. Complications from iron overload are the main causes of morbidity and mortality for these patients. Hepcidin deficiency is the main cause of iron overload in non-transfused patients, and contributes to iron overload in transfused patients. The current treatment for iron overload in these patients is iron chelation which is very burdensome, sometimes ineffective, and accompanied by frequent side effects.

Hepcidin has a number of limitations that restrict its use as a drug, including a difficult synthetic process due in part to aggregation and precipitation of the protein during folding, which in turn leads to high cost of goods. What are needed in the art are compounds having hepcidin activity and also possessing other beneficial physical properties such as improved solubility, stability, and/or potency, so that hepcidin-like compounds might be produced affordably and used to treat hepcidin-related diseases and disorders such as, e.g., those described herein.

The present invention addresses such needs, providing novel peptide analogues, including both peptide monomer analogues and peptide dimer analogues, having hepcidin activity and also having other beneficial properties making the peptides of the present invention suitable alternatives to hepcidin.

### BRIEF SUMMARY OF THE INVENTION

The present invention generally relates to peptide analogues, including both monomer and dimers, exhibiting hepcidin activity and methods of using the same.

In one aspect, the present invention includes a hepcidin analogue comprising a peptide of Formula (W-I):

R¹-X1-Thr-His-X4-B2-X6-X7-Xaa1-X9-Xaa2-J-Y1-Y2-R² (W-I)
or a peptide dimer comprising two peptides according to Formula W-I;
or a pharmaceutically acceptable salt, or a solvate thereof,
wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is —NH₂ or —OH;
X1 is Asp, (D)Asp, or substituted Asp;
X4 is F, NMePhe, or Dpa;
Xaa1 is B5; and
   i) B5 is absent, Lys, D-Lys, Spiro_pip, Arg(nitro), Arg(dimethyl), Cit, Pro(4-amino), or Cav; and Xaa2 is B7(L1Z); and B7 is Lys, or D-Lys;
or
   ii) Xaa1 is B5(L1Z); B5 is Lys, D-Lys, NMeLys, Dap or Dap-Dap; and Xaa2 is B7; and B7 is Glu, NMeGlu, or absent;
B2 is Pro, hydroxyPro, hydroxy(D)Pro, or NPC;
X6 is Cys, NMeCys, SMeCys, or aMeCys;
X7 is Ile, Leu, or NMeLeu;
X9 is Phe, bhPhe, or NMePhe;
L1 is absent, Dap, D-Dap, or isoGlu, PEG, Ahx, isoGlu-PEG, PEG-isoGlu, PEG-Ahx, isoGlu-Ahx, isoGlu-PEG-Ahx, or PEG-pep;
Ahx is an aminohexanoic acid moiety; pep is a peptide consisting of one or more amino acids; PEG is —[C(O)—CH₂-(Peg)ₙ-N(H)]ₘ—, or —[C(O)—CH₂—CH₂-(Peg)ₙ-N(H)]ₘ—; and Peg is —OCH₂CH₂—, m is 1, 2, or 3; and n is an integer between 1-100K;
Z is a half-life extension moiety;
J is absent, any amino acid, or a peptide chain consisting of 1-5 amino acids, wherein each amino acid is independently selected from Pro, (D)Pro, hydroxyPro, hydroxy(D)Pro, Arg, MeArg, Lys, (D)Lys, Lys(Ac), (D)Lys(Ac), Ser, MeSer, Sar, and Gly;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
hydroxyPro is Proline substituted with hydroxy, hydroxy(D)Pro is (D)Proline substituted with hydroxy,
wherein
   i) the peptide of formula W-I is optionally PEGylated on one or more R¹, B5, B6, B7, J, Y2, or R²; and
   ii) the peptide is optionally cyclized via a disulfide bond between X6 and YT;
      or the hepcidin analogue is any one of peptides listed in Table 3A or Table 3B, and wherein the peptide is optionally cyclized via a disulfide bond between X6 and Y1

In one aspect, the present invention includes a hepcidin analogue comprising a peptide of Formula (I):

R¹-Asp-Thr-His-Dpa-B2-Cys-Ile-Xaa1-bhF-Xaa2-J-Y1-Y2-R² (I)
or a peptide dimer comprising two peptides according to Formula I, or a pharmaceutically acceptable salt, or a solvate thereof,
wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is NH₂ or OH;
Xaa1 is B5; and
   i) B5 is absent, Lys, D-Lys, Spiro_pip, Arg(nitro), Arg(dimethyl), Cit, Pro(4-amino), or Cav; and Xaa2 is B7(L1Z); and B7 is Lys, or D-Lys;
or
   ii) Xaa1 is B5(L1Z); B5 is Lys, D-Lys, Dap or Dap-Dap; and Xaa2 is B7; and B7 is Glu, D-Lys, or absent;
B2 is Pro, or NPC;
B7 is Lys, or D-Lys;
L1 is absent, Dap, D-Dap, or isoGlu, PEG, Ahx, isoGlu-PEG, PEG-isoGlu, PEG-Ahx, isoGlu-Ahx, isoGlu-PEG-Ahx, or PEG-pep; Ahx is an aminohexanoic acid moiety; pep is a peptide consisting of one or more amino acids; PEG is —[C(O)—CH₂-(Peg)ₙ-N(H)]ₘ—, or —[C(O)—CH₂—CH₂-(Peg)ₙ-N(H)]ₘ—; and Peg is —OCH₂CH₂—, m is 1, 2, or 3; and n is an integer between 1-100K;
Z is a half-life extension moiety;
J is Arg, Lys, D-Lys, Spiro_pip, Arg(nitro), Arg(dimethyl), Cit, Pro(4-amino), Cav, Pro, -Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly-(SEQ ID NO:251), or absent; or J is any amino acid;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
wherein
   i) the peptide of formula I is optionally PEGylated on one or more R¹, B5, B6, B7, J, Y2, or R²; and
      ii) the peptide is optionally cyclized via a disulfide bond between Cys and Y1.

In one embodiment, Xaa1 is B5; B5 is absent, Lys, or D-Lys; and Xaa2 is B7(L1Z); and B7 is Lys, or D-Lys; and L1 is attached to N^{ε} of Lys, D-Lys, homoLys, or N^{β} of Dapa.

In another embodiment, Xaa1 is B5(L1Z); B5 is Lys, or D-Lys; and Xaa2 is B7; and B7 is Glu or absent; and L1 is attached to N^{ε} of Lys.

In particular embodiments of hepcidin analogues disclosed herein, the half-life extending moiety is C₁₀-C₂₁ alkanoyl.

In one particular embodiment, B7 is Lys, D-Lys, homoLys, or a-Me-Lys.

In particular embodiments of any of the hepcidin analogues or dimers of the present invention, the half-life extension moiety is selected from C12 (Lauric acid), C14 (Mysteric acid), C16(Palmitic acid), C18 (Stearic acid), C20, C12 diacid, C14 diacid, C16 diacid, C18 diacid, C20 diacid, biotin, and isovaleric acid. In certain embodiments, the half-life extension moiety is attached to a linker moiety that is attached to the peptide. In certain embodiments, the half-life extension moiety increases the molecular weight of the hepcidin analogue by about 50 D to about 2 KD. In various embodiments, the half-life extension moiety increases serum half-life, enhances solubility, and/or improves bioavailability of the hepcidin analogue.

In certain embodiments, a peptide analogue or dimer of the present invention comprises an isovaleric acid moiety conjugated to an N-terminal Asp residue.

In certain embodiments, a peptide analogue of the present invention comprises an amidated C-terminal residue.

In certain embodiments, the present invention provides hepcidin analogues, including any hepcidin analogue or peptide disclosed herein or comprising or consisting of a sequence or structure disclosed herein, including but not limited to wherein the hepcidin analogue or peptide comprises a disulfide bond between two Cys residues.

In certain embodiments, a hepcidin analogue or dimer of the present invention comprises the sequence: Asp-Thr-His-Phe-Pro-Cys-Ile-Lys-Phe-Glu-Pro-Arg-Ser-Lys-Gly-Cys-Lys (SEQ ID NO:252), or comprises a sequence having at least 80%, at least 90%, or at least 94% identity to this sequence.

In certain embodiments, a hepcidin analogue or dimer of the present invention comprises the sequence: Asp-Thr-His-Phe-Pro-Cys-Ile-Lys-Phe-Lys-Pro-Arg-Ser-Lys-Gly-Cys-Lys (SEQ ID NO:1), or comprises a sequence having at least 80%, at least 90%, or at least 94% identity to this sequence.

In a related embodiment, the present invention includes a polynucleotide that encodes a peptide of a hepcidin analogue or dimer (or monomer subunit of a dimer) of the present invention.

In a further related embodiment, the present invention includes a vector comprising a polynucleotide of the invention. In particular embodiments, the vector is an expression vector comprising a promoter operably linked to the polynucleotide, e.g., in a manner that promotes expression of the polynucleotide.

In another embodiment, the present invention includes a pharmaceutical composition, comprising a hepcidin analogue, dimer, polynucleotide, or vector of the present invention, and a pharmaceutically acceptable carrier, excipient or vehicle.

In another embodiments, the present invention provides a method of binding a ferroportin or inducing ferroportin internalization and degradation, comprising contacting the ferroportin with at least one hepcidin analogue, dimer or composition of the present invention.

In a further embodiment, the present invention includes a method for treating a disease of iron metabolism in a subject in need thereof comprising providing to the subject an effective amount of a pharmaceutical composition of the present invention. In certain embodiments, the pharmaceutical composition is provided to the subject by an oral, intravenous, peritoneal, intradermal, subcutaneous, intramuscular, intrathecal, inhalation, vaporization, nebulization, sublingual, buccal, parenteral, rectal, vaginal, or topical route of administration. In certain embodiments, the pharmaceutical composition is provided to the subject by an oral or subcutaneous route of administration. In certain embodiments, the disease of iron metabolism is an iron overload disease. In certain embodiments, the pharmaceutical composition is provided to the subject at most or about twice daily, at most or about once daily, at most or about once every two days, at most or about once a week, or at most or about once a month.

In particular embodiments, the hepcidin analogue is provided to the subject at a dosage of about 1 mg to about 100 mg or about 1 mg to about 5 mg.

In another embodiment, the present invention provides a device comprising pharmaceutical composition of the present invention, for delivery of a hepcidin analogue or dimer of the invention to a subject, optionally orally or subcutaneously.

In yet another embodiment, the present invention includes a kit comprising a pharmaceutical composition of the invention, packaged with a reagent, a device, or an instructional material, or a combination thereof.

### DETAILED DESCRIPTION OF THE INVENTION

The present invention relates generally to hepcidin analogue peptides and methods of making and using the same. In certain embodiments, the hepcidin analogues exhibit one or more hepcidin activity. In certain embodiments, the present invention relates to hepcidin peptide analogues comprising one or more peptide subunit that forms a cyclized structures through an intramolecular bond, e.g., an intramolecular disulfide bond. In particular embodiments, the cyclized structure has increased potency and selectivity as compared to non-cyclized hepcidin peptides and analogies thereof. In particular embodiments, hepcidin analogue peptides of the present invention exhibit increased half-lives, e.g., when delivered orally, as compared to hepcidin or previous hepcidin analogues.

### Definitions and Nomenclature

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). The term “mammal” refers to any mammalian species such as a human, mouse, rat, dog, cat, hamster, guinea pig, rabbit, livestock, and the like.

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The term “peptide analogue” or “hepcidin anloguuue” as used herein, refers broadly to peptide monomers and peptide dimers comprising one or more structural features and/or functional activities in common with hepcidin, or a functional region thereof. In certain embodiments, a peptide analogue includes peptides sharing substantial amino acid sequence identity with hepcidin, e.g., peptides that comprise one or more amino acid insertions, deletions, or substitutions as compared to a wild-type hepcidin, e.g., human hepcidin, amino acid sequence. In certain embodiments, a peptide analogue comprises one or more additional modification, such as, e.g., conjugation to another compound. Encompassed by the term “peptide analogue” is any peptide monomer or peptide dimer of the present invention. In certain instances, a “peptide analog” may also or alternatively be referred to herein as a “hepcidin analogue,” “hepcidin peptide analogue,” or a “hepcidin analogue peptide.”

The recitations “sequence identity”, “percent identity”, “percent homology”, or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids and aromatic amino acids. See, for example, the table below. In some embodiments of the invention, one or more Met residues are substituted with norleucine (Nle) which is a bioisostere for Met, but which, as opposed to Met, is not readily oxidized. In some embodiments, one or more Trp residues are substituted with Phe, or one or more Phe residues are substituted with Trp, while in some embodiments, one or more Pro residues are substituted with Npc, or one or more Npc residues are substituted with Pro. Another example of a conservative substitution with a residue normally not found in endogenous, mammalian peptides and proteins is the conservative substitution of Arg or Lys with, for example, ornithine, canavanine, aminoethylcysteine or another basic amino acid. In some embodiments, another conservative substitution is the substitution of one or more Pro residues with bhPro or Leu or D-Npc (isonipecotic acid). For further information concerning phenotypically silent substitutions in peptides and proteins, see, for example, Bowie et. al. Science 247, 1306-1310, 1990. In the scheme below, conservative substitutions of amino acids are grouped by physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The 20 “standard,” natural amino acids are listed in the above tables. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-”moiety at the N-terminus of the sequence in question indicates a hydrogen atom, corresponding to the presence of a free primary or secondary amino group at the N-terminus, while an “—OH” or an “—NH₂” moiety at the C-terminus of the sequence indicates a hydroxy group or an amino group, corresponding to the presence of an amido (CONH₂) group at the C-terminus, respectively. In each sequence of the invention, a C-terminal “—OH” moiety may be substituted for a C-terminal “—NH₂” moiety, and vice-versa. It is further understood that the moiety at the amino terminus or carboxy terminus may be a bond, e.g., a covalent bond, particularly in situations where the amino terminus or carboxy terminus is bound to a linker or to another chemical moiety, e.g., a PEG moiety.

The term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the C- or N-terminus of a polypeptide.

The term “carboxy,” as used herein, refers to —CO₂H.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1A and 1B.

TABLE 1A
Abbreviations of Non-Natural Amino Acids and Chemical Moieties

| Abbreviation | Definition |
|---|---|
| bh, b-h, bhomo, or b-homo | β-homo |
| DIG | Diglycolic acid |
| Dapa or Dap | Diaminopropionic acid |
| Daba or Dab | Diaminobutyric acid |
| Pen | Penicillamine |
| Sarc or Sar | Sarcosine |
| Cit | Citrulline |
| Cav | Cavanine |
| NMe-Arg | N-Methyl-Arginine |
| NMe-Trp | N-Methyl-Tryptophan |
| NMe-Phe | N-Methyl-Phenylalanine |
| Ac- | Acetyl |
| 2-Nal | 2-Napthylalanine |
| 1-Nal | 1-Napthylalanine |
| Bip | Biphenylalanine |
| 2Pal | 2-Pyridylalanine |
| βAla or bAla | beta-Alanine |
| Aib | 2-aminoisobutyric acid |
| Azt | azetidine-2-carboxylic acid |
| Tic | L-1,2,3,4-Tetrahydroisoquinoline- 3-carboxylic acid |
| Phe(OMe) or Tyr(Me) | Tyrosine (4-Methyl) |
| N-MeLys or (Me)Lys | N-Methyl-Lysine |
| Dpa or DIP | β,β-diphenylalanine |
| NH₂ | Free Amine |
| CONH₂ | Amide |
| COOH | Acid |
| Phe(4-F), Phe(4F), (4-F)Phe or (4F)Phe | 4-Fluoro-L-Phenylalanine |
| Phe(4-CF₃), Phe(4CF₃), (4-CF₃)Phe or | (4-Trifluoromethyl)-L-Phenylalanine |
| (4CF₃)Phe | |
| Phe(2,3,5-triF), or (2,3,5-triF)Phe | (2,3,5-Trifluoro)-L-Phenylalanine |
| Palm | Palmitoic or Palmitoyl or C(O)—(CH₂)₁₄CH₃ |
| (Peg)n | —(OCH₂CH₂)ₙ— n is 1, 2, 3, 4, etc |
| Peg2 | —(OCH₂CH₂)₂— |
| Peg4 | —(OCH₂CH₂)₄— |
| Peg8 | —(OCH₂CH₂)₈— |
| Peg11 | —(OCH₂CH₂)₁₁— |
| Peg12 | —(OCH₂CH₂)₁₂— |
| 1Peg2 or 1PEG2 | —[C(O)—CH₂—(Peg)₂—NH]— or —[C(O)—CH₂—(OCH₂CH₂)₂—NH]— |
| 1Peg2-1Peg2 or 1PEG2-1PEG2 | —[(C(O)—CH₂—(OCH₂CH₂)₂—NH-C(O)—CH₂—(OCH₂CH₂)₂—NH-]— |
| 2Peg2 or PEG2 | —[C(O)—CH₂—CH₂—(Peg)₂—NH]— or —[C(O)—CH₂—CH₂—(OCH₂CH₂)₂—NH]— |
| 2Peg4 or PEG4 | —[C(O)—CH₂—CH₂—(Peg)₄—NH]— or —[C(O)—CH₂—CH₂—(OCH₂CH₂)₄—NH]— |
| 1Peg8 or 1PEG8 | —[C(O)—CH₂—(Peg)₈—NH]— or —[C(O)—CH₂—(OCH₂CH₂)₈—NH]— |
| 2Peg8 or PEG8 | —[C(O)—CH₂—CH₂—(Peg)₈—NH]— or —[C(O)—CH₂—CH₂—(OCH₂CH₂)₈—NH]— |
| 1Peg11 or 1PEG11 | —[C(O)—CH₂—(Peg)₁₁—NH]— or —[C(O)—CH₂—(OCH₂CH₂)₁₁—NH]— |
| 2Peg11 or PEG11 | —[C(O)—CH₂—CH₂—(Peg)₁₁—NH]— or —[C(O)—CH₂—CH₂—(OCH₂CH₂)₁₁—NH]— |
| 2Peg11′ or 2Peg12 or PEG12 | —[C(O)—CH₂—CH₂—(Peg)₁₂—NH]— or —[C(O)—CH₂—CH₂—(OCH₂CH₂)₁₂—NH]— |
| 2Peg11′ Palm or | —[C(O)—CH₂—CH₂—(Peg)₁₂—NH]— C(O)—(CH₂)₁₄CH₃ or —[C(O)—CH₂—CH₂ |
| 2Peg12_Palm or PEG12_Palm | —[OCH₂CH₂)₁₂—NH]— C(O)—(CH₂)₁₄CH₃ |
| 2Peg11′ _C18_Diacid or | —[C(O)—CH₂—CH₂—(Peg)₁₂—NH]-C(O)—(CH₂)₁₆C(O)OH or —[C(O)—CH₂—CH2-(OCH₂CH₂)₁₂—NH]-C(O)—(CH₂)₁₆C(O)OH |
| 2Peg12_C18_Diacid | |
| 2Peg11′ Ahx_Palm or | —[C(O)—CH₂—CH₂—(Peg)₁₂—NH]— C(O)—(CH₂)₅—N(H)-C(O)—(CH₂)₁₄CH3 or —[C(O)—CH₂—CH₂—(OCH₂CH₂)₁₂—NH]—C(O)—(CH₂)₅—N(H)— |
| 2Peg12_Ahx_Palm or | C(O)—(CH₂)₁₄CH₃ |
| PEG12_Ahx_Palm | |
| Lys(2Peg11′ Palm) or Lys(PEG11′_Palm) or Lys(PEG12_Palm) | 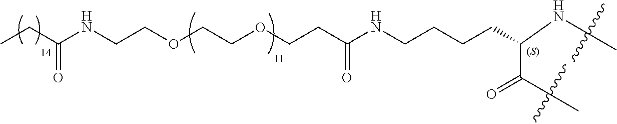 |
| Lys(2Peg11′C18_Diacid) or Lys(PEG12_C18_Diacid) | 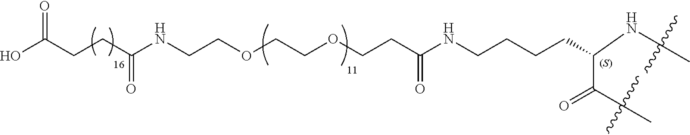 |
| Lys(2Peg1l′ Ahx_Palm) or Lys(PEG12_Ahx_Palm) | 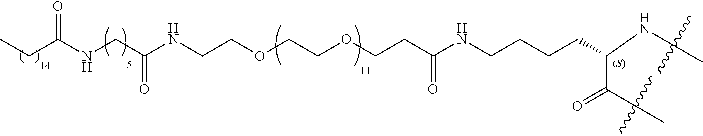 |
| Lys(2Peg1l′ IsoGlu_Palm) or Lys(PEG12_IsoGlu Palm | 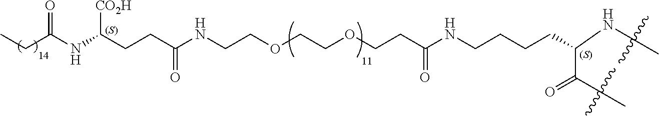 |
| Lys(2Peg1l′ Ahx_C18_Diacid) or Lys(PEG12_Ahx_C18_Diacid) | 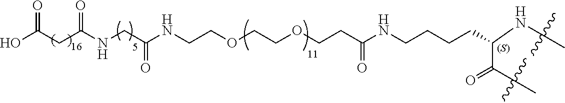 |
| Lys(2Peg11′ Ahx_IsoGlu_C18_Diacid) or Lys(PEG12_Ahx_IsoGlu C18_Diacid) | 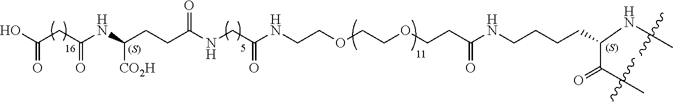 |
| Lys(2Peg1l′ Ahx IsoGlu_Behenic_acid) or Lys(PEG12_Ahx_IsoGlu_Behenic_acid) | 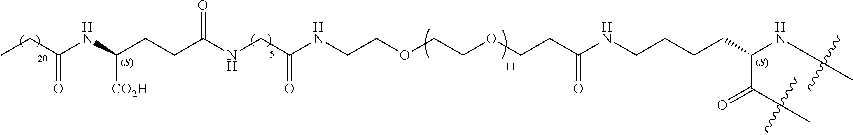 |
| IsoGlu_Palm | 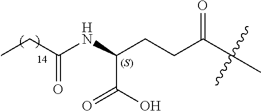 |
| Lys(IsoGlu_Palm) or Z minus | 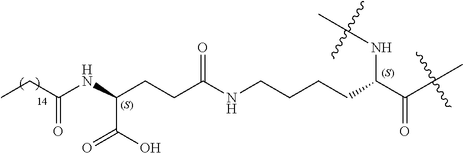 |
| Lys(Ahx_Palm) | 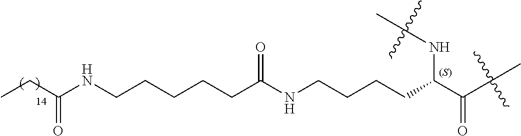 |
| Lys(1Peg2_1Peg2_Ahx_C18_Diacid) or Lys(1PEG2_1PEG2 Ahx C18 Diacid) | 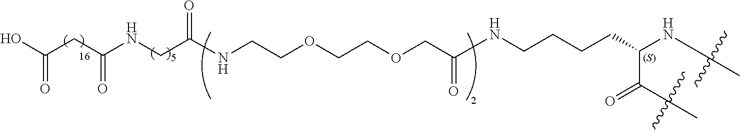 |
| Lys(1Peg2_1Peg2_IsoGluC18_Diacid) or Lys(1PEG2_1PEG2_IsoGluC18_Diacid) | 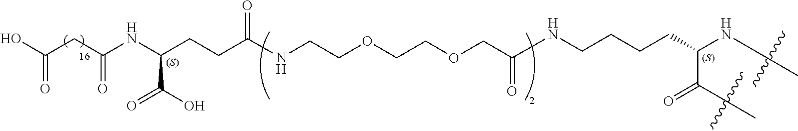 |
| (D)Lys(Peg11_OMe) or (D)Lys(PEG11_OMe) | 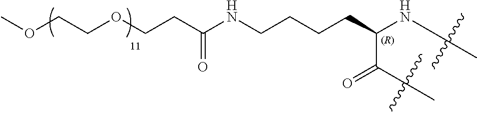 |
| Peg11_OMe or PEG11_OMe | —[C(O)—CH₂—CH₂—(OCH₂CH₂)11-OMe] |
| Lys(Ahx_Ahx C18 Diacid) | 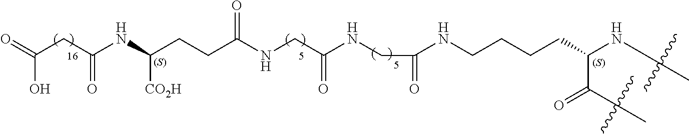 |
| Lys(Ado_C18_Diacid) | 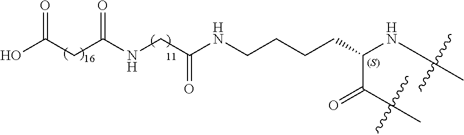 |
| Lys(Ado_Palm) | 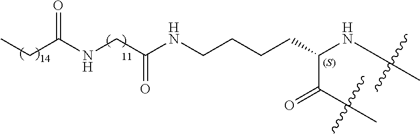 |
| Lys(Ado_IsoGlu C18_Diacid) | 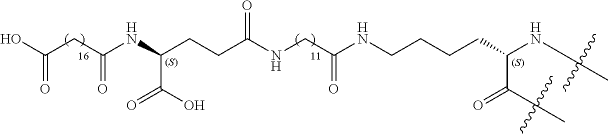 |
| Lys(2Peg11′ 2Peg11′_Palm) or Lys(PEG12_PEG12_Palm) | 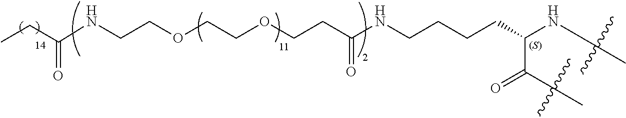 |
| Lys(2Peg4_Palm) or Lys(PEG4_Palm) | 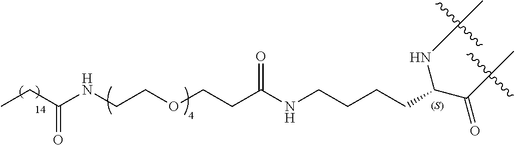 |
| Lys(2Peg4_Ahx_Palm) or Lys(PEG4_Ahx_Palm) | 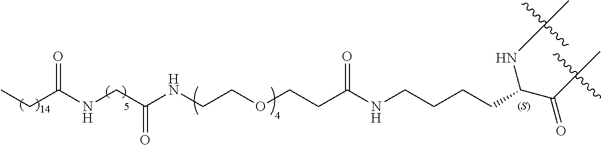 |
| Lys(Ac) | 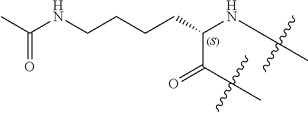 |
| Lys(Ahx) | 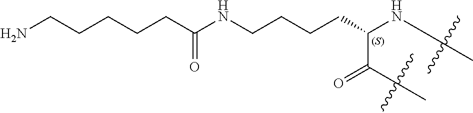 |
| Lys(Ahx_PEG20K) | 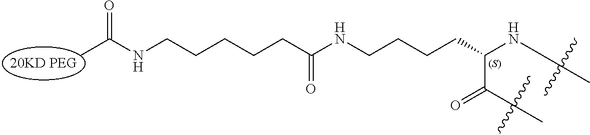 |
| Lys([Lys(2Peg11′ Palm)2 or Lys([Lys(PEG12_Palm)2 | 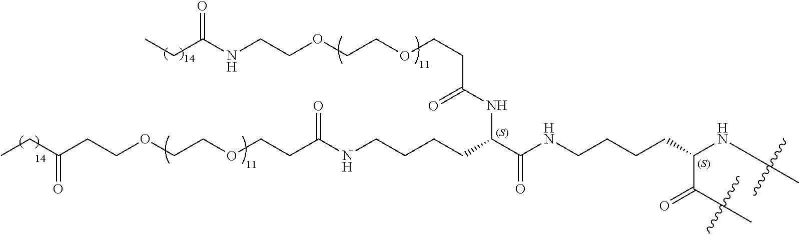 |
| Lys(1Peg2_Ahx_Palm) or Lys(1PEG2_Ahx_Palm) | 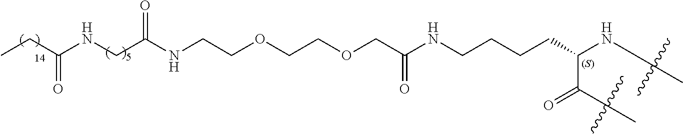 |
| Lys(1Peg2_Ahx_C18_Diacid) or Lys(1PEG2_Ahx_C18_Diacid) | 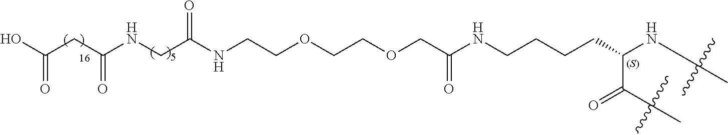 |
| Lys(2Peg8_Ahx_Palm) or Lys(PEG8_Ahx_Palm) | 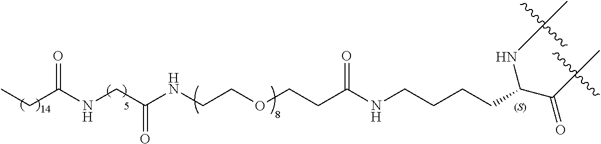 |
| Lys(2Peg8_Ahx C18_Diacid) or Lys(PEG8_Ahx_C18_Diacid) | 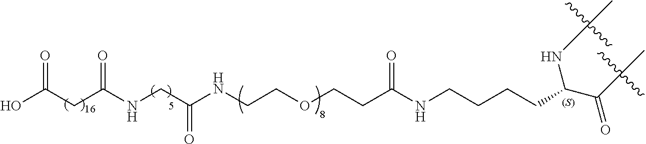 |
| Lys(1Peg2_1Peg2_Ahx_Palm) or Lys(1PEG2_1PEG2 Ahx Palm) | 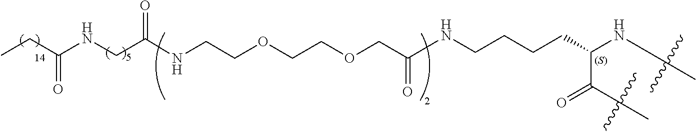 |
| Lys(2Peg11′ AlbuTag) or Lys(PEG12_AlbuTag) | 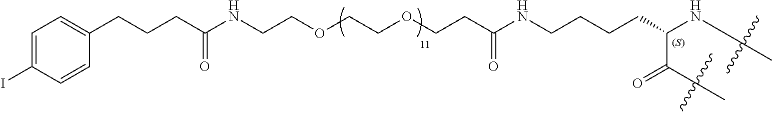 |
| (D)Lys_IVA | 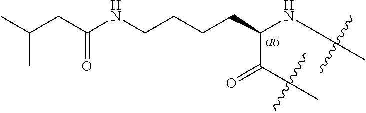 |
| Lys(2Peg11′ IsoGlu_C14_Diacid) or Lys(PEG12_IsoGlu_C14_Diacid) | 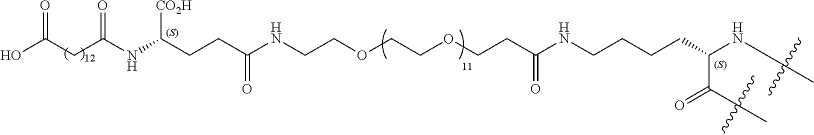 |
| Lys(2Peg1l′ IsoGlu_C16_Diacid) or Lys(PEG12_IsoGlu_C16_Diacid) | 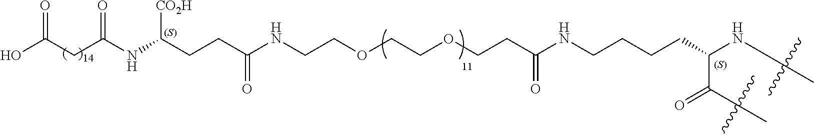 |
| Lys(2Peg11′ IsoGlu C18_Diacid) or Lys(PEG12_IsoGlu_C18_Diacid) | 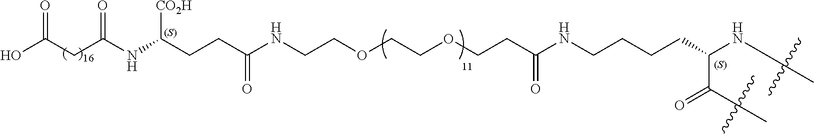 |
| Lys(2Peg1l′ IsoGlu_C20_Diacid) or Lys(PEG12_IsoGlu C20_Diacid) | 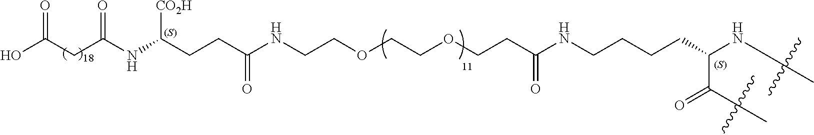 |
| Peg13 | Bifunctional PEG linker with 13 PolyEthylene Glycol units |
| Peg25 | Bifunctional PEG linker with 25 PolyEthylene Glycol units |
| Peg1K | Bifunctional PEG linker with Poly Ethylene Glycol Mol wt of 1000Da |
| Peg2K | Bifunctional PEG linker with Poly Ethylene Glycol Mol wt of 2000Da |
| Peg3.4K | Bifunctional PEG linker with PolyEthylene Glycol Mol wt of 3400Da |
| Peg5K | Bifunctional PEG linker with Poly Ethylene Glycol Mol wt of 5000Da |
| IDA or Ida | Iminodiacetic acid |
| IDA-Palm | (Palmityl)-Iminodiacetic acid |
| hPhe | homoPhenylalanine |
| Ahx | Aminohexanoic acid |
| Isovaleric Acid | 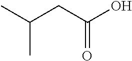 |
| DIG-OH | Glycolic monoacid |
| Triazine | Amino propyl Triazine di-acid |
| Boc-Triazine | Boc-Triazine di-acid |
| Trifluorobutyric acid | 4,4,4-Trifluorobutyric acid |
| 2-Methyltrifluorobutyric acid | 2-methyl-4,4,4-Butyric acid |
| Trifluoropentanoic acid | 5,5,5-Trifluoropentanoic acid |
| 1,4- Phenylenediacetic acid | para-Phenylenediacetic acid |
| 1,3-Phenylenediacetic acid | meta-Phenylenediacetic acid |
| DTT | Dithiothreotol |
| BβTrp or bhTrp | β-homo Tryptophane |
| BβPhe or bhPhe | β-homophenylalanine |
| Phe(4-CF₃) | 4-TrifluoromethylPhenylalanine |
| βGlu or bGlu | β-Glutamic acid |
| Asp_OMe or (OMe)Asp | 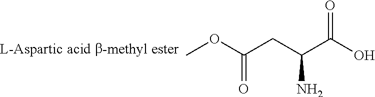 |
| Glu_OMe or (OMe)Glu | 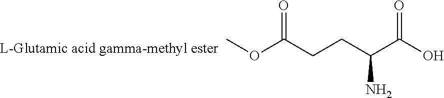 |
| βhGlu or bhGlu | β-homoglutamic acid |
| 2-2-Indane | 2-Aminoindane-2-carboxylic acid |
| 1-1-Indane | 1-Aminoindane-1-carboxylic acid |
| hCha | homocyclohexylalanine |
| Cyclobutyl | Cyclobutylalanine |
| hLeu | Homoleucine |
| Gla | -Carboxy-glutamic acid |
| Glp | Pyroglutamic acid |
| Aep | 3-(2-aminoethoxy)propanoic acid |
| Aea | (2-aminoethoxy)acetic acid |
| IsoGlu-octanoic acid | octanoyl-γ-Glu |
| K-octanoic acid | octanoyl-γ-Lys |
| Dapa(Palm) | Hexadecanoyl-β-Diaminopropionic acid |
| IsoGlu-Palm | hexadecanoyl-γ-Glu |
| C-StBu | S-tert-butylthio-cysteine |
| C-tBu | S-tert-butyl-cysteine |
| N-MeCys, (Me)Cys or αMeCys | N-methyl-cysteine |
| a-MeCys, aMeCys, or α-MeCys | α-methyl-cysteine |
| hCys | homo-cysteine |
| Dapa(AcBr) | NY-(bromoacetyl)-2,3- diaminopropionic acid |
| Tle | tert-Leucine |
| Phg | phenylglycine |
| Oic | octahydroindole-2-carboxylic acid |
| Chg | α-cyclohexylglycine |
| GP-(Hyp) | Gly-Pro-Hydroxy Pro |
| Inp | 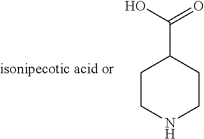 |
| Amc | 4-(aminomethyl)cyclohexane carboxylic acid |
| Betaine | (CH₃)₃NCH₂CH2CO2H |
| D-Npc or D-NPC | (D)-nipecotic acid |
| Npc or NPC | Nipecotic acid |
| (D)Lys, D-Lys, k, or dK | D-Lysine |
| Orn | Ornithine |
| Homoserine or hSer | homoserine |
| Nleu or Nle | Norleucine |
| bhPro | b-homoproline |
| 1-MeHis, His_1Me, His(1-Me), or MeHis | 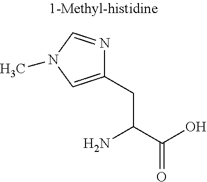 |
| DiIso AmylAmine_CH2 Acid | 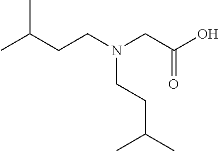 |
| (Me)Glu or Glu_Me | N-Me-glutamic acid |
| 3Pal or 3-Pal | 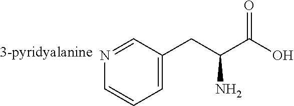 [SMILES: N[C@@H](Cc1cccnc1)C(=O)O] |
| 3Quin or 3-Quin | 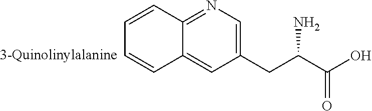 [SMILES: N[C@@H](Cc1cnc2ccccc2c1)C(=O)O] |
| aMeF or a-MePhe or (a-Me)Phe | 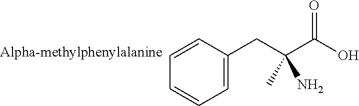 |
| Me_Thr | N-Me-threonine |
| Hyp | 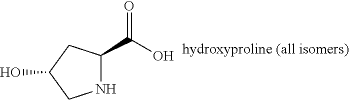 |
| Tet1 | 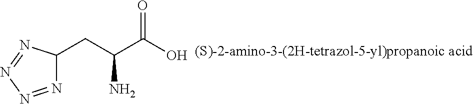 [SMILES: N[C@@H](CC1N=NN=N1)C(=O)O] |
| Tet2 | (S)-2-amino-4-(1H-tetrazol-5-yl)butanoic acid |
| Z_minus | Lys(isoglu_Palm) |
| (Me)Ile or (N-Me)Ile | N-methyl-isoleucine |
| C18_Diacid isoGlu_1Peg2_1Peg2 or C18 Diacid isoGlu 1PEG2 1PEG2 | 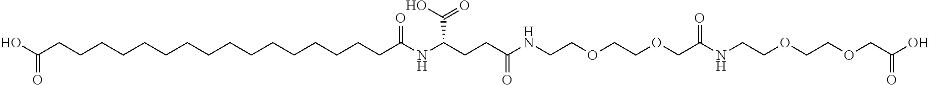 [SMILES: O=C(O)CCCCCCCCCCCCCCCCC(=O)N[C@@H](CCC(=O)NCCOCCOCC(=O)NCCOCCOCC(=O)O)C(=O)O] |
| C18_Diacid Ahx_1Peg2_1Peg2 or C18_Diacid_Ahx_1PEG2 1PEG2 | 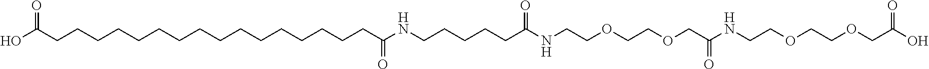 [SMILES: O=C(O)CCCCCCCCCCCCCCCCC(=O)NCCCCCC(=O)NCCOCCOCC(=O)NCCOCCOCC(=O)O] |
| PropanoicP, ProtanoicPro or Ppa | 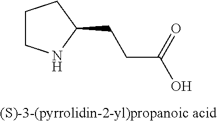 |
| ButanoicP, ButanoicPro, or Pba | 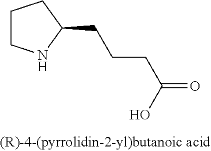 [SMILES: O=C(O)CCC[C@@H]1CCCN1] |
| Gaba or GABA | γ-aminobutyric acid (NH2CH2CH2CH2CO2H) |
| alkanoyl | —C(O)-alkanyl |
| alkenoyl | —C(O)-alkenyl |
| iso Asp | 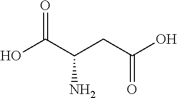 |
| Lys(Gal) or Lys_Gal | 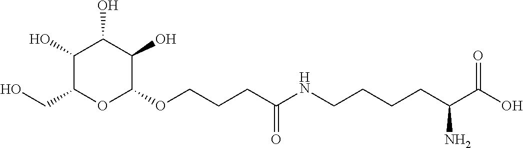 [SMILES: N[C@@H](CCCCNC(=O)CCCO[C@@H]1O[C@H](CO)[C@H](O)[C@H](O)[C@H]1O)C(=O)O] |
| dLys_Gal | 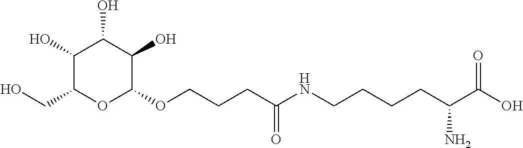 [SMILES: N[C@H](CCCCNC(=O)CCCO[C@@H]1O[C@H](CO)[C@H](O)[C@H](O)[C@H]1O)C(=O)O] |
| Lys_1PEG2_1PEG2_Dap_C18_Diacid | 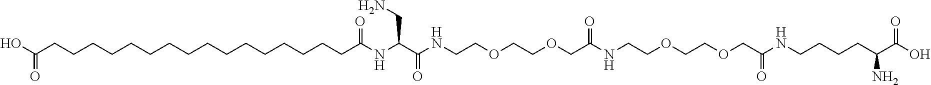 [SMILES: NC[C@H](NC(=O)CCCCCCCCCCCCCCCCC(=O)O)C(=O)NCCOCCOCC(=O)NCCOCCOCC(=O)NCCCC[C@H](N)C(=O)O] |
| Lys_Acrylamide | 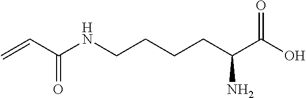 |
| dK_Acrylamide | 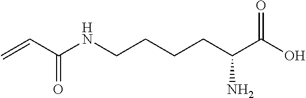 |
| Lys_PEG11_OMe | 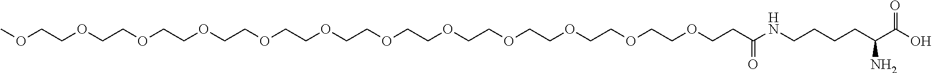 [SMILES: COCCOCCOCCOCCOCCOCCOCCOCCOCCOCCOCCOCCC(=O)NCCCC[C@H](N)C(=O)O] |
| dLys_PEG11_OMe | 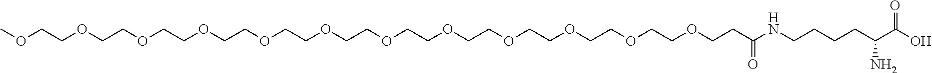 [SMILES: COCCOCCOCCOCCOCCOCCOCCOCCOCCOCCOCCOCCC(=O)NCCCC[C@@H](N)C(=O)O] |
| dLys_PEG8′_OMe or dLYs_PEG7_OMe) | 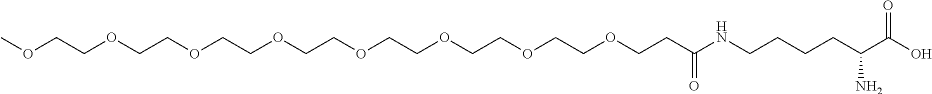 [SMILES: COCCOCCOCCOCCOCCOCCOCCOCCC(=O)NCCCC[C@@H](N)C(=O)O] |
| dLys_PEG4′_OMe or dLYs_PEG3_OMe | 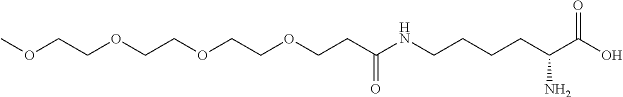 [SMILES: COCCOCCOCCOCCC(=O)NCCCC[C@@H](N)C(=O)O] |
| PEG20K | 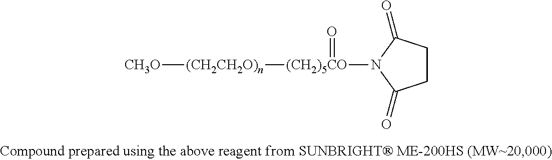 |
| | 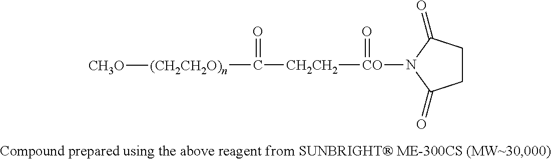 |
| PEG40KB | 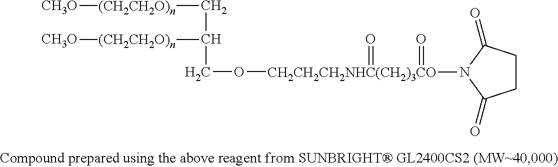 |

TABLE 1B
Abbreviations of Non-Natural Amino Acids and Chemical Moieties

| Abbreviation | Definition |
|---|---|
| (D)Lys(Ac) | N6-acetyl-D-lysine |
| 2Nal | (S)-2-amino-3-(naphthalen-2-yl)propanoic acid |
| aMePhe | alpha-methyl-L-phenylalanine |
| bhLys | (S)-3,7-diaminoheptanoic acid |
| Cys(Ckam) | S-(((2S)-2-amino-3-((1,6-diamino-1-oxohexan-2-yl)amino)-3- |
| | oxopropyl)thio)-L-cysteine |
| Cys(SKGCKam) | S-(((2S)-2-(2-((S)-6-amino-2-((S)-2-amino-3- |
| | hydroxypropanamido)hexanamido)acetamido)-3-((1,6-diamino- |
| | 1-oxohexan-2-yl)amino)-3-oxopropyl)thio)-L-cysteine |
| Hcy | L-homocysteine |
| Lys(AFDye647) | 1-(6-(((S)-5-amino-5-carboxypentyl)amino)-6-oxohexyl)-2- |
| | ((1E,3E)-5-((E)-3,3-dimethyl-5-sulfo-1-(3-sulfopropyl)indolin- |
| | 2-ylidene)penta-1,3-dien-1-yl)-3-methyl-3-(4-sulfobutyl)-3H- |
| | indol-1-ium-5-sulfonate |
| Lys(IsoGlu_10OHPalm) | N6-((4R)-4-carboxy-4-(10-hydroxyhexadecanamido)butanoyl)- |
| | L-lysine |
| Lys(IsoGlu_11OHPalm) | N6-((4R)-4-carboxy-4-(12-hydroxyhexadecanamido)butanoyl)- |
| | L-lysine |
| Lys(IsoGlu_12OHPalm) | N6-((4R)-4-carboxy-4-(14-hydroxyhexadecanamido)butanoyl)- |
| | L-lysine |
| Lys(IsoGlu_13OHPalm) | N6-((4R)-4-carboxy-4-(13-hydroxyhexadecanamido)butanoyl)- |
| | L-lysine |
| Lys(IsoGlu_14OHPalm) | (2R)-5-(((S)-5-amino-5-carboxypentyl)amino)-2-(14- |
| | hydroxyhexadecanamido)hex-5-enoic acid |
| Lys(IsoGlu_15OHPalm) | N6-((4R)-4-carboxy-4-(15-hydroxyhexadecanamido)butanoyl)- |
| | L-lysine |
| Lys(IsoGlu_16F3Palm) | N6-((R)-4-carboxy-4-(16,16,16- |
| | trifluorohexadecanamido)butanoyl)-L-lysine |
| Lys(IsoGlu_2OHPalm) | N6-((4R)-4-carboxy-4-(2-hydroxyhexadecanamido)butanoyl)- |
| | L-lysine |
| Lys(IsoGlu_4OHPalm) | N6-((4R)-4-carboxy-4-(4-hydroxyhexadecanamido)butanoyl)- |
| | L-lysine |
| Lys(IsoGlu_5OHPalm) | N6-((4R)-4-carboxy-4-(5-hydroxyhexadecanamido)butanoyl)- |
| | L-lysine |
| Lys(IsoGlu_6OHPalm) | N6-((4R)-4-carboxy-4-(6-hydroxyhexadecanamido)butanoyl)- |
| | L-lysine |
| Lys(IsoGlu_7OHPalm) | N6-((4R)-4-carboxy-4-(7-hydroxyhexadecanamido)butanoyl)- |
| | L-lysine |
| Lys(IsoGlu_8OHPalm) | N6-((4R)-4-carboxy-4-(8-hydroxyhexadecanamido)butanoyl)- |
| | L-lysine |
| Lys(IsoGlu_9OHPalm) | N6-((4R)-4-carboxy-4-(9-hydroxyhexadecanamido)butanoyl)- |
| | L-lysine |
| Lys(IsoGlu_Palm)_OH | N6-((S)-4-carboxy-4-(16-hydroxyhexadecanamido)butanoyl)-L- |
| | lysine |
| Phe(13C9_15N) | L-phenylalanine-13C9-15N |
| Phe(3,4-diF) | (S)-2-amino-3-(3,4-difluorophenyl)propanoic acid |
| Phe(4-(2-aminoethoxy) | (S)-2-amino-3-(4-(2-aminoethoxy)phenyl)propanoic acid |
| Phe(4-CN) | (S)-2-amino-3-(4-cyanophenyl)propanoic acid |
| Phe(4-COOH) | (S)-4-(2-amino-2-carboxyethyl)benzoic acid |
| Phe(4-tBu) | (S)-2-amino-3-(4-(tert-butyl)phenyl)propanoic acid |

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). In the case of less common or non-naturally occurring amino acids, unless they are referred to by their full name (e.g., sarcosine, ornithine, etc.), frequently employed three- or four-character codes are employed for residues thereof, including, Sar or Sarc (sarcosine, i.e. N-methylglycine), Aib (α-aminoisobutyric acid), Daba (2,4-diaminobutanoic acid), Dapa (2,3-diaminopropanoic acid), γ-Glu (γ-glutamic acid), pGlu (pyroglutamic acid), Gaba (γ-aminobutanoic acid), β-Pro (pyrrolidine-3-carboxylic acid), 8Ado (8-amino-3,6-dioxaoctanoic acid), Abu (4-aminobutyric acid), bhPro (β-homo-proline), bhPhe (β-homo-L-phenylalanine), bhAsp (β-homo-aspartic acid]), Dpa (β,β diphenylalanine), Ida (Iminodiacetic acid), hCys (homocysteine), bhDpa (β-homo-β,β-diphenylalanine).

Furthermore, R¹ can in all sequences be substituted with isovaleric acid or equivalent. In some embodiments, wherein a peptide of the present invention is conjugated to an acidic compound such as, e.g., isovaleric acid, isobutyric acid, valeric acid, and the like, the presence of such a conjugation is referenced in the acid form. So, for example, but not to be limited in any way, instead of indicating a conjugation of isovaleric acid to a peptide by referencing isovaleroyl, in some embodiments, the present application may reference such a conjugation as isovaleric acid.

It is understood that for each of the hepcidin analogue formulas provided herein, bonds may be indicated by a “-” or implied based on the formula and constituent(s). For example, “B7(L1Z)” is understood to include a bond between B7 and L1 if L1 is present, or between B7 and Z if L1 is absent. Similarly, “B5(L1Z)” is understood to include a bond between B5 and L1 if L1 is present, or between B5 and Z if L1 is absent. In addition, it is understood that a bond exists between L1 and Z when both are present. Accordingly, definitions of certain substituents, such as e.g., B7, L1 and J, may include “-” before and/or after the defined substituent, but in each instance, in it understood that the substituent is bonded to other substituents via a single bond. For example, where “J” is defined as Lys, D-Lys, Arg, Pro, -Pro-Arg-, etc., it is understood that J is bound to Xaa2 and Y1 via single bonds. Thus, definitions of substituents may include or may not include “-”, but are still understood to be bonded to adjacent substituents.

The term “L-amino acid,” as used herein, refers to the “L” isomeric form of a peptide, and conversely the term “D-amino acid” refers to the “D” isomeric form of a peptide. In certain embodiments, the amino acid residues described herein are in the “L” isomeric form, however, residues in the “D” isomeric form can be substituted for any L-amino acid residue, as long as the desired functional is retained by the peptide.

Unless otherwise indicated, reference is made to the L-isomeric forms of the natural and unnatural amino acids in question possessing a chiral center. Where appropriate, the D-isomeric form of an amino acid is indicated in the conventional manner by the prefix “D” before the conventional three-letter code (e.g. Dasp, (D)Asp or D-Asp; Dphe, (D)Phe or D-Phe).

As used herein, a “lower homolog of Lys” refers to an amino acid having the structure of Lysine but with one or more fewer carbons in its side chain as compared to Lysine.

As used herein, a “higher homolog of Lys” refers to an amino acid having the structure of Lysine but with one or more additional carbon atoms in its side chain as compared to Lysine.

The term “DRP,” as used herein, refers to disulfide rich peptides.

The term “dimer,” as used herein, refers broadly to a peptide comprising two or more monomer subunits. Certain dimers comprise two DRPs. Dimers of the present invention include homodimers and heterodimers. A monomer subunit of a dimer may be linked at its C- or N-terminus, or it may be linked via internal amino acid residues. Each monomer subunit of a dimer may be linked through the same site, or each may be linked through a different site (e.g., C-terminus, N-terminus, or internal site).

The term “isostere replacement” or “isostere substitution” are used interchangeably herein to refer to any amino acid or other analog moiety having chemical and/or structural properties similar to a specified amino acid. In certain embodiments, an isostere replacement is a conservative substitution with a natural or unnatural amino acid.

The term “cyclized,” as used herein, refers to a reaction in which one part of a polypeptide molecule becomes linked to another part of the polypeptide molecule to form a closed ring, such as by forming a disulfide bridge or other similar bond.

The term “subunit,” as used herein, refers to one of a pair of polypeptide monomers that are joined to form a dimer peptide composition.

The term “linker moiety,” as used herein, refers broadly to a chemical structure that is capable of linking or joining together two peptide monomer subunits to form a dimer.

The term “solvate” in the context of the present invention refers to a complex of defined stoichiometry formed between a solute (e.g., a hepcidin analogue or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

As used herein, a “disease of iron metabolism” includes diseases where aberrant iron metabolism directly causes the disease, or where iron blood levels are dysregulated causing disease, or where iron dysregulation is a consequence of another disease, or where diseases can be treated by modulating iron levels, and the like. More specifically, a disease of iron metabolism according to this disclosure includes iron overload diseases, iron deficiency disorders, disorders of iron biodistribution, other disorders of iron metabolism and other disorders potentially related to iron metabolism, etc. Diseases of iron metabolism include hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, sickle cell disease, polycythemia vera (primary and secondary), myelodysplasia, pyruvate kinase deficiency, iron-deficiency anemia, iron-refractory iron deficiency anemia, anemia of chronic kidney disease, erythropoietin resistance, iron deficiency of obesity, other anemias, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer, hepatitis, cirrhosis of liver, pica, chronic renal failure, insulin resistance, diabetes, atherosclerosis, neurodegenerative disorders, multiple sclerosis, Parkinson's disease, Huntington's disease, and Alzheimer's disease.

In some embodiments, the disease and disorders are related to iron overload diseases such as iron hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sickle cell disease, polycythemia vera (primary and secondary), mylodysplasia, and pyruvate kinase deficiency,.

In some embodiments, the hepcidin analogues of the invention are used to treat diseases and disorders that are not typically identified as being iron related. For example, hepcidin is highly expressed in the murine pancreas suggesting that diabetes (Type I or Type II), insulin resistance, glucose intolerance and other disorders may be ameliorated by treating underlying iron metabolism disorders. See Ilyin, G. et al. (2003) FEBS Lett. 542 22-26, which is herein incorporated by reference. As such, peptides of the invention may be used to treat these diseases and conditions. Those skilled in the art are readily able to determine whether a given disease can be treated with a peptide according to the present invention using methods known in the art, including the assays of WO 2004092405, which is herein incorporated by reference, and assays which monitor hepcidin, hemojuvelin, or iron levels and expression, which are known in the art such as those described in U.S. Pat. No. 7,534,764, which is herein incorporated by reference.

In certain embodiments of the present invention, the diseases of iron metabolism are iron overload diseases, which include hereditary hemochromatosis, iron-loading anemias, alcoholic liver diseases and chronic hepatitis C.

The term “pharmaceutically acceptable salt,” as used herein, represents salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C1-6-alkyl or optionally substituted C2-6-alkenyl. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

The term “N(alpha)Methylation”, as used herein, describes the methylation of the alpha amine of an amino acid, also generally termed as an N-methylation.

The term “sym methylation” or “Arg-Me-sym”, as used herein, describes the symmetrical methylation of the two nitrogens of the guanidine group of arginine. Further, the term “asym methylation” or “Arg-Me-asym” describes the methylation of a single nitrogen of the guanidine group of arginine.

The term “acylating organic compounds”, as used herein refers to various compounds with carboxylic acid functionality that are used to acylate the N-terminus of an amino acid subunit prior to forming a C-terminal dimer. Non-limiting examples of acylating organic compounds include cyclopropylacetic acid, 4-Fluorobenzoic acid, 4-fluorophenylacetic acid, 3-Phenylpropionic acid, Succinic acid, Glutaric acid, Cyclopentane carboxylic acid, 3,3,3-trifluoropropeonic acid, 3-Fluoromethylbutyric acid, Tetrahedro-2H-Pyran-4-carboxylic acid.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

As used herein, a “therapeutically effective amount” of the peptide agonists of the invention is meant to describe a sufficient amount of the peptide agonist to treat an hepcidin-related disease, including but not limited to any of the diseases and disorders described herein (for example, a disease of iron metabolism). In particular embodiments, the therapeutically effective amount will achieve a desired benefit/risk ratio applicable to any medical treatment.

### Peptide Analogues of Hepcidin

The present invention provides peptide analogues of hepcidin, which may be monomers or dimers (collectively “hepcidin analogues”).

In some embodiments, a hepcidin analogue of the present invention binds ferroportin, e.g., human ferroportin. In certain embodiments, hepcidin analogues of the present invention specifically bind human ferroportin. As used herein, “specifically binds” refers to a specific binding agent's preferential interaction with a given ligand over other agents in a sample. For example, a specific binding agent that specifically binds a given ligand, binds the given ligand, under suitable conditions, in an amount or a degree that is observable over that of any nonspecific interaction with other components in the sample. Suitable conditions are those that allow interaction between a given specific binding agent and a given ligand. These conditions include pH, temperature, concentration, solvent, time of incubation, and the like, and may differ among given specific binding agent and ligand pairs, but may be readily determined by those skilled in the art. In some embodiments, a hepcidin analogue of the present invention binds ferroportin with greater specificity than a hepcidin reference compound (e.g., any one of the hepcidin reference compounds provided herein). In some embodiments, a hepcidin analogue of the present invention exhibits ferroportin specificity that is at least about 10%, 20%, 30%, 40%, 50%, 60%, 70%, 80%, 90%, 100%, 200%, 300%, 400%, 500%, 700%, 1000%, or 10,000% higher than a hepcidin reference compound (e.g., any one of the hepcidin reference compounds provided herein. In some embodiments, a hepcidin analogue of the present invention exhibits ferroportin specificity that is at least about 5 fold, or at least about 10, 20, 50, or 100 fold higher than a hepcidin reference compound (e.g., any one of the hepcidin reference compounds provided herein.

In certain embodiments, a hepcidin analogue of the present invention exhibits a hepcidin activity. In some embodiments, the activity is an in vitro or an in vivo activity, e.g., an in vivo or an in vitro activity described herein. In some embodiments, a hepcidin analogue of the present invention exhibits at least about 50%, 60%, 70%, 80%, 90%, 95%, 97%, 98%, 99%, or greater than 99% of the activity exhibited by a hepcidin reference compound (e.g., any one of the hepcidin reference compounds provided herein.

In some embodiments, a hepcidin analogue of the present invention exhibits at least about 50%, 60%, 70%, 80%, 90%, 95%, 97%, 98%, 99%, or greater than 99% of the ferroportin binding ability that is exhibited by a hepcidin reference compound. In some embodiments, a hepcidin analogue of the present invention has a lower EC₅₀ or IC₅₀ (i.e., higher binding affinity) for binding to ferroportin, (e.g., human ferroportin) compared to a hepcidin reference compound. In some embodiments, a hepcidin analogue the present invention has an EC₅₀ in a ferroportin competitive binding assay which is at least about 10%, 20%, 30%, 40%, 50%, 60%, 70%, 80%, 90%, 100%, 200%, 300%, 400%, 500%, 700%, or 1000% lower than a hepcidin reference compound.

In certain embodiments, a hepcidin analogue of the present invention exhibits increased hepcidin activity as compared to a hepcidin reference compound. In some embodiments, the activity is an in vitro or an in vivo activity, e.g., an in vivo or an in vitro activity described herein. In certain embodiments, the hepcidin analogue of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater hepcidin activity than a hepcidin reference compound. In certain embodiments, the hepcidin analogue of the present invention exhibits at least about 10%, 20%, 30%, 40%, 50%, 60%, 70%, 80%, 90%, 95%, 97%, 98%, 99% or greater than 99%, 100%, 200% 300%, 400%, 500%, 700%, or 1000% greater activity than a hepcidin reference compound.

In some embodiments, a peptide analogue of the present invention exhibits at least about 50%, 60%, 70%, 80%, 90%, 95%, 97%, 98%, 99%, or greater than 99%, 100%, 200% 300%, 400%, 500%, 700%, or 1000% greater in vitro activity for inducing the degradation of human ferroportin protein as that of a hepcidin reference compound, wherein the activity is measured according to a method described herein.

In some embodiments, a peptide or a peptide dimer of the present invention exhibits at least about 50%, 60%, 70%, 80%, 90%, 95%, 97%, 98%, 99%, or greater than 99%, 100%, 200% 300%, 400%, 500%, 700%, or 1000% greater in vivo activity for inducing the reduction of free plasma iron in an individual as does a hepcidin reference compound, wherein the activity is measured according to a method described herein.

In some embodiments, the activity is an in vitro or an in vivo activity, e.g., an in vivo or an in vitro activity described herein. In certain embodiments, a hepcidin analogue of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, 500%, 700%, or 1000% greater activity than a hepcidin reference compound, wherein the activity is an in vitro activity for inducing the degradation of ferroportin, e.g., as measured according to the Examples herein; or wherein the activity is an in vivo activity for reducing free plasma iron, e.g., as measured according to the Examples herein.

In some embodiments, the hepcidin analogues of the present invention mimic the hepcidin activity of Hep25, the bioactive human 25-amino acid form, are herein referred to as “mini-hepcidins”. As used herein, in certain embodiments, a compound (e.g., a hepcidin analogue) having a “hepcidin activity” means that the compound has the ability to lower plasma iron concentrations in subjects (e.g. mice or humans), when administered thereto (e.g. parenterally injected or orally administered), in a dose-dependent and time-dependent manner. See e.g. as demonstrated in Rivera et al. (2005), Blood 106:2196-9. In some embodiments, the peptides of the present invention lower the plasma iron concentration in a subject by at least about 1.2, 1.5, 2, 3, 4, 5, 6, 7, 8, 9, or 10-fold, or at least about 5%, 10%, 20%, 25%, 30%, 40%, 50%, 60%, 70%, 80%, 90%, or about 99%.

In some embodiments, the hepcidin analogues of the present invention have in vitro activity as assayed by the ability to cause the internalization and degradation of ferroportin in a ferroportin-expressing cell line as taught in Nemeth et al. (2006) Blood 107:328-33. In some embodiments, in vitro activity is measured by the dose-dependent loss of fluorescence of cells engineered to display ferroportin fused to green fluorescent protein as in Nemeth et al. (2006) Blood 107:328-33. Aliquots of cells are incubated for 24 hours with graded concentrations of a reference preparation of Hep25 or a mini-hepcidin. As provided herein, the EC₅₀ values are provided as the concentration of a given compound (e.g. a hepcidin analogue peptide or peptide dimer of the present invention) that elicits 50% of the maximal loss of fluorescence generated by a reference compound. The EC₅₀ of the Hep25 preparations in this assay range from 5 to 15 nM and in certain embodiments, preferred hepcidin analogues of the present invention have EC₅₀ values in in vitro activity assays of about 1,000 nM or less. In certain embodiments, a hepcidin analogue of the present invention has an EC₅₀ or IC₅₀ in an in vitro activity assay (e.g., as described in Nemeth et al. (2006) Blood 107:328-33 or the Example herein) of less than about any one of 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, a hepcidin analogue or biotherapeutic composition (e.g., any one of the pharmaceutical compositions described herein) has an EC₅₀ or IC₅₀ value of about 1 nM or less.

Other methods known in the art for calculating the hepcidin activity and in vitro activity of the hepcidin analogues according to the present invention may be used. For example, in certain embodiments, the in vitro activity of the hepcidin analogues or the reference peptides is measured by their ability to internalize cellular ferroportin, which is determined by immunohistochemistry or flow cytometry using antibodies which recognizes extracellular epitopes of ferroportin. Alternatively, in certain embodiments, the in vitro activity of the hepcidin analogues or the reference peptides is measured by their dose-dependent ability to inhibit the efflux of iron from ferroportin-expressing cells that are preloaded with radioisotopes or stable isotopes of iron, as in Nemeth et al. (2006) Blood 107:328-33.

In some embodiments, the hepcidin analogues of the present invention exhibit increased stability (e.g., as measured by half-life, rate of protein degradation) as compared to a hepcidin reference compound. In certain embodiments, the stability of a hepcidin analogue of the present invention is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a hepcidin reference compound. In some embodiments, the stability is a stability that is described herein. In some embodiments, the stability is a plasma stability, e.g., as optionally measured according to the method described herein. In some embodiments, the stability is stability when delivered orally.

In particular embodiments, a hepcidin analogue of the present invention exhibits a longer half-life than a hepcidin reference compound. In particular embodiments, a hepcidin analogue of the present invention has a half-life under a given set of conditions (e.g., temperature, pH) of at least about 5 minutes, at least about 10 minutes, at least about 20 minutes, at least about 30 minutes, at least about 45 minutes, at least about 1 hour, at least about 2 hour, at least about 3 hours, at least about 4 hours, at least about 5 hours, at least about 6 hours, at least about 12 hours, at least about 18 hours, at least about 1 day, at least about 2 days, at least about 4 days, at least about 7 days, at least about 10 days, at least about two weeks, at least about three weeks, at least about 1 month, at least about 2 months, at least about 3 months, or more, or any intervening half-life or range in between, about 5 minutes, about 10 minutes, about 20 minutes, about 30 minutes, about 45 minutes, about 1 hour, about 2 hour, about 3 hours, about 4 hours, about 5 hours, about 6 hours, about 12 hours, about 18 hours, about 1 day, about 2 days, about 4 days, about 7 days, about 10 days, about two weeks, about three weeks, about 1 month, about 2 months, about 3 months, or more, or any intervening half-life or range in between. In some embodiments, the half-life of a hepcidin analogue of the present invention is extended due to its conjugation to one or more lipophilic substituent or half-life extension moiety, e.g., any of the lipophilic substituents or half-life extension moieties disclosed herein. In some embodiments, the half-life of a hepcidin analogue of the present invention is extended due to its conjugation to one or more polymeric moieties, e.g., any of the polymeric moieties or half-life extension moieties disclosed herein. In certain embodiments, a hepcidin analogue of the present invention has a half-life as described above under the given set of conditions wherein the temperature is about 25° C., about 4° C., or about 37° C., and the pH is a physiological pH, or a pH about 7.4.

In certain embodiments, a hepcidin analogue of the present invention, comprising a conjugated half-life extension moiety, has an increased serum half-life following oral, intravenous or subcutaneous administration as compared to the same analogue but lacking the conjugated half-life extension moiety. In particular embodiments, the serum half-life of a hepcidin analogue of the present invention following any of oral, intravenous or subcutaneous administration is at least 12 hours, at least 24 hours, at least 30 hours, at least 36 hours, at least 48 hours, at least 72 hours or at least 168 h. In particular embodiments, it is between 12 and 168 hours, between 24 and 168 hours, between 36 and 168 hours, or between 48 and 168 hours.

In certain embodiments, a hepcidin analogue of the present invention, e.g., a hepcidin analogue comprising a conjugated half-life extension moiety, results in decreased concentration of serum iron following oral, intravenous or subcutaneous administration to a subject. In particular embodiments, the subject's serum iron concentration is decreased to less than 10%, less than 20%, less than 25%, less than 30%, less than 40%, less than 50%, less than 60%, less than 70%, less than 80%, or less than 90% of the serum iron concentration in the absence of administration of the hepcidin analogue to the subject. In particular embodiments, the decreased serum iron concentration remains for a least 1 hour, at least 4 hours, at least 10 hours, at least 12 hours, at least 24 hours, at least 36 hours, at least 48 hours, or at least 72 hours following administration to the subject. In particular embodiments, it remains for between 12 and 168 hours, between 24 and 168 hours, between 36 and 168 hours, or between 48 and 168 hours. In one embodiment, the serum iron concentration of the subject is reduced to less than 20% at about 4 hours or about 10 hours following administration to the subject, e.g., intravenously, orally, or subcutaneously. In one embodiment, the serum iron concentration of the subject is reduced to less than 50% or less than 60% for about 24 to about 30 hours following administration, e.g., intravenously, orally, or subcutaneously.

In some embodiments, the half-life is measured in vitro using any suitable method known in the art, e.g., in some embodiments, the stability of a hepcidin analogue of the present invention is determined by incubating the hepcidin analogue with pre-warmed human serum (Sigma) at 37° C. Samples are taken at various time points, typically up to 24 hours, and the stability of the sample is analyzed by separating the hepcidin analogue from the serum proteins and then analyzing for the presence of the hepcidin analogue of interest using LC-MS.

In some embodiments, the stability of the hepcidin analogue is measured in vivo using any suitable method known in the art, e.g., in some embodiments, the stability of a hepcidin analogue is determined in vivo by administering the peptide or peptide dimer to a subject such as a human or any mammal (e.g., mouse) and then samples are taken from the subject via blood draw at various time points, typically up to 24 hours. Samples are then analyzed as described above in regard to the in vitro method of measuring half-life. In some embodiments, in vivo stability of a hepcidin analogue of the present invention is determined via the method disclosed in the Examples herein.

In some embodiments, the present invention provides a hepcidin analogue as described herein, wherein the hepcidin analogue exhibits improved solubility or improved aggregation characteristics as compared to a hepcidin reference compound. Solubility may be determined via any suitable method known in the art. In some embodiments, suitable methods known in the art for determining solubility include incubating peptides (e.g., a hepcidin analogue of the present invention) in various buffers (Acetate pH4.0, Acetate pH5.0, Phos/Citrate pH5.0, Phos Citrate pH6.0, Phos pH 6.0, Phos pH 7.0, Phos pH7.5, Strong PBS pH 7.5, Tris pH7.5, Tris pH 8.0, Glycine pH 9.0, Water, Acetic acid (pH 5.0 and other known in the art) and testing for aggregation or solubility using standard techniques. These include, but are not limited to, visual precipitation, dynamic light scattering, Circular Dichroism and fluorescent dyes to measure surface hydrophobicity, and detect aggregation or fibrillation, for example. In some embodiments, improved solubility means the peptide (e.g., the hepcidin analogue of the present invention) is more soluble in a given liquid than is a hepcidin reference compound.

In certain embodiments, the present invention provides a hepcidin analogue as described herein, wherein the hepcidin analogue exhibits a solubility that is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a hepcidin reference compound in a particular solution or buffer, e.g., in water or in a buffer known in the art or disclosed herein.

In certain embodiments, the present invention provides a hepcidin analogue as described herein, wherein the hepcidin analogue exhibits decreased aggregation, wherein the aggregation of the peptide in a solution is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold less or at least about 10%, 20%, 30%, 40%, 50%, 60%, 70%, 80%, 90%, 100%, 200%, 300%, 400%, or 500% less than a hepcidin reference compound in a particular solution or buffer, e.g., in water or in a buffer known in the art or disclosed herein.

In some embodiments, the present invention provides a hepcidin analogue, as described herein, wherein the hepcidin analogue exhibits less degradation (i.e., more degradation stability), e.g., greater than or about 10% less, greater than or about 20% less, greater than or about 30% less, greater than or about 40 less, or greater than or about 50% less than a hepcidin reference compound. In some embodiments, degradation stability is determined via any suitable method known in the art. In some embodiments, suitable methods known in the art for determining degradation stability include the method described in Hawe et al J Pharm Sci, VOL. 101, NO. 3, 2012, p 895-913, incorporated herein in its entirety. Such methods are in some embodiments used to select potent sequences with enhanced shelf lives.

In some embodiments, the hepcidin analogue of the present invention is synthetically manufactured. In other embodiments, the hepcidin analogue of the present invention is recombinantly manufactured.

The various hepcidin analogue monomer and dimer peptides of the invention may be constructed solely of natural amino acids. Alternatively, these hepcidin analogues may include unnatural or non-natural amino acids including, but not limited to, modified amino acids. In certain embodiments, modified amino acids include natural amino acids that have been chemically modified to include a group, groups, or chemical moiety not naturally present on the amino acid. The hepcidin analogues of the invention may additionally include D-amino acids. Still further, the hepcidin analogue peptide monomers and dimers of the invention may include amino acid analogs. In particular embodiments, a peptide analogue of the present invention comprises any of those described herein, wherein one or more natural amino acid residues of the peptide analogue is substituted with an unnatural or non-natural amino acid, or a D-amino acid.

In certain embodiments, the hepcidin analogues of the present invention include one or more modified or unnatural amino acids. For example, in certain embodiments, a hepcidin analogue includes one or more of Daba, Dapa, Pen, Sar, Cit, Cav, HLeu, 2-Nal, 1-Nal, d-1-Nal, d-2-Nal, Bip, Phe(4-OMe), Tyr(4-OMe), βhTrp, βhPhe, Phe(4-CF₃), 2-2-Indane, 1-1-Indane, Cyclobutyl, βhPhe, hLeu, Gla, Phe(4-NH₂), hPhe, 1-Nal, Nle, 3-3-diPhe, cyclobutyl-Ala, Cha, Bip, j-Glu, Phe(4-Guan), homo amino acids, D-amino acids, and various N-methylated amino acids. One having skill in the art will appreciate that other modified or unnatural amino acids, and various other substitutions of natural amino acids with modified or unnatural amino acids, may be made to achieve similar desired results, and that such substitutions are within the teaching and spirit of the present invention.

The present invention includes any of the hepcidin analogues described herein, e.g., in a free or a salt form.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹3C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements. In particular embodiments, the compounds are isotopically substituted with deuterium. In more particular embodiments, the most labile hydrogens are substituted with deuterium.

The hepcidin analogues of the present invention include any of the peptide monomers or dimers described herein linked to a linker moiety, including any of the specific linker moieties described herein.

The hepcidin analogues of the present invention include peptides, e.g., monomers or dimers, comprising a peptide monomer subunit having at least 85%, at least 90%, at least 92%, at least 94%, at least 95%, at least 98%, or at least 99% amino acid sequence identity to a hepcidin analogue peptide sequence described herein (e.g., any one of the peptides disclosed herein), including but not limited to any of the amino acid sequences shown in Tables 2 and 3.

In certain embodiments, a peptide analogue of the present invention, or a monomer subunit of a dimer peptide analogue of the present invention, comprises or consists of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 18 amino acid residues, 8 to 18 amino acid residues, 9 to 18 amino acid residues, or 10 to 18 amino acid residues, and, optionally, one or more additional non-amino acid moieties, such as a conjugated chemical moiety, e.g., a half-life extension moiety, a PEG or linker moiety. In particular embodiments, a monomer subunit of a hepcidin analogue comprises or consists of 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid residues. In particular embodiments, a monomer subunit of a hepcidin analogue of the present invention comprises or consists of 10 to 18 amino acid residues and, optionally, one or more additional non-amino acid moieties, such as a conjugated chemical moiety, e.g., a PEG or linker moiety. In various embodiments, the monomer subunit comprises or consists of 7 to 35 amino acid residues, 9 to 18 amino acid residues, or 10 to 18 amino acid residues. In particular embodiments of any of the various Formulas described herein, X comprises or consists of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 18 amino acid residues, 8 to 18 amino acid residues, 9 to 18 amino acid residues, or 10 to 18 amino acid residues.

In particular embodiments, a hepcidin analogue or dimer of the present invention does not include any of the compounds described in PCT/US2014/030352 or PCT/US2015/038370.

### Peptide Hepcidin Analogues

In certain embodiments, hepcidin analogues of the present invention comprise a single peptide subunit, optionally conjugated to a half-life extension moiety. In certain embodiments, these hepcidin analogues form cyclized structures through intramolecular disulfide or other bonds.

In one aspect, the present invention includes a hepcidin analogue comprising a peptide of Formula (W-I):

R¹-X1-Thr-His-X4-B2-X6-X7-Xaa1-X9-Xaa2-J-Y1-Y2-R² (W-I)
or a peptide dimer comprising two peptides according to Formula W-I;
or a pharmaceutically acceptable salt, or a solvate thereof, wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is —NH₂ or —OH;
X1 is Asp, (D)Asp, or substituted Asp;
X4 is F, NMePhe, or Dpa;
Xaa1 is B5; and
   i) B5 is absent, Lys, D-Lys, Spiro_pip, Arg(nitro), Arg(dimethyl), Cit, Pro(4-amino), or Cav; and Xaa2 is B7(L1Z); and B7 is Lys, or D-Lys;
or
   ii) Xaa1 is B5(L1Z); B5 is Lys, D-Lys, NMeLys, Dap or Dap-Dap; and Xaa2 is B7; and B7 is Glu, NMeGlu, or absent;
B2 is Pro, hydroxyPro, hydroxy(D)Pro, or NPC;
X6 is Cys, NMeCys, SMeCys, or aMeCys;
X7 is Ile, Leu, or NMeLeu;
X9 is Phe, bhPhe, or NMePhe;
L1 is absent, Dap, D-Dap, or isoGlu, PEG, Ahx, isoGlu-PEG, PEG-isoGlu, PEG-Ahx, isoGlu-Ahx, isoGlu-PEG-Ahx, or PEG-pep;
Ahx is an aminohexanoic acid moiety; pep is a peptide consisting of one or more amino acids; PEG is —[C(O)—CH₂-(Peg)ₙ-N(H)]ₘ—, or —[C(O)—CH₂—CH₂-(Peg)ₙ-N(H)]ₘ—; and Peg is —OCH₂CH₂—, m is 1, 2, or 3; and n is an integer between 1-100K;
Z is a half-life extension moiety;
J is absent, any amino acid, or a peptide chain consisting of 1-5 amino acids, wherein each amino acid is independently selected from Pro, (D)Pro, hydroxyPro, hydroxy(D)Pro, Arg, MeArg, Lys, (D)Lys, Lys(Ac), (D)Lys(Ac), Ser, MeSer, Sar, and Gly;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
hydroxyPro is Proline substituted with hydroxy, hydroxy(D)Pro is (D)Proline substituted with hydroxy,
wherein
   i) the peptide of formula W-I is optionally PEGylated on one or more R¹, B5, B6, B7, J, Y2, or R²; and
   ii) the peptide is optionally cyclized via a disulfide bond between X6 and Y1;
or the hepcidin analogue is any one of peptides listed in Table 3A or Table 3B, and wherein the peptide is optionally cyclized via a disulfide bond between X6 and Y1.

In one embodiment, X1 is Asp.

In one embodiment, X4 is Dpa.

In one embodiment, Xaa1 is B5(L1Z); B5 is Lys, D-Lys, Dap or Dap-Dap; and Xaa2 is B7; and B7 is Glu, or absent.

In one embodiment, Pro, or NPC.

In one embodiment, X7 is Ile.

In one embodiment, X9 is Phe, or bhPhe.

In one embodiment, J is absent, any amino acid, or a peptide chain consisting of 1-5 amino acids, wherein each amino acid is independently selected from Pro, (D)Pro, hydroxyPro, hydroxy(D)Pro, Arg, MeArg, Lys, (D)Lys, Lys(Ac), (D)Lys(Ac), Ser, MeSer, Sar, and Gly.

In one embodiment, J is Arg, Lys, D-Lys, Spiro_pip, Arg(nitro), Arg(dimethyl), Cit, Pro(4-amino), Cav, Pro-, Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly-(SEQ ID NO:251), -Pro-Lys(Ac)-, -Pro-(D)Lys(Ac)-, -Pro-Arg-Ser-Lys(Ac)-(SEQ ID NO:249), -Pro-Arg-Ser-Lys(Ac)-Sar-(SEQ ID NO:250), -Pro-Arg-Ser-Lys(Ac)-Gly-, -HydroxyPro-Arg-Ser-Lys-Gly-(SEQ ID NO:251), -Pro-MeArg-Ser-Lys-Gly-, -Pro-Arg-MeSer-Lys-Gly-(SEQ ID NO:251), (SEQ ID NO:251), -Pro-Lys(Ac)-Ser-Lys(Ac)-, -Pro-Lys(Ac)-Ser-Lys(Ac)-Gly-, -Pro-Lys(Ac)-Ser-Lys(Ac)-Gly-, -Pro-Lys(Ac)-Ser-Lys(Ac)-Sar-, -Pro-Arg-Ser-MeLys-Gly-, or absent; or J is any amino acid.

In one embodiment, J is Arg, Lys, D-Lys, Spiro_pip, Arg(nitro), Arg(dimethyl), Cit, Pro(4-amino), Cav, Pro-, Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly-(SEQ ID NO:251), or absent; or J is any amino acid.

In one aspect, the present invention includes a hepcidin analogue comprising a peptide of Formula (I):

R¹-Asp-Thr-His-Dpa-B2-Cys-Ile-Xaa1-bhF-Xaa2-J-Y1-Y2-R² (I)
or a peptide dimer comprising two peptides according to Formula I, or a pharmaceutically acceptable salt, or a solvate thereof,
wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is —NH₂ or —OH;
Xaa1 is B5; and
   i) B5 is absent, Lys, D-Lys, Spiro_pip, Arg(nitro), Arg(dimethyl), Cit, Pro(4-amino), or Cav; and Xaa2 is B7(L1Z); and B7 is Lys, or D-Lys;
or
   ii) Xaa1 is B5(L1Z); B5 is Lys, D-Lys, Dap or Dap-Dap; and Xaa2 is B7; and B7 is Glu, D-Lys, or absent;
B2 is Pro, or NPC;
B7 is Lys, or D-Lys;
L1 is absent, Dap, D-Dap, or isoGlu, PEG, Ahx, isoGlu-PEG, PEG-isoGlu, PEG-Ahx, isoGlu-Ahx, isoGlu-PEG-Ahx, or PEG-pep;
Ahx is an aminohexanoic acid moiety; pep is a peptide consisting of one or more amino acids; PEG is —[C(O)—CH₂—(Peg)ₙ-N(H)]ₘ—, or —[C(O)—CH₂—CH₂—(Peg)ₙ-N(H)]ₘ—; and Peg is —OCH₂CH₂—, m is 1, 2, or 3; and n is an integer between 1-100K;
Z is a half-life extension moiety;
J is Arg, Lys, D-Lys, Spiro_pip, Arg(nitro), Arg(dimethyl), Cit, Pro(4-amino), Cav, Pro, -Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly-(SEQ ID NO:251), or absent; or J is any amino acid;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
wherein
   i) the peptide of formula I is optionally PEGylated on one or more R¹, B5, B6, B7, J, Y2, or R²; and
   ii) the peptide is optionally cyclized via a disulfide bond between Cys and Y1.

In one embodiment, the half-life extension moiety is C₁₀-C₂₁ alkanoyl.

In one embodiment, B2 is Pro.

In one embodiment, Xaa1 is B5; B5 is absent, Lys, or D-Lys; and Xaa2 is B7(L1Z); and B7 is Lys, or D-Lys.

In one embodiment, Xaa1 is B5; B5 is absent, Lys, or D-Lys; and Xaa2 is B7(L1Z); and B7 is Lys, or D-Lys; and L1 is attached to N^{ε} of Lys, or D-Lys.

In one embodiment, Xaa1 is B5(L1Z); B5 is Lys, D-Lys, Dap or Dap-Dap; and Xaa2 is B7; and B7 is Glu or absent

In another embodiment, Xaa1 is B5(L1Z); B5 is Lys, D-Lys, Dap or Dap-Dap; and Xaa2 is B7; and B7 is Glu or absent; and L1 is attached to N^{ε} of Lys or N^{β} of Dap.

In one embodiment, the present invention includes a hepcidin analogue comprising a peptide of Formula (IIa):

R¹-Asp-Thr-His-Dpa-Pro-Cys-Ile-B5-bhF-B7(L1Z)-J-Y1-Y2-R² (IIa)

or a peptide dimer comprising two peptides according to Formula IIa, or a pharmaceutically acceptable salt, or a solvate thereof,
wherein R1, B5, B7, L1, Z, J, Y1, Y2 and R2 are as described for Formula (I).

In one embodiment, the present invention includes a hepcidin analogue comprising a peptide of Formula (IIb):

R¹-Asp-Thr-His-Dpa-Pro-Cys-Ile-B5(L1Z)-bhF-B7-J-Y1-Y2-R² (IIb)

or a peptide dimer comprising two peptides according to Formula IIa, or a pharmaceutically acceptable salt, or a solvate thereof,
wherein R1, B5, B7, L1, Z, J, Y1, Y2 and R2 are as described for Formula (I).

In one embodiment, B7(L1Z) is —N(H)C[CH₂(CH₂CH₂CH₂)ₘN(H)L1Z](H)—C(O)—; and wherein m is 0 or 1.

In one embodiment, B7(L1Z) is —N(H)C[CH₂N(H)L1Z](H)—C(O)—.

In one embodiment, B7(L1Z) is —N(H)C[CH₂CH₂CH₂CH₂N(H)L1Z](H)—C(O)—.

In one embodiment, B5(L1Z) is —N(H)C[CH₂(CH₂CH₂CH₂)ₘN(H)L1Z](H)—C(O)—; and wherein m is 0 or 1.

In one embodiment, B5(L1Z) is —N(H)C[CH₂N(H)L1Z](H)—C(O)—.

In one embodiment, B5(L1Z) is —N(H)C[CH₂CH₂CH₂CH₂N(H)L1Z](H)—C(O)—.

In one embodiment, B5 is (D)Lys.

In one embodiment, the present invention includes a hepcidin analogue comprising a peptide of Formula IIIa or IIIb:

R¹-Asp-Thr-His-Dpa-Pro-Cys-Ile-(D)Lys-bhF—N(H)C[CH₂N(H)L1Z](H)—C(O)-J-Y1-Y2-R² (IIIa), or

R¹-Asp-Thr-His-Dpa-Pro-Cys-Ile-(D)Lys-bhF—N(H)C[CH₂CH₂CH₂CH₂N(H)L1Z](H)—C(O)-J-Y1-Y2-R² (IIIb)
or a peptide dimer thereof, or a pharmaceutically acceptable salt thereof;
wherein R¹, R², L1, Z, J, Y1, and Y2 are as in Formula (I).

In one embodiment, R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl. In one embodiment, R² is —NH₂ or —OH;

In a particular embodiment, with respective to the peptide of invention, the peptide is optionally cyclized via a disulfide bond between two Cys.

In one embodiment, with respective to the peptide of invention, —N(H)C[CH₂N(H)L1Z](H)—C(O)— is an L-amino acid. In another embodiment, with respective to the peptide of invention, —N(H)C[CH₂N(H)L1Z](H)—C(O)— is an D-amino acid.

In one embodiment, with respective to the peptide of invention, —N(H)C[CH₂CH₂CH₂CH₂N(H)L1Z](H)—C(O)— is an L-amino acid. In another embodiment, with respective to the peptide of invention, —N(H)C[CH₂CH₂CH₂CH₂N(H)L1Z](H)—C(O)— is an D-amino acid.

In one embodiment, -J-Y1-Y2- is -Cys-, -Pro-Cys-, -Lys-Cys-, -(D)Lys-Cys-, -Dap-Cys-, -Cys-(D)Lys-, -Dap-hCys-, -Pro-Arg-Cys-, -Pro-Arg-Ser-Cys-(SEQ ID NO:253), -Pro-Arg-Ser-Lys-Cys-(SEQ ID NO:254), or -Pro-Arg-Ser-Lys-Sar-Cys-(SEQ ID NO:255).

In one embodiment, -J-Y1-Y2- is -Cys-, -Pro-Cys-, -Lys-Cys-, -(D)Lys-Cys-, -Dap-Cys-, -Cys-(D)Lys-, -Dap-hCys-, -Pro-Arg-Cys-, -Pro-Arg-Ser-Cys-(SEQ ID NO:253), or -Pro-Arg-Ser-Lys-Cys-(SEQ ID NO:254).

In one embodiment, -J-Y1-Y2- is -Cys-, -Pro-Cys-, -Pro-Lys-Cys-, -Pro-(D)Lys-Cys-, -Lys-Cys-, -(D)Lys-Cys-, -Dap-Cys-, -Cys-(D)Lys-, -Dap-hCys-, -Pro-Arg-Cys-, or -Pro-Arg-Ser-Cys-(SEQ ID NO:253).

In another embodiment, -J-Y1-Y2- is -(D)Lys-Cys- or -Lys-Cys-.

In another embodiment, -J-Y1-Y2- is -Cys-(D)Lys-.

In another embodiment, -J-Y1-Y2- is -Pro-Arg-Ser-Lys-Cys-(SEQ ID NO:254).

In another embodiment, -J-Y1-Y2- is -Pro-Arg-Ser-Lys-Cys-Lys-(SEQ ID NO:255).

In another embodiment, -J-Y1-Y2- is -Pro-Cys-.

In another embodiment, -J-Y1-Y2- is -Cys-.

In another embodiment, -J-Y1-Y2- is -(D)Lys-Pen-.

In one embodiment, R² is NH₂. In another embodiment, R² is OH.

In one embodiment, L1 is a single bond. In another embodiment, L1 is iso-Glu.

In another embodiment, L1 is Ahx. In another embodiment, L1 is iso-Glu-Ahx. In another embodiment, PEG. In another embodiment, L1 is iso-Glu-PEG. In another embodiment, L1 is PEG-iso-Glu. In another embodiment, L1 is PEG-Ahx.

In another embodiment, L1 is iso-Glu-PEG-Ahx. In another embodiment, PEG is PEG1, PEG2, PEG3, PEG4, PEG53, or PEG11. In another embodiment, Z is Palm.

In another embodiment, L1 is PEG-pep, and pep is an amino acid.

In another embodiment, L1 is PEG-pep, and pep is a peptide consisting of 2-4 amino acids.

In another embodiment, L1 is Ahx; and Z is Palm.

In another embodiment, L1 is PEG11; and Z is Palm.

In another embodiment, L1 is Dap; and Z is Palm.

In another embodiment, L1 is dDap; and Z is Palm.

In one embodiment, PEG is —[C(O)—CH₂—(Peg)ₙ-N(H)]ₘ—, or —[C(O)—CH₂—CH₂-(Peg)ₙ-N(H)]ₘ—; and Peg is —OCH₂CH₂—, m is 1, 2, or 3; and n is an integer between 1-100, or is 10K, 20K, or 30K.

In one embodiment, m is 1. In another embodiment, m is 2.

In one embodiment, n is 2. In another embodiment, n is 4. In another embodiment, n is 8. In another embodiment, n is 11. In another embodiment, n is 12. In another embodiment, n is 20K.

In one embodiment, PEG is 1Peg2; and 1Peg2 is —C(O)—CH₂—(Peg)₂-N(H)—.

In another embodiment, PEG is 2Peg2; and 2Peg2 is —C(O)—CH₂—CH₂—(Peg)₂-N(H)—.

In another embodiment, PEG is 1Peg2-1Peg2; and each 1Peg2 is —C(O)—CH₂—CH₂—(Peg)₂-N(H)—.

In another embodiment, PEG is 1Peg2-1Peg2; and 1Peg2-1Peg2 is —[(C(O)—CH₂—(OCH₂CH₂)₂—NH—C(O)—CH₂—(OCH₂CH₂)₂—NH—]—.

In another embodiment, PEG is 2Peg4; and 2Peg4 is —C(O)—CH₂—CH₂—(Peg)₄-N(H)—, or —[C(O)—CH₂—CH₂—(OCH₂CH₂)₄—NH]—.

In another embodiment, PEG is 1Peg8; and 1Peg8 is —C(O)—CH₂—(Peg)₈-N(H)—, or —[C(O)—CH₂—(OCH₂CH₂)₈—NH]—.

In another embodiment, PEG is 2Peg8; and 2Peg8 is —C(O)—CH₂—CH₂—(Peg)₈-N(H)—, or —[C(O)—CH₂—CH₂—(OCH₂CH₂)₈—NH]—.

In another embodiment, PEG is 1Peg11; and 1Peg11 is —C(O)—CH₂—(Peg)₁₁-N(H)—, or —[C(O)—CH₂—(OCH₂CH₂)₁₁—NH]—.

In another embodiment, PEG is 2Peg11; and 2Peg11 is —C(O)—CH₂—CH₂—(Peg)₁₁-N(H)—, or —[C(O)—CH₂—CH₂—(OCH₂CH₂)₁₁—NH]—.

In another embodiment, PEG is 2Peg11′ or 2Peg12; and 2Peg11′ or 2Peg12 is —C(O)—CH₂—CH₂—(Peg)₁₂-N(H)—, or —[C(O)—CH₂—CH₂—(OCH₂CH₂)₁₂—NH]—.

In one embodiment, when PEG is attached to Lys, the —C(O)— of PEG is attached to N^{ε} of Lys.

In one embodiment, when PEG is attached to isoGlu, the —N(H)— of PEG is attached to —C(O)— of isoGlu.

In one embodiment, when PEG is attached to Ahx, the —N(H)— of PEG is attached to —C(O)— of Ahx.

In one embodiment, when PEG is attached to Palm, the —N(H)— of PEG is attached to —C(O)— of Palm.

In a particular embodiment, the peptide is according to formula (A-I):

R¹-Asp-Thr-His-B1-B2-B3-B4-B5-B6-B7(L1Z)-J-Y1-Y2-R² (A-I)

wherein the peptide comprises any of the combinations of B1, B2, B3, B4, B5, B6, B7(L1Z), J, Y1 and Y2 set forth in a row of Table 2, Table 3A or Table 3B, wherein R¹ is IVA, R² is NH₂, and “abs” indicates “absent”. The accompanying sequence identifiers represent the amino acid sequence of peptides according to formula (A-I) having the amino acids indicated for each row of Table 2, Table 3A or Table 3B and indicating the indicated conjugated half-life extension moiety when present.

In certain embodiment, the present invention includes a polypeptide comprising an amino acid sequence set forth in Table 3A or 3B, or having any amino acid sequence with at least 85%, at least 90%, at least 92%, at least 94%, or at least 95% identity to any of these amino acid sequences.

In certain variations of any of the above embodiments, the peptide is other than any of the following:
Isovaleric acid-D-T-H-[Dpa]-P—C—I—[(D)Lys]-[b-homoPhe]-Lys[2PEG11-Palm]-[(D)Lys]-[Cys]-NH₂;
Isovaleric acid-DTH-[Dpa]-PCI-[(D)Lys]-[b-hPhe]-[Lys(2Peg11′-Palm)]-[(D)Lys]-C—NH₂;
Isovaleric acid-DTH-[Dpa]-PCI-[b-hPhe]-[Lys(2Peg11′-Palm)]-[(D)Lys]-C—NH₂; Isovaleric acid-DTH-[Dpa]-PCI-[(D)Lys]-[b-hPhe]-[Lys(Ahx-Palm)]- [Dap]- [N-Me-Cys]-NH₂;
Isovaleric acid-DTH-[Dpa]-PCI-[(D)Lys]-[b-hPhe]-[Lys(Ac)]-[(D)Lys]-C—NH₂;
Isovaleric acid-DTH-[Dpa]-PCI-[(D)Lys-Peg11*]-[b-hPhe]-[Lys(2Peg11′-Palm)]-[(D)Lys-Peg11*]-C—NH₂;
Isovaleric acid-DTH-[Dpa]-PCI-[(D)Lys]-[b-hPhe]-[Lys-Peg 11]-[(D)Lys]-C—NH₂;
Isovaleric acid-DTH-[Dpa]-PCI-[(D)Lys]-[b-hPhe]-[Lys(2Peg11′-Octane)]-[(D)Lys]-C—NH₂;
Isovaleric acid-DTH-[Dpa]-PCI-[(D)Lys]-[b-hPhe]-[Lys(2Peg11′-Lauryl)]-[(D)Lys]-C—NH₂;
Isovaleric acid-DTH-[Dpa]-PCI-[(D)Lys]-[b-hPhe]-[Lys-2Peg11*]-[(D)Lys]-C—NH₂;
Isovaleric acid-DTH-[Dpa]-PCI-[(D)Lys]-[b-hPhe]-[Lys(IsoGlu-Palm)]-[(D)Lys]-C—NH₂;
Isovaleric acid-DTH-[Dpa]-PCI-[(D)Lys-Ac]-[b-hPhe]-[Lys(2Peg11′-Palm)]-[(D)Lys-Ac]-C—NH₂;
Isovaleric acid-DTH-[Dpa]-PCI-[(D)Lys]-[b-hPhe]-[Lys(Dap-Palm)]-C—NH₂;
Isovaleric acid-DTH-[Dpa]-PCI-[(D)Lys]-[b-hPhe]-[Lys(dDap-Palm)]-C—NH₂;
Isovaleric acid-DTH-[Dpa]-PCI-[(D)Lys]-[b-hPhe]-[Dap(Dap-Palm)]-C—NH₂;
Isovaleric acid-DTH-[Dpa]-PCI-[(D)Lys]-[b-hPhe]-[Dap(dDap-Palm)]-C—NH₂;
Isovaleric acid-DTH-[Dpa]-PCI-[(D)Lys]-[b-hPhe]-[Dap(dDap-Palm)]-C—NH₂;
Isovaleric acid-DTH-[Dpa]-PCI-[(D)Lys]-[b-hPhe]-[Lys(Ahx-Palm)]-[(D)Lys]-C—NH₂;
Isovaleric acid-DTH-[Dpa]-PCI-[(D)Lys-Peg11*]-[b-hPhe]-[Lys(Ahx-Palm)]-[(D)Lys-Peg11*]-C—NH₂;
Isovaleric_Acid-dD-T-H-[Dpa]-P—C-I-(D)Lys-bhPhe-[Lys_Peg11_Palm]-(D)Lys-C—NH₂;
Isovaleric_Acid-D-T-H-[Dpa]-P—C-I-(D)Lys-bhPhe-[Lys_Peg11 Palm]-C-(D)Lys-NH₂;
Isovaleric_Acid-D-T-H-[Dpa]-Npc-C-I-(D)Lys-bhPhe-[Lys Peg11_Palm]-C-(D)Lys-NH₂;
Isovaleric_Acid-D-T-H-[Dpa]-P—C-I-(D)Lys-bhPhe-[Lys_Peg11 Palm]-dLys_Ac—C—NH₂;
Isovaleric acid-DTH-Dpa-PCI-Lys[Peg11-Palm]-bhPhe-(D)Lys-C—NH₂;
Isovaleric acid-DTH-Dpa-PCI-Lys[Ahx-Palm]-bhPhe-C—NH₂; or
Isovaleric acid-DTH-Dpa-PCI-Lys[Ahx-Palm]-bhPhe-(D)Lys-C—NH₂;
   or cyclized versions, salts or solvates thereof.

TABLE 2
R¹-Asp-Thr-His-Dpa-B2-Cys-Ile-Xaa1-bhF-Xaa2-J-Y1-Y2-R² (I)

| SEQ | | | | | | | | |
|---|---|---|---|---|---|---|---|---|
| ID | R¹ | B2 | B5 | B7 | J | Y1 | Y2 | R² |
| 1 | IVA | P | Dap(Dap-Palm) | abs | | C | abs | NH₂ |
| 2 | IVA | P | dK | K(Ahx-Palm) | R | C | abs | NH₂ |
| 3 | IVA | P | dK | K(Ahx-Palm) | dK | meC | abs | NH₂ |
| 4 | IVA | Npc | dK | K(Ahx-Palm) | dK | C | abs | NH₂ |
| 5 | IVA | P | K(Ahx-isoGlu- | abs | dK | C | abs | NH₂ |
| | | | Palm) | | | | | |
| 6 | IVA | P | K(isoGlu-Palm) | abs | dK | C | abs | NH₂ |
| 7 | IVA | P | dK | K(Ahx-Behenic Acid) | dK | C | abs | NH₂ |
| 8 | IVA | P | K(Ahx-Behenic Acid) | dK | | C | abs | NH₂ |
| 9 | IVA | P | K(1Peg2-1Peg2- | dK | abs | C | abs | NH₂ |
| | | | isoGlu-Behenic Acid) | | | | | |
| 10 | IVA | P | K(1Peg2-1Peg2-Ahx- | K | dK | C | abs | NH₂ |
| | | | Palm) | | | | | |
| 11 | IVA | P | K(1Peg2-1Peg2-Ahx- | dK | abs | C | abs | NH₂ |
| | | | Palm) | | | | | |
| 12 | IVA | Npc | dK | K(Ahx-Palm) | abs | C | dK | NH₂ |
| 13 | IVA | Npc | dK | K(isoGlu-Palm) | abs | C | dK | NH₂ |
| 14 | IVA | Npc | dK | K(Ahx-isoGlu- | abs | C | dK | NH₂ |
| | | | | Palm) | | | | |
| 15 | IVA | Npc | dK | K(1Peg2-1Peg2-Ahx- | abs | C | dK | NH₂ |
| | | | | Palm) | | | | |
| 16 | IVA | P | dK | K(Palm) | K | C | abs | NH₂ |
| 17 | IVA | P | dK | K(Ahx-Palm) | K | C | abs | NH₂ |
| 18 | IVA | P | abs | K(Ahx-Palm) | abs | C | abs | NH₂ |
| 19 | IVA | Npc | K(isoGlu-Palm) | abs | abs | C | dK | NH₂ |
| 20 | IVA | P | dK | K(Ahx-Palm) | dK | C | abs | NH₂ |
| 21 | IVA | P | dK | K(Ahx-Palm) | Spiro_Pip | C | abs | NH₂ |
| 22 | IVA | P | dK | K(Ahx-Palm) | Arg(nitro) | C | abs | NH₂ |
| 23 | IVA | P | dK | K(Ahx-Palm) | Arg(dimethyl) | C | abs | NH₂ |
| 24 | IVA | P | dK | K(Ahx-Palm) | Cit | C | abs | NH₂ |
| 25 | IVA | P | dK | K(Ahx-Palm) | Pro(4- | C | abs | NH₂ |
| | | | | | Amino) | | | |
| 26 | IVA | P | dK | K(Ahx-Palm) | Cav | C | abs | NH₂ |
| 27 | IVA | P | Spiro_Pip | K(Ahx-Palm) | dK | C | abs | NH₂ |
| 28 | IVA | P | Arg(nitro) | K(Ahx-Palm) | dK | C | abs | NH₂ |
| 29 | IVA | P | Arg(Dimethyl) | K(Ahx-Palm) | dK | C | abs | NH₂ |
| 30 | IVA | P | Cit | K(Ahx-Palm) | dK | C | abs | NH₂ |
| 31 | IVA | P | Pro(4Amino) | K(Ahx-Palm) | dK | C | abs | NH₂ |
| 32 | IVA | P | Cav | K(Ahx-Palm) | dK | C | abs | NH₂ |
| 33 | IVA | P | K(1Peg2-1Peg2-Ahx- | abs | dK | C | abs | NH₂ |
| | | | Palm) | | | | | |

or a peptide dimer thereof; and wherein the peptide is optionally cyclized via a disulfide bond between Cys and Y1.

In certain embodiments of any of the peptide analogues having any of the various Formulae set forth herein, R¹ is selected from methyl, acetyl, formyl, benzoyl, trifluoroacetyl, isovaleryl, isobutyryl, octanyl, and conjugated amides of lauric acid, hexadecanoic acid, and γ-Glu-hexadecanoic acid.

In certain embodiments, the linker between the peptide and the half-life extension moiety is PEG11, Ahx, or any of the others described herein.

In certain embodiments, the half-life extension moiety is Palm.

In certain variations of any of the above embodiments, the peptide is

In certain embodiment, the present invention includes a polypeptide comprising an amino acid sequence set forth in any of Tables 2, 3A, and 3B (with or without the indicated linker moieties and half-life extension moieties), or having any amino acid sequence with at least 85%, at least 90%, at least 92%, at least 94%, or at least 95% identity to any of these amino acid sequences.

In certain embodiment, the present invention provides a cyclized form of any one of the hepcidin analogues disclosed herein or listed in any of Tables 3A, 3B, and 3C comprising a disulfide bond between the two Cys and/or Pen residues. The conjugated half-life extension moiety and the amino acid residue to which it is conjugated are indicated by parentheses and brackets, respectively. Compound ID numbers are indicated by “Compd ID,” and reference compounds are indicated by “Ref. Compd.”

TABLE 3A
Illustrative Monomer Hepcidin Analogues

| | | FPN | SIF | SGF |
|---|---|---|---|---|
| Compd. ID/ | | EC₅₀ | Half life | Half life |
| SEQ ID No. | Peptide | (nM) | (min) | (min) |
| Ref. Cmpd. 1 | Isovaleric acid-DTHFPCIKF-Lys[PEG11- | 30 | <15 (1%) | <15 (6%) |
| | Palm]-PRSKGCK-NH₂ | | | |
| Ref. Cmpd. 2 | Isovaleric acid-DTHFPCIKF-Lys[PEG11- | 13 | <15 (1%) | 26 |
| | Palm]-PRSK-[SAR]-CK-NH₂ | | | |
| 1 | Isovaleric acid-D-T-H-[Dpa]-P-C-I- | 2.54 | | 570 |
| | [Dap(Dap-Palm)]-bhPhe-C-NH₂ | | | |
| 2 | Isovaleric acid-D-T-H-[Dpa]-P-C-I-(D)Lys- | 11.77 | 78 | 144 |
| | bhPhe-[Lys(Ahx-Palm)]-R-C-NH₂ | | | |
| 3 | Isovaleric acid-D-T-H-[Dpa]-P-C-I-(D)Lys- | | 66 | 660 |
| | bhPhe-[Lys(Ahx-Palm)]-(D)Lys-meCys-NH₂ | | | |
| 4 | Isovaleric acid-D-T-H-[Dpa]-[Npc]-C-I- | 6.35 | | |
| | (D)Lys-bhPhe-[Lys(Ahx-Palm)]-(D)Lys-C- | | | |
| | NH₂ | | | |
| 5 | Isovaleric acid-D-T-H-[Dpa]-P-C-I-Lys[Ahx- | 0.75 | | |
| | isoGlu-Palm]-bhPhe--(D)Lys-C-NH₂ | | | |
| 6 | Isovaleric acid-D-T-H-[Dpa]-P-C-I- | 6.16 | | |
| | Lys[isoGlu-Palm]-bhPhe-(D)Lys-C-NH₂ | | | |
| 7 | Isovaleric acid-D-T-H-[Dpa]-P-C-I- | | | |
| | (D)Lys-bhPhe-Lys[Ahx-Behenic_Acid]- | | | |
| | (D)Lys-C-NH₂ | | | |
| 8 | Isovaleric acid-D-T-H-[Dpa]-P-C-I- Lys[Ahx- | 550 | | |
| | Behenic_Acid]-bhPhe-(D)Lys-C-NH₂ | | | |
| 9 | Isovaleric acid-D-T-H-[Dpa]-P-C-I- | 18.39 | | |
| | Lys[1Peg2-1Peg2-isoGlu-Behenic_Acid]- | | | |
| | bhPhe-(D)Lys-C-NH₂ | | | |
| 10 | Isovaleric acid-D-T-H-[Dpa]-P-C-I- | 3.05 | | |
| | Lys[1Peg2-1Peg2-Ahx-C18_Diacid]-bhPhe- | | | |
| | K-(D)Lys-C-NH₂ | | | |
| 11 | Isovaleric acid-D-T-H-[Dpa]-P-C-I- | 12.72 | | |
| | Lys[1Peg2-1Peg2-Ahx-C18_Diacid]-bhPhe- | | | |
| | (D)Lys-C-NH₂ | | | |
| 12 | Isovaleric acid-D-T-H-[Dpa]-[Npc]-C-I- | 11.54 | | |
| | (D)Lys-bhPhe-[Lys(Ahx-Palm)]-C-(D)Lys- | | | |
| | NH₂ | | | |
| 13 | Isovaleric acid-D-T-H-[Dpa]-[Npc]-C-I- | 16.06 | | |
| | (D)Lys-bhPhe-Lys[isoGlu-Palm]-C-(D)Lys- | | | |
| | NH₂ | | | |
| 14 | Isovaleric acid-D-T-H-[Dpa]-[Npc]-C-I- | | | |
| | (D)Lys-bhPhe-Lys[Ahx-isoGlu-Palm]-C- | | | |
| | (D)Lys-NH₂ | | | |
| 15 | Isovaleric acid-D-T-H-[Dpa]-[Npc]-C-I- | >3000 | | |
| | (D)Lys-bhPhe-Lys[1Peg2-1Peg2-Ahx- | | | |
| | C18_Diacid]-C-(D)Lys-NH₂ | | | |
| 16 | Isovaleric acid-D-T-H-[Dpa]-P-C-I- | 5.85 | | |
| | (D)Lys-bhPhe-Lys[Palm]-K-C-NH₂ | | | |
| 17 | Isovaleric acid-D-T-H-[Dpa]-P-C-I- | 8.29 | | |
| | (D)Lys-bhPhe-[Lys(Ahx-Palm)]-K-C-NH₂ | | | |
| 18 | Isovaleric acid-D-T-H-[Dpa]-P-C-I-bhPhe- | 10.8 | | |
| | [Lys(Ahx-Palm)]-(D)Lys-C-NH₂ | | | |
| 19 | Isovaleric acid-D-T-H-[Dpa]-[Npc]-C-I- | 4.56 | | |
| | Lys[isoGlu-Palm]-bhPhe-C-(D)Lys-NH2 | | | |
| 20 | Isovaleric acid-D-T-H-[Dpa]-P-C-I- | 6.66 | | |
| | (D)Lys-bhPhe-[Lys(Ahx-Palm)]-(D)Lys-C- | | | |
| | NH₂ | | | |
| 21 | Isovaleric acid-D-T-H-[Dpa]-P-C-I- | 37.5 | | |
| | (D)Lys-bhPhe-[Lys(Ahx-Palm)]-[Spiral_Pip]- | | | |
| | C-NH₂ | | | |
| 22 | Isovaleric acid-D-T-H-[Dpa]-P-C-I- | 30.1 | | |
| | (D)Lys-bhPhe-[Lys(Ahx-Palm)]-Arg(Nitro)- | | | |
| | C-NH₂ | | | |
| 23 | Isovaleric acid-D-T-H-[Dpa]-P-C-I- | 41.6 | | |
| | (D)Lys-bhPhe-[Lys(Ahx-Palm)]- | | | |
| | Arg(Dimethyl)-C-NH₂ | | | |
| 24 | Isovaleric acid-D-T-H-[Dpa]-P-C-I- | 45.8 | | |
| | (D)Lys-bhPhe-[Lys(Ahx-Palm)]-Cit-C-NH₂ | | | |
| 25 | Isovaleric acid-D-T-H-[Dpa]-P-C-I- | 25.7 | | |
| | (D)Lys-bhPhe-[Lys(Ahx-Palm)]- Pro(4- | | | |
| | Amino)-C-NH₂ | | | |
| 26 | Isovaleric acid-D-T-H-[Dpa]-P-C-I- | 32.2 | | |
| | (D)Lys-bhPhe-[Lys(Ahx-Palm)]-Cav-C-NH₂ | | | |
| 27 | Isovaleric acid-D-T-H-[Dpa]-P-C-I- | 26.3 | | |
| | [Spiral_Pip]-bhPhe-[Lys(Ahx-Palm)]-(D)Lys- | | | |
| | C-NH₂ | | | |
| 28 | Isovaleric acid-D-T-H-[Dpa]-P-C-I- | 27.1 | | |
| | Arg(Nitro)-bhPhe-[Lys(Ahx-Palm)]-(D)Lys- | | | |
| | C-NH₂ | | | |
| 29 | Isovaleric acid-D-T-H-[Dpa]-P-C-I- | 10.6 | | |
| | Arg(Dimethyl)-bhPhe-[Lys(Ahx-Palm)]- | | | |
| | (D)Lys-C-NH₂ | | | |
| 30 | Isovaleric acid-D-T-H-[Dpa]-P-C-I-Cit- | 26.6 | | |
| | bhPhe-[Lys(Ahx-Palm)]-(D)Lys-C-NH2 | | | |
| 31 | Isovaleric acid-D-T-H-[Dpa]-P-C-I- | 18 | | |
| | Pro(4Amino)-bhPhe-[Lys(Ahx-Palm)]-(D)Lys- | | | |
| | C-NH₂ | | | |
| 32 | Isovaleric acid-D-T-H-[Dpa]-P-C-I-Cav- | | | |
| | bhPhe-[Lys(Ahx-Palm)]-(D)Lys-C-NH₂ | | | |
| 33 | Isovaleric Acid-D-T-H-[Dpa]-P-C-I- | 25.4 | | |
| | [(D)Lys]-[bhPhe]-[Lys(2Peg11′_Palm)]-P-R- | | | |
| | S-K-C-NH2 | | | |
| 34 | Isovaleric Acid-D-T-H-[Dpa]-P-C-I- | 23.6 | | |
| | [(D)Lys]-[bhPhe]-[Lys(2Peg11′_Palm)]-P-R- | | | |
| | S-K-[Sarc]-C-NH2 | | | |
| 35 | Isovaleric Acid-D-T-H-[Dpa]-P-C-I- | 23.1 | | |
| | [(D)Lys]-[bhPhe]-[Lys(2Peg11′_Palm)]-P- | | | |
| | [(D)Lys]-S-[(D)Lys]-[Sarc]-C-[(D)Lys]-NH2 | | | |
| 36 | Isovaleric Acid-D-T-H-[Dpa]-P-C-I- | 10.9 | | |
| | [(D)Lys]-[bhPhe]- | | | |
| | [Lys(2Peg11′_Sarc_Palm)]-[(D)Lys]-C-NH2 | | | |
| 37 | Isovaleric Acid-D-T-H-[Dpa]-P-C-I- | 19.6 | | |
| | [(D)Lys]-[bhPhe]- | | | |
| | [Lys(2Peg11′_Ahx_Palm)]-[(D)Lys]-C-NH2 | | | |
| 38 | Isovaleric Acid-D-T-H-[Dpa]-P-C-I- | 33 | | |
| | [(D)Lys]-[bhPhe]-[Lys(Lys[Peg11_Palm]2)]- | | | |
| | [(D)Lys]-C-NH2 | | | |
| 39 | Isovaleric Acid-D-T-H-[Dpa]-P-C-I- | 19.3 | | |
| | [(D)Lys]-[bhPhe]-[Lys(Ahx_Ahx_Palm)]- | | | |
| | [(D)Lys]-C-NH2 | | | |
| 40 | Isovaleric Acid-D-T-H-[Dpa]-P-C-I- | 18.5 | | |
| | [(D)Lys]-[bhPhe]- | | | |
| | [Lys(Ahx_Ahx_Ahx_Palm)]-[(D)Lys]-C- | | | |
| | NH2; | | | |
| 41 | Isovaleric Acid-D-T-H-[Dpa]-P-C-I- | 18.5 | | |
| | [(D)Lys]-[bhPhe]- | | | |
| | [Lys(2Peg11′_Peg11_Palm)]-[(D)Lys]-C- | | | |
| | NH2 | | | |
| 42 | Isovaleric Acid-D-T-H-[Dpa]-P-C-I- | 1700 | | |
| | [(D)Lys]-[bhPhe]- | | | |
| | [Lys(1Peg2_1Peg2_IsoGlu_C18_Diacid)]- | | | |
| | [(D)Lys]-C-NH2 | | | |
| 43 | Isovaleric Acid-D-T-H-[Dpa]-P-C-I- | 283 | | |
| | [(D)Lys]-[bhPhe]- | | | |
| | [Lys(1Peg2_1Peg2_Ahx_C18_Diacid)]- | | | |
| | [(D)Lys]-C-NH2 | | | |
| 44 | Isovaleric Acid-D-T-H-[Dpa]-P-C-I- | 37.9 | | |
| | [(D)Lys]-[bhPhe]- | | | |
| | [Lys(2Peg11′_KKK_Palm)]-[(D)Lys]-C-NH2 | | | |
| 45 | Isovaleric Acid-D-T-H-[Dpa]-P-C-I- | 24.5 | | |
| | [(D)Lys]-[bhPhe]- | | | |
| | [Lys(2Peg11′_KKKGS_Palm)]-[(D)Lys]-C- | | | |
| | NH2 | | | |

TABLE 3B
Illustrative Monomer Hepcidin Analogues

| Compd. ID/ | | FPN EC₅₀ |
|---|---|---|
| SEQ ID No. | Peptide | (nM) |
| 201 | Isovaleric Acid-D-T-H-[Dpa]-P-[Cys]-[MeLeu]-[(D)Lys]- | 12.5 |
| | [bhPhe]-[Lys(Ahx_Palm)]-[(D)Lys]-[Cys]-NH2 | |
| 202 | Isovaleric Acid-D-T-H-[Dpa]-P-[Cys]-I-[(D)Lys]-[MePhe]- | 16.2 |
| | [Lys(Ahx_Palm)]-[(D)Lys]-[Cys]-NH2 | |
| 203 | Isovaleric Acid-D-T-H-[MePhe]-P-[Cys]-I-[(D)Lys]-[bhPhe]- | 28 |
| | [Lys(Ahx_Palm)]-[(D)Lys]-[Cys]-NH2 | |
| 204 | Isovaleric Acid-[(D)Asp]-T-H-F-P-[Cys]-I- | 5.52 |
| | [Lys(IsoGlu_Palm)]-F-E-P-R-S-K-G-[Cys]-K-NH2 | |
| 205 | Isovaleric Acid-D-T-H-F-P-[Cys]-I-[(D)Lys]-[bhPhe]- | 4.23 |
| | [Lys(Palm)]-K-[Cys]-NH2 | |
| 206 | Isovaleric Acid-D-T-H-[Dpa]-P-[Cys]-I-[bhPhe]- | 10.8 |
| | [Lys(Ahx_Palm)]-[(D)Lys]-[Cys]-NH2 | |
| 207 | Isovaleric Acid-D-T-H-F-P-[Cys]-I-[Lys(IsoGlu_Palm)]-F-E- | 53.9 |
| | P-R-S-K-G-[Cys]-[Lys(Alexa647)]-NH2 | |
| 208 | Isovaleric Acid-D-T-H-F-P-[Cys]-I-[Lys(IsoGlu_Palm)]-F-E- | 21.1 |
| | P-R-S-[Lys(PEG12_Biotin)]-G-[Cys]-K-NH2 | |
| 209 | Isovaleric Acid-D-T-H-F-P-[Cys]-I-[Lys(IsoGlu_Palm)]-F-E- | |
| | P-R-S-[Lys(Ahx_Biotin)]-G-[Cys]-K-NH2 | |
| 210 | Isovaleric Acid-D-T-H-F-P-[Cys]-I-K-F- | 27.8 |
| | [Lys(1PEG2_1PEG2_Ahx_C18_Diacid)]-P-R-S-K-[Sarc]- | |
| | [Cys]-K-NH2 | |
| 211 | Isovaleric Acid-D-T-H-F-P-[Cys]-I-K-F- | 9.21 |
| | [Lys(PEG12_Sarc_Palm)]-P-R-S-K-[Sarc]-[Cys]-K-NH2 | |
| 212 | Isovaleric Acid-D-T-H-[Dpa]-P-[Cys]-I-K-F- | 20.7 |
| | [Lys(PEG12_Palm)]-P-R-S-K-[Sarc]-[Cys]-K-NH2 | |
| 213 | Isovaleric Acid-D-T-H-F-P-[Cys]-I-[Lys(Ac)]-F- | 11.1 |
| | [Lys(PEG12_Palm)]-P-R-S-[Lys(Ac)]-[Sarc]-[Cys]-K-NH2 | |
| 214 | Isovaleric Acid-D-T-H-F-P-[Cys]-I-[Lys(Ac)]-F- | 7.19 |
| | [Lys(PEG12_Palm)]-P-[Lys(Ac)]-S-[Lys(Ac)]-[Sarc]-[Cys]- | |
| | K-NH2 | |
| 215 | Isovaleric Acid-D-T-H-F-P-[Cys]-I-[Lys(IsoGlu)]-F-E-P-R-S- | |
| | K-G-[Cys]-K-NH2 | |
| 216 | Isovaleric Acid-D-T-H-F-P-[Cys]-L-[Lys(IsoGlu_Palm)]-F-E- | 16.6 |
| | P-R-S-K-G-[Cys]-K-NH2 | |
| 217 | Isovaleric Acid-D-T-H-F-P-CMe-I-[Lys(IsoGlu_Palm)]-F-E- | |
| | P-R-S-K-G-CMe-K-NH2 | |
| 218 | Isovaleric Acid-D-T-H-F-P-[Cys]-I-[Lys(IsoGlu_Palm)]-F-E- | 15.6 |
| | P-R-S-K-G-[Cys] | |
| 219 | Isovaleric Acid-D-T-H-F-P-C-I-[Lys(IsoGlu_Palm)]-F-E-P-R | 12.6 |
| 220 | Isovaleric Acid-D-T-H-F-P-[Cys]-I-[Lys(IsoGlu_Palm)]-F-E- | |
| | P-R-S-K-G-[Cys]-[Lys(Ac)]-NH2 | |
| 221 | Isovaleric Acid-D-T-H-F-P-[Cys]-I-[Lys(IsoGlu_Palm)]-F-E- | |
| | P-R-S-[Lys(Ac)]-G-[Cys]-K-NH2 | |
| 222 | Isovaleric Acid-D-T-H-F-P-[Cys]-I-[Lys(IsoGlu_Palm)]-F-E- | |
| | P-[Lys(Ac)]-S-K-G-[Cys]-K-NH2 | |
| 223 | Isovaleric Acid-D-T-H-F-P-[Cys]-I-[Lys(IsoGlu_Palm)]-F-E- | |
| | P-R-S-[Lys(Ac)]-G-[Cys]-[Lys(Ac)]-NH2 | |
| 224 | Isovaleric Acid-D-T-H-F-P-[Cys]-I-[Lys(IsoGlu_Palm)]-F-E- | |
| | P-[Lys(Ac)]-S-K-G-[Cys]-[Lys(Ac)]-NH2 | |
| 225 | Isovaleric Acid-D-T-H-F-P-[Cys]-I-[Lys(IsoGlu_Palm)]-F-E- | |
| | P-[Lys(Ac)]-S-[Lys(Ac)]-G-[Cys]-K-NH2 | |
| 226 | Isovaleric Acid-D-T-H-F-P-[Cys]-I-[Lys(IsoGlu_Palm)]-F-E- | |
| | P-[Lys(Ac)]-S-[Lys(Ac)]-G-[Cys]-[Lys(Ac)]-NH2 | |
| 227 | Isovaleric Acid-D-T-H-F-P-[Cys]-I-[Lys(IsoGlu_Ac)]-F-E-P- | |
| | R-S-K-G-[Cys]-K-NH2 | |
| 228 | Isovaleric Acid-D-T-H-F-P-[Cys]-I-[Lys(IsoGlu_Ac)]-F-E-P- | |
| | R-S-K-G-[Cys]-[Lys(Ac)]-NH2 | |
| 229 | Isovaleric Acid-D-T-H-F-P-[Cys]-I-[Lys(IsoGlu_Ac)]-F-E-P- | |
| | R-S-[Lys(Ac)]-G-[Cys]-K-NH2 | |
| 230 | Isovaleric Acid-D-T-H-F-P-[Cys]-I-[Lys(IsoGlu_Ac)]-F-E-P- | |
| | [Lys(Ac)]-S-K-G-[Cys]-K-NH2 | |
| 231 | Isovaleric Acid-D-T-H-F-P-[Cys]-I-[Lys(IsoGlu_Ac)]-F-E-P- | |
| | R-S-[Lys(Ac)]-G-[Cys]-[Lys(Ac)]-NH2 | |
| 232 | Isovaleric Acid-D-T-H-F-P-[Cys]-I-[Lys(IsoGlu_Ac)]-F-E-P- | |
| | [Lys(Ac)]-S-K-G-[Cys]-[Lys(Ac)]-NH2 | |
| 233 | Isovaleric Acid-D-T-H-F-P-[Cys]-I-[Lys(IsoGlu_Ac)]-F-E-P- | |
| | [Lys(Ac)]-S-[Lys(Ac)]-G-[Cys]-K-NH2 | |
| 234 | Isovaleric Acid-D-T-H-F-P-[Cys]-I-[Lys(IsoGlu_Ac)]-F-E-P- | |
| | [Lys(Ac)]-S-[Lys(Ac)]-G-[Cys]-[Lys(Ac)]-NH2 | |
| 235 | Isovaleric Acid-D-T-H-F-P-[Cys]-I-[Lys(IsoGlu_Palm)]-F-E- | 10.3 |
| | P-R-S-K-G-[Cys]-[MeLys]-NH2 | |
| 236 | Isovaleric Acid-D-T-H-F-P-[Cys]-I-[Lys(IsoGlu_Palm)]-F-E- | 16 |
| | P-R-S-K-G-me[Cys]-K-NH2 | |
| 237 | Isovaleric Acid-D-T-H-F-P-[Cys]-I-[Lys(IsoGlu_Palm)]-F-E- | 11.4 |
| | P-R-S-K-[Sarc]-[Cys]-K-NH2 | |
| 238 | Isovaleric Acid-D-T-H-F-P-[Cys]-I-[Lys(IsoGlu_Palm)]-F-E- | 10.6 |
| | P-R-S-[MeLys]-G-[Cys]-K-NH2 | |
| 239 | Isovaleric Acid-D-T-H-F-P-[Cys]-I-[Lys(IsoGlu_Palm)]-F-E- | 6.34 |
| | P-R-[MeSer]-K-G-[Cys]-K-NH2 | |
| 240 | Isovaleric Acid-D-T-H-F-P-[Cys]-I-[Lys(IsoGlu_Palm)]-F-E- | 11.1 |
| | P-[MeArg]-S-K-G-[Cys]-K-NH2 | |
| 241 | Isovaleric Acid-D-T-H-F-P-[Cys]-I-[Lys(IsoGlu_Palm)]-F- | 20.8 |
| | [MeGlu]-P-R-S-K-G-[Cys]-K-NH2 | |
| 242 | Isovaleric Acid-D-T-H-F-P-[Cys]-I-[Lys(IsoGlu_Palm)]- | 33.3 |
| | [MePhe]-E-P-R-S-K-G-[Cys]-K-NH2 | |
| 243 | Isovaleric Acid-D-T-H-F-P-[Cys]-I-me[Lys(IsoGlu_Palm)]-F- | 7.76 |
| | E-P-R-S-K-G-[Cys]-K-NH2 | |
| 244 | Isovaleric Acid-D-T-H-F-P-[Cys]-[MeLeu]- | 9.59 |
| | [Lys(IsoGlu_Palm)]-F-E-P-R-S-K-G-[Cys]-K-NH2 | |
| 245 | Isovaleric Acid-D-T-H-F-P-me[Cys]-I-[Lys(IsoGlu_Palm)]-F- | 16 |
| | E-P-R-S-K-G-[Cys]-K-NH2 | |
| 246 | Isovaleric Acid-D-T-H-F-P-[Cys]-I- | 332 |
| | [Lys(IsoGlu_PEG12_Biotin)]-F-E-P-R-S-K-G-[Cys]-K-NH2 | |
| 247 | Isovaleric Acid-D-T-H-F-P-[Cys]-I- | |
| | [Lys(IsoGlu_PEG3_Mal)]-F-E-P-R-S-K-G-[Cys]-K-NH2 | |
| 248 | Isovaleric Acid-D-T-H-F-dHyp_4S-[Cys]-I- | |
| | [Lys(IsoGlu_Palm)]-F-E-P-R-S-K-G-[Cys]-K-NH2 | |
| 249 | Isovaleric Acid-D-T-H-F-P-[Cys]-I-[Lys(IsoGlu_Palm)]-F-E- | |
| | [(D)Hydroxy Pro(4S)]-R-S-K-G-[Cys]-K-NH2 | |
| 250 | Isovaleric Acid-D-T-H-F-[HydroxyPro(4S)]-[Cys]-I- | |
| | [Lys(IsoGlu_Palm)]-F-E-P-R-S-K-G-[Cys]-K-NH2 | |
| 251 | Isovaleric Acid-D-T-H-F-P-[Cys]-I-[Lys(IsoGlu_Palm)]-F-E- | |
| | [Hydroxy Pro(4S)]-R-S-K-G-[Cys]-K-NH2 | |
| 252 | Isovaleric Acid-D-T-H-F-P-[Cys]-I- | |
| | [Lys(IsoGlu_HydroxyPalmSR)]-F-E-P-R-S-K-G-[Cys]-K- | |
| | NH2 | |
| 253 | Isovaleric Acid-D-T-H-[Dpa]-P-[Cys]-I- | >3000 |
| | [(D)Lys(PEG12_OMe)]-[bhPhe]- | |
| | [Lys(1PEG2_1PEG2_IsoGlu_C18_Diacid)]- | |
| | [(D)Lys(PEG12_OMe)]-[Cys]-NH2 | |
| 254 | Isovaleric Acid-D-T-H-F-[HydroxyPro(3S)]-[Cys]-I- | |
| | [Lys(IsoGlu_Palm)]-F-E-P-R-S-K-G-[Cys]-K-NH2 | |
| 255 | Isovaleric Acid-D-T-H-F-P-[Cys]-I-[Lys(IsoGlu_Palm)]-F-E- | |
| | [Hydroxy Pro(3S)]-R-S-K-G-[Cys]-K-NH2 | |
| 256 | Isovaleric Acid-D-T-H-F-[HydroxyPro(3R)]-[Cys]-I- | |
| | [Lys(IsoGlu_Palm)]-F-E-P-R-S-K-G-[Cys]-K-NH2 | |
| 257 | Isovaleric Acid-D-T-H-F-P-[Cys]-I-[Lys(IsoGlu_Palm)]-F-E- | |
| | [Hydroxy Pro(3R)]-R-S-K-G-[Cys]-K-NH2 | |
| 258 | Isovaleric Acid-D-T-H-F-P-[Cys]-I-[Lys(IsoGlu_Palm_OH)]- | |
| | F-E-P-R-S-K-G-[Cys]-K-NH2 | |

TABLE 3C
Illustrative Monomer Hepcidin Analogues

| | | IC50: FPN |
|---|---|---|
| Seq. | | internalisation |
| No. | Sequence | (nM) |
| 301 | [Isovaleric Acid]-D-T-H-F-P-C-I-[Lys(IsoGlu_palm)]-F-E-P-R- | |
| | S-K-G-C-[Lys(AFDye647)]-NH2 | |
| 302 | [Isovaleric Acid]-D-T-H-F-P-C-I-[Lys(IsoGlu_16F3Palm)]-F-E- | |
| | P-R-S-K-G-C-K-NH2 | |
| 303 | [Isovaleric Acid]-D-T-H-F-P-C-I-[Lys(IsoGlu_15OHPalm)]-F-E- | |
| | P-R-S-K-G-C-K-NH2 | |
| 304 | [Isovaleric Acid]-D-T-H-F-P-C-I-[Lys(IsoGlu_14OHPalm)]-F-E- | |
| | P-R-S-K-G-C-K-NH2 | |
| 305 | [Isovaleric Acid]-D-T-H-F-P-C-I-[Lys(IsoGlu_12OHPalm)]-F-E- | |
| | P-R-S-K-G-C-K-NH2 | |
| 306 | [Isovaleric Acid]-D-T-H-F-P-C-I-[Lys(IsoGlu_11OHPalm)]-F-E- | |
| | P-R-S-K-G-C-K-NH2 | |
| 307 | [Isovaleric Acid]-D-T-H-F-P-C-I-[Lys(IsoGlu_10OHPalm)]-F-E- | |
| | P-R-S-K-G-C-K-NH2 | |
| 308 | [Isovaleric Acid]-D-T-H-F-P-C-I-[Lys(IsoGlu_9OHPalm)]-F-E- | |
| | P-R-S-K-G-C-K-NH2 | |
| 309 | [Isovaleric Acid]-D-T-H-F-P-C-I-[Lys(IsoGlu_7OHPalm)]-F-E- | |
| | P-R-S-K-G-C-K-NH2 | |
| 310 | [Isovaleric Acid]-D-T-H-F-P-C-I-[Lys(IsoGlu_5OHPalm)]-F-E- | |
| | P-R-S-K-G-C-K-NH2 | |
| 311 | [Isovaleric Acid]-D-T-H-F-P-C-I-[Lys(IsoGlu_4OHPalm)]-F-E- | |
| | P-R-S-K-G-C-K-NH2 | |
| 312 | [Isovaleric Acid]-D-T-H-F-P-C-I-[Lys(IsoGlu_2OHPalm)]-F-E- | |
| | P-R-S-K-G-C-K-NH2 | |
| 313 | [Isovaleric Acid]-D-T-H-F-P-C-I-[Lys(IsoGlu_8OHPalm)]-F-E- | |
| | P-R-S-K-G-C-K-NH2 | |
| 314 | [Isovaleric Acid]-D-T-H-F-P-C-I-[Lys(IsoGlu_13OHPalm)]-F-E- | |
| | P-R-S-K-G-C-K-NH2 | |
| 315 | [Isovaleric Acid]-D-T-H-F-P-C-I-[Lys(IsoGlu_6OHPalm)]-F-E- | |
| | P-R-S-K-G-C-K-NH2 | |
| 316 | [Isovaleric Acid]-D-T-H-F-P-C-I-[Lys(IsoGlu_Palm)]_OH- | |
| | [Phe(13C9_15N)]-E-P-R-S-K-G-C-K-NH2 | |
| 317 | [Isovaleric Acid]-D-T-H-F-P-[Cys(SKGCKam)]-I- | 13.7 |
| | [Lys(IsoGlu_palm)]-F-E-P-R | |
| 318 | [Isovaleric Acid]-D-T-H-F-P-[Cys(Ckam)]-I- | |
| | [Lys(IsoGlu_palm)]-F-E-P-R-S-K-G | |
| 319 | [Isovaleric Acid]-D-T-H-F-P-C-I-[Lys(IsoGlu_palm)]-F-E-P-R- | 35.8 |
| | S-K-G-C-K-NH2 | |
| 320 | [Isovaleric Acid]-D-T-H-[Dpa]-P-C-I-[Lys(Ahx_Palm)]-[2Pal]- | 9.72 |
| | C-NH2 | |
| 321 | [Isovaleric Acid]-D-T-H-[Dpa]-P-C-I-[Lys(Ahx_Palm)]-[2Pal]- | 21.7 |
| | [(D)Lys]-C-NH2 | |
| 322 | [Isovaleric Acid]-D-T-H-[Dpa]-P-C-I-[(D)Lys]-[2Pal]- | |
| | [Lys(Ahx_Palm)]-[(D)Lys]-C-NH2 | |
| 323 | [Isovaleric Acid]-D-T-H-[Dpa]-P-C-I-[bAla]-[bhPhe]-[(D)Lys]- | 10.6 |
| | C-NH2 | |
| 324 | [Isovaleric Acid]-D-T-H-[Dpa]-P-C-I-[bhPhe]-[(D)Lys]-C-NH2 | 23.3 |
| 325 | [Isovaleric Acid]-D-T-H-[Dpa]-P-C-I-[(D)Lys(Ac)]-[bhPhe]- | 37.8 |
| | [Lys(Ac)]-C-NH2 | |
| 326 | [Isovaleric Acid]-D-T-H-[Dpa]-P-C-I-[(D)Lys]-[bhPhe]- | 95.7 |
| | [Lys(Ac)]-C-NH2 | |
| 327 | [Isovaleric Acid]-D-T-H-[Dpa]-P-C-I-[(D)Lys]-[bhPhe]-K- | 142 |
| | [(D)Lys]-C-NH2 | |
| 328 | [Isovaleric Acid]-D-T-H-[Dpa]-[Npc]-C-I-[(D)Lys]-[Phe(4-tBu)]- | 16.5 |
| | [(D)Lys]-C-[Lys(PEG12_Palm)]-NH2 | |
| 329 | [Isovaleric Acid]-D-T-H-[Dpa]-P-C-I-[PEG2]-[bhPhe]- | |
| | [Lys(PEG12_Palm)]-[(D)Lys]-C-NH2 | |
| 330 | [Isovaleric Acid]-D-T-H-[Dpa]-P-C-I-[bhPhe]- | 6.93 |
| | [Lys(PEG12_Palm)]-[(D)Lys]-C-NH2 | |
| 331 | [Isovaleric Acid]-D-T-H-[Dpa]-P-C-I-I-[bhPhe]- | 24 |
| | [Lys(PEG12 Palm)]-[(D)Lys]-C-NH2 | |
| 332 | [Isovaleric Acid]-D-T-H-[Dpa]-P-C-I-(D)Ala-[bhPhe]- | 11 |
| | [Lys(PEG12_Palm)]-[(D)Lys]-C-NH2 | |
| 333 | [Isovaleric Acid]-D-T-H-[Dpa]-P-C-I-[bAla]-[bhPhe]- | 7.16 |
| | [Lys(PEG12 Palm)]-[(D)Lys]-C-NH2 | |
| 334 | [Isovaleric Acid]-D-T-H-F-P-C-I-[(D)Lys]-F- | 32 |
| | [Lys(PEG12_Palm)]-C-K-NH2 | |
| 335 | [Isovaleric Acid]-D-T-H-[Dpa]-P-C-I-[(D)Lys]-F- | 18 |
| | [Lys(PEG12 Palm)]-[Dap]-[Hcy]-NH2 | |
| 336 | [Isovaleric Acid]-D-T-H-[Dpa]-P-C-I-[(D)Lys]-F- | 49.3 |
| | [Lys(PEG12_Palm)]-[Dap]-[Pen]-NH2 | |
| 337 | [Isovaleric Acid]-D-T-H-[Dpa]-P-C-I-[(D)Lys]-F- | 19.5 |
| | [Lys(PEG12_Palm)]-[(D)Lys]-C-NH2 | |
| 338 | [Isovaleric Acid]-D-T-H-[Dpa]-P-C-I-[(D)Lys]-F- | 20.4 |
| | [Lys(PEG12 Palm)]-bhLys-C-NH2 | |
| 339 | [Isovaleric Acid]-D-T-H-[Dpa]-P-C-I-[(D)Lys]-F- | 22.8 |
| | [Lys(PEG12 Palm)]-[Dab]-C-NH2 | |
| 340 | [Isovaleric Acid]-D-T-H-[Dpa]-P-C-I-[(D)Lys]-F- | 26.9 |
| | [Lys(PEG12_Palm)]-[Orn]-C-NH2 | |
| 341 | [Isovaleric Acid]-D-T-H-[Dpa]-P-C-I-[(D)Lys]-F- | 22 |
| | [Lys(PEG12_Palm)]-C-NH2 | |
| 342 | [Isovaleric Acid]-D-T-H-[Dpa]-P-C-I-[(D)Lys]-F- | 21.8 |
| | Lys(PEG12_Palm)]-[(D)Lys]-[N-MeCys]-NH2 | |
| 343 | [Isovaleric Acid]-D-T-H-[Dpa]-P-C-I-[(D)Lys]-F- | 13.5 |
| | [Lys(PEG12_Palm)]-K-[N-MeCys]-NH2 | |
| 344 | [Isovaleric Acid]-D-T-H-[Dpa]-P-C-I-[(D)Lys]-F- | 7.75 |
| | [Lys(PEG12_Palm)]-[(D)Lys]-[Pen]-NH2 | |
| 345 | [Isovaleric Acid]-D-T-H-[Dpa]-P-C-I-[(D)Lys]-[Dpa]- | 23 |
| | [Lys(PEG12_Palm)]-[(D)Lys]-C-NH2 | |
| 346 | [Isovaleric Acid]-D-T-H-[Dpa]-[Npc]-C-I-[(D)Lys]-[Phe(3,4- | 6.16 |
| | diF)]-[Lys(PEG12 Palm)]-K-C-NH2 | |
| 347 | [Isovaleric Acid]-D-T-H-[Dpa]-[Npc]-C-I-[(D)Lys]-[Phe(4-CN)]- | 23 |
| | [Lys(PEG12_Palm)]-K-C-NH2 | |
| 348 | [Isovaleric Acid]-D-T-H-[Dpa]-[Npc]-C-I-[(D)Lys]-[aMePhe]- | 2.1 |
| | [Lys(PEG12 Palm)]-K-C-NH2 | |
| 349 | [Isovaleric Acid]-D-T-H-[Dpa]-P-C-I-[(D)Lys]-[bhPhe]- | 11.2 |
| | [Lys(PEG12_Palm)]-[(D)Lys]-C-NH2 | |
| 350 | [Isovaleric Acid]-D-T-H-[Dpa]-[bhPro]-C-I-[(D)Lys]-F- | 0.8 |
| | [Lys(PEG12_Palm)]-[(D)Lys]-C-NH2 | |
| 351 | [Isovaleric Acid]-D-T-H-F-[Npc]-C-I-[(D)Lys]-F- | 13.2 |
| | [Lys(PEG12_Palm)]-[(D)Lys]-C-NH2 | |
| 352 | [Isovaleric Acid]-D-T-H-[Dpa]-[Npc]-C-I-[(D)Lys]-F- | 6.48 |
| | [Lys(PEG12 Palm)]-[(D)Lys]-C-NH2 | |
| 353 | [Isovaleric Acid]-D-T-H-F-P-[aMeCys]-I-[(D)Lys]-F- | 21.5 |
| | [Lys(PEG12_Palm)]-K-C-NH2 | |
| 354 | [Isovaleric Acid]-D-T-H-F-P-[aMeCys]-I-[(D)Lys]-F- | 10 |
| | [Lys(PEG12 Palm)]-K-[aMeCys]-NH2 | |
| 355 | [Isovaleric Acid]-D-T-H-F-P-C-I-[(D)Lys]-F- | 7.55 |
| | [Lys(PEG12_Palm)]-K-[aMeCys]-NH2 | |
| 356 | [Isovaleric Acid]-D-T-H-[Dpa]-[Npc]-C-I-[(D)Lys]-[Phe(4- | 0.1 |
| | COOH)]-[Lys(PEG12_Palm)]-K-C-NH2 | |
| 357 | [Isovaleric Acid]-D-T-H-[Dpa]-[Npc]-C-I-[(D)Lys]-[2Nal]- | 10.2 |
| | [Lys(PEG12_Palm)]-K-C-NH2 | |
| 358 | [Isovaleric Acid]-D-T-H-[Dpa]-[Npc]-C-I-[(D)Lys]-[Phe(4-(2- | >3000 |
| | aminoethoxy)]-[Lys(PEG12 Palm)]-K-C-NH2 | |
| 359 | [Isovaleric Acid]-D-T-H-[Dpa]-[Npc]-C-I-[(D)Lys]-[Phe(4-tBu)]- | 8.57 |
| | [Lys(PEG12_Palm)]-K-C-NH2 | |
| 360 | [Isovaleric Acid]-D-T-H-[Dpa]-[Npc]-C-I-[(D)Lys]-F- | 9.64 |
| | [Lys(PEG12_Palm)]-K-C-NH2 | |

In certain embodiment, the present invention includes a hepcidin analogue having a structure or comprising an amino acid sequence set forth below:
Isovaleric acid-D-T-H-[Dpa]-P—C—I—[Dap(Dap-Palm)]-bhPhe-C—NH₂;
Isovaleric acid-D-T-H-[Dpa]-P—C-I-(D)Lys-bhPhe-[Lys(Ahx-Palm)]-R—C—NH₂;
Isovaleric acid-D-T-H-[Dpa]-P—C-I-(D)Lys-bhPhe-[Lys(Ahx-Palm)]-(D)Lys-meCys-NH₂;
Isovaleric acid-D-T-H-[Dpa]- [Npc]-C-I-(D)Lys-bhPhe-[Lys (Ahx-Palm)]-(D)Lys-C—NH₂;
Isovaleric acid-D-T-H-[Dpa]-P—C-I-Lys[Ahx-isoGlu-Palm]-bhPhe--(D)Lys-C—NH₂;
Isovaleric acid-D-T-H-[Dpa]-P—C-I-Lys[isoGlu-Palm]-bhPhe-(D)Lys-C—NH₂;
Isovaleric acid-D-T-H-[Dpa]-P—C-I-(D)Lys-bhPhe- K(Ahx-Behenic Acid)-(D)Lys-C—NH₂;
Isovaleric acid-D-T-H-[Dpa]-P—C-I-K(Ahx-Behenic Acid)-bhPhe-(D)Lys-C—NH₂;
Isovaleric acid-D-T-H-[Dpa]-P—C-I-K(1Peg2-1Peg2-isoGlu-Behenic Acid)-bhPhe-(D)Lys-C—NH₂;
Isovaleric acid-D-T-H-[Dpa]-P—C-I-Lys[1Peg2-1Peg2-Ahx-C18 acid]-bhPhe-K-(D)Lys-C—NH₂;
Isovaleric acid-D-T-H-[Dpa]-P—C-I-Lys[1Peg2-1Peg2-Ahx-C18 acid]-bhPhe-(D)Lys-C—NH₂;
Isovaleric acid-D-T-H-[Dpa]-[Npc]-C-I-(D)Lys-bhPhe-[Lys(Ahx-Palm)]-C-(D)Lys-NH₂;
Isovaleric acid-D-T-H-[Dpa]-[Npc]-C-I-(D)Lys-bhPhe-Lys[isoGlu-Palm]-C-(D)Lys-NH₂;
Isovaleric acid-D-T-H-[Dpa]-[Npc]-C-I-(D)Lys-bhPhe-Lys[Ahx-isoGlu-Palm]-C-(D)Lys-NH₂;
Isovaleric acid-D-T-H-[Dpa]-[Npc]-C-I-(D)Lys-bhPhe-Lys[1Peg2-1Peg2-Ahx-C18 acid]-C-(D)Lys-NH₂;
Isovaleric acid-D-T-H-[Dpa]-P—C-I-(D)Lys-bhPhe-Lys[Palm]-K—C—NH₂;
Isovaleric acid-D-T-H-[Dpa]-P—C-I-(D)Lys-bhPhe-[Lys(Ahx-Palm)]-K—C—NH₂;
Isovaleric acid-D-T-H-[Dpa]-P—C-I-bhPhe-[Lys(Ahx-Palm)]-(D)Lys-C—NH₂;
Isovaleric acid-D-T-H-[Dpa]-[Npc]-C-I-Lys[isoGlu-Palm]-bhPhe-C-(D)Lys-NH₂;
Isovaleric acid(2:1)-D(x,2:1, 3:3)-T(3:1)-H-[Dpa]-P—C-I-(D)Lys-bhPhe-[Lys(Ahx-Palm)]-(D)Lys-C—NH₂;
Isovaleric acid-D-T-H-[Dpa]-P—C-I-(D)Lys-bhPhe-[Lys(Ahx-Palm)]-[Spiro_Pip]-C—NH₂;
Isovaleric acid-D-T-H-[Dpa]-P—C-I-(D)Lys-bhPhe-[Lys(Ahx-Palm)]-Arg(Nitro)-C—NH₂;
Isovaleric acid-D-T-H-[Dpa]-P—C-I-(D)Lys-bhPhe-[Lys(Ahx-Palm)]-Arg(Dimethyl)-C—NH₂;
Isovaleric acid-D-T-H-[Dpa]-P—C-I-(D)Lys-bhPhe-[Lys(Ahx-Palm)]-Cit-C—NH₂;
Isovaleric acid-D-T-H-[Dpa]-P—C-I-(D)Lys-bhPhe-[Lys(Ahx-Palm)]- Pro(4Amino)-C—NH₂;
Isovaleric acid-D-T-H-[Dpa]-P—C-I-(D)Lys-bhPhe-[Lys(Ahx-Palm)]-Cav-C—NH₂;
Isovaleric acid-D-T-H-[Dpa]-P—C—I—[Spiro_Pip]-bhPhe-[Lys(Ahx-Palm)]-(D)Lys-C—NH₂;
Isovaleric acid-D-T-H-[Dpa]-P—C-I-Arg(Nitro)-bhPhe-[Lys(Ahx-Palm)]-(D)Lys-C—NH₂;
Isovaleric acid-D-T-H-[Dpa]-P—C-I-Arg(Dimethyl)-bhPhe-[Lys(Ahx-Palm)]-(D)Lys-C—NH₂;
Isovaleric acid-D-T-H-[Dpa]-P—C-I-Cit-bhPhe-[Lys(Ahx-Palm)]-(D)Lys-C—NH₂;
Isovaleric acid-D-T-H-[Dpa]-P—C-I-Pro(4-Amino)-bhPhe-[Lys(Ahx-Palm)]-(D)Lys-C—NH₂;
Isovaleric acid-D-T-H-[Dpa]-P—C-I-Cav-bhPhe-[Lys(Ahx-Palm)]-(D)Lys-C—NH₂; or
Isovaleric acid-D-T-H-[Dpa]-P—C-I-Lys[1Peg2-1Peg2-Ahx-C18 acid]-bhPhe-(D)Lys-C—NH₂;
   and wherein the peptide is optionally cyclized via a disulfide bond between two Cys.

In certain embodiment, the present invention includes a hepcidin analogue having a structure or comprising an amino acid sequence set forth below:

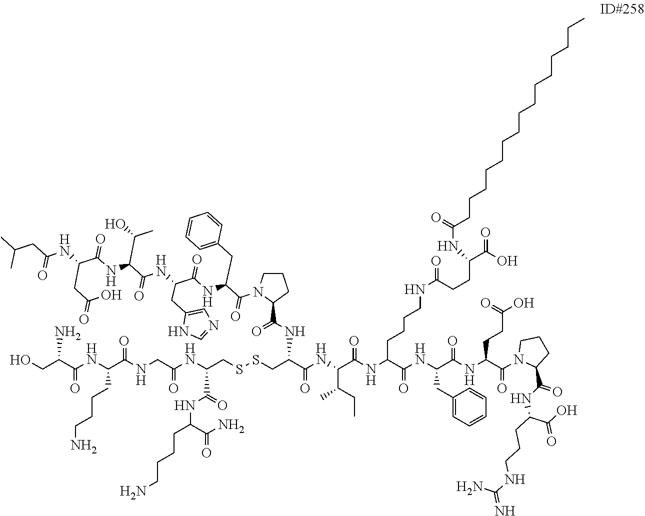 [SMILES: CCCCCCCCCCCCCCCC(=O)N[C@@H](CCC(=O)NCCCCC(NC(=O)[C@@H](NC(=O)[C@H](CSSC[C@@H](NC(=O)CNC(=O)[C@H](CCCCN)NC(=O)[C@@H](N)CO)C(=O)NC(CCCCN)C(N)=O)NC(=O)[C@@H]1CCCN1C(=O)[C@H](Cc1ccccc1)NC(=O)[C@H](Cc1cnc[nH]1)NC(=O)[C@@H](NC(=O)[C@H](CC(=O)O)NC(=O)CC(C)C)[C@@H](C)O)[C@@H](C)CC)C(=O)N[C@@H](Cc1ccccc1)C(=O)N[C@@H](CCC(=O)O)C(=O)N1CCC[C@H]1C(=O)N[C@@H](CCCNC(=N)N)C(=O)O)C(=O)O]

Isovaleric Acid-D-T-H-F-P-Cys(R′)-Ile-Lys(IsoGlu Palm)-F-E-P-R-OH, wherein R′ is H₂N—S-K-G-C-Lys-NH₂ and R′ is attached to Cys via a Cys-Cys disulfide bond.

In certain embodiment, the present invention provides a peptide or a peptide dimer thereof, wherein the peptide comprises or consists of any one of the peptides disclosed herein or listed in any of Tables 2, and 3. In one embodiment, the peptide comprises a disulfide bond between the two Cys, Cys and N-MeCys, or Cys and Pen residues. In a particular embodiment, the peptide is any one of peptides wherein the FPN activity is <100 nM. In another particular embodiment, the peptide is any one of peptides wherein the FPN activity is <50 nM. In another particular embodiment, the peptide is any one of peptides wherein the FPN activity is <20 nM. In another particular embodiment, the peptide is any one of peptides wherein the FPN activity is <10 nM. In more particular embodiment, the peptide is any one of peptides wherein the FPN activity is <5 nM.

In certain embodiment, the peptide is selected from a group of peptides listed in Tables 2 and 3, and wherein the SIF half life is >24 h.

### Peptide Analogue Conjugates

In certain embodiments, hepcidin analogues of the present invention, including both monomers and dimers, comprise one or more conjugated chemical substituents, such as lipophilic substituents and polymeric moieties, collectively referred to herein as half-life extension moieties. Without wishing to be bound by any particular theory, it is believed that the lipophilic substituent binds to albumin in the bloodstream, thereby shielding the hepcidin analogue from enzymatic degradation, and thus enhancing its half-life. In addition, it is believed that polymeric moieties enhance half-life and reduce clearance in the bloodstream, and in some cases enhance permeability through the epithelium and retention in the lamina propria. Moreover, it is also surmised that these substituents in some cases may enhance permeability through the epithelium and retention in the lamina propria. The skilled person will be well aware of suitable techniques for preparing the compounds employed in the context of the invention. For examples of non-limiting suitable chemistry, see, e.g., WO98/08871, WO00/55184, WO00/55119, Madsen et al (J. Med. Chem. 2007, 50, 6126-32), and Knudsen et al. 2000 (J. Med Chem. 43, 1664-1669).

In one embodiment, the side chains of one or more amino acid residues (e.g., Lys residues) in a hepcidin analogue of the invention is further conjugated (e.g., covalently attached) to a lipophilic substituent or other half-life extension moiety. The lipophilic substituent may be covalently bonded to an atom in the amino acid side chain, or alternatively may be conjugated to the amino acid side chain via one or more spacers or linker moieties. The spacer or linker moiety, when present, may provide spacing between the hepcidin analogue and the lipophilic substituent.

In certain embodiments, the lipophilic substituent or half-life extension moiety comprises a hydrocarbon chain having from 4 to 30 C atoms, for example at least 8 or 12 C atoms, and preferably 24 C atoms or fewer, or 20 C atoms or fewer. The hydrocarbon chain may be linear or branched and may be saturated or unsaturated. In certain embodiments, the hydrocarbon chain is substituted with a moiety which forms part of the attachment to the amino acid side chain or the spacer, for example an acyl group, a sulfonyl group, an N atom, an O atom or an S atom. In some embodiments, the hydrocarbon chain is substituted with an acyl group, and accordingly the hydrocarbon chain may form part of an alkanoyl group, for example palmitoyl, caproyl, lauroyl, myristoyl or stearoyl.

A lipophilic substituent may be conjugated to any amino acid side chain in a hepcidin analogue of the invention. In certain embodiment, the amino acid side chain includes a carboxy, hydroxyl, thiol, amide or amine group, for forming an ester, a sulphonyl ester, a thioester, an amide or a sulphonamide with the spacer or lipophilic substituent. For example, the lipophilic substituent may be conjugated to Asn, Asp, Glu, Gln, His, Lys, Arg, Ser, Thr, Tyr, Trp, Cys or Dbu, Dpr or Orn. In certain embodiments, the lipophilic substituent is conjugated to Lys. An amino acid shown as Lys in any of the formula provided herein may be replaced by, e.g., Dbu, Dpr or Om where a lipophilic substituent is added.

In further embodiments of the present invention, alternatively or additionally, the side-chains of one or more amino acid residues in a hepcidin analogue of the invention may be conjugated to a polymeric moiety or other half-life extension moiety, for example, in order to increase solubility and/or half-life in vivo (e.g., in plasma) and/or bioavailability. Such modifications are also known to reduce clearance (e.g. renal clearance) of therapeutic proteins and peptides.

As used herein, “Polyethylene glycol” or “PEG” is a polyether compound of general formula H—(O—CH₂—CH₂)ₙ—OH. PEGs are also known as polyethylene oxides (PEOs) or polyoxyethylenes (POEs), depending on their molecular weight PEO, PEE, or POG, as used herein, refers to an oligomer or polymer of ethylene oxide. The three names are chemically synonymous, but PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. Throughout this disclosure, the 3 names are used indistinguishably. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g., viscosity) due to chain length effects, their chemical properties are nearly identical. The polymeric moiety is preferably water-soluble (amphiphilic or hydrophilic), non-toxic, and pharmaceutically inert. Suitable polymeric moieties include polyethylene glycols (PEG), homo- or co-polymers of PEG, a monomethyl-substituted polymer of PEG (mPEG), or polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992). Also encompassed are PEGs that are prepared for purpose of half-life extension, for example, mono-activated, alkoxy-terminated polyalkylene oxides (POA's) such as mono-methoxy-terminated polyethyelene glycols (mPEG's); bis activated polyethylene oxides (glycols) or other PEG derivatives are also contemplated. Suitable polymers will vary substantially by weights ranging from about 200 to about 40,000 are usually selected for the purposes of the present invention. In certain embodiments, PEGs having molecular weights from 200 to 2,000 daltons or from 200 to 500 daltons are used. Different forms of PEG may also be used, depending on the initiator used for the polymerization process, e.g., a common initiator is a monofunctional methyl ether PEG, or methoxypoly(ethylene glycol), abbreviated mPEG. Other suitable initiators are known in the art and are suitable for use in the present invention.

Lower-molecular-weight PEGs are also available as pure oligomers, referred to as monodisperse, uniform, or discrete. These are used in certain embodiments of the present invention.

PEGs are also available with different geometries: branched PEGs have three to ten PEG chains emanating from a central core group; star PEGs have 10 to 100 PEG chains emanating from a central core group; and comb PEGs have multiple PEG chains normally grafted onto a polymer backbone. PEGs can also be linear. The numbers that are often included in the names of PEGs indicate their average molecular weights (e.g. a PEG with n=9 would have an average molecular weight of approximately 400 daltons, and would be labeled PEG 400.

As used herein, “PEGylation” is the act of coupling (e.g., covalently) a PEG structure to the hepcidin analogue of the invention, which is in certain embodiments referred to as a “PEGylated hepcidin analogue”. In certain embodiments, the PEG of the PEGylated side chain is a PEG with a molecular weight from about 200 to about 40,000. In certain embodiments, the PEG portion of the conjugated half-life extension moiety is PEG3, PEG4, PEG5, PEG6, PEG7, PEG8, PEG9, PEG10, or PEG11. In particular embodiments, it is PEG11. In certain embodiments, the PEG of a PEGylated spacer is PEG3 or PEG8. In some embodiments, a spacer is PEGylated. In certain embodiments, the PEG of a PEGylated spacer is PEG3, PEG4, PEG5, PEG6, PEG7, PEG8, PEG9, PEG10, or PEG11. In certain embodiments, the PEG of a PEGylated spacer is PEG3 or PEG8.

In some embodiments, the present invention includes a hepcidin analogue peptide (or a dimer thereof) conjugated with a PEG that is attached covalently, e.g., through an amide, a thiol, via click chemistry, or via any other suitable means known in the art. In particular embodiments PEG is attached through an amide bond and, as such, certain PEG derivatives used will be appropriately functionalized. For example, in certain embodiments, PEG11, which is O-(2-aminoethyl)-O′-(2-carboxyethyl)-undecaethyleneglycol, has both an amine and carboxylic acid for attachment to a peptide of the present invention. In certain embodiments, PEG25 contains a diacid and 25 glycol moieties.

Other suitable polymeric moieties include poly-amino acids such as poly-lysine, poly-aspartic acid and poly-glutamic acid (see for example Gombotz, et al. (1995), Bioconjugate Chem., vol. 6: 332-351; Hudecz, et al. (1992), Bioconjugate Chem., vol. 3, 49-57 and Tsukada, et al. (1984), J. Natl. Cancer Inst., vol. 73,: 721-729. The polymeric moiety may be straight-chain or branched. In some embodiments, it has a molecular weight of 500-40,000 Da, for example 500-10,000 Da, 1000-5000 Da, 10,000-20,000 Da, or 20,000-40,000 Da.

In some embodiments, a hepcidin analogue of the invention may comprise two or more such polymeric moieties, in which case the total molecular weight of all such moieties will generally fall within the ranges provided above.

In some embodiments, the polymeric moiety may be coupled (by covalent linkage) to an amino, carboxyl or thiol group of an amino acid side chain. Certain examples are the thiol group of Cys residues and the epsilon amino group of Lys residues, and the carboxyl groups of Asp and Glu residues may also be involved.

The skilled worker will be well aware of suitable techniques which can be used to perform the coupling reaction. For example, a PEG moiety bearing a methoxy group can be coupled to a Cys thiol group by a maleimido linkage using reagents commercially available from Nektar Therapeutics AL. See also WO 2008/101017, and the references cited above, for details of suitable chemistry. A maleimide-functionalised PEG may also be conjugated to the side-chain sulfhydryl group of a Cys residue.

As used herein, disulfide bond oxidation can occur within a single step or is a two-step process. As used herein, for a single oxidation step, the trityl protecting group is often employed during assembly, allowing deprotection during cleavage, followed by solution oxidation. When a second disulfide bond is required, one has the option of native or selective oxidation. For selective oxidation requiring orthogonal protecting groups, Acm and Trityl is used as the protecting groups for cysteine. Cleavage results in the removal of one protecting pair of cysteine allowing oxidation of this pair. The second oxidative deprotection step of the cysteine protected Acm group is then performed. For native oxidation, the trityl protecting group is used for all cysteines, allowing for natural folding of the peptide.

A skilled worker will be well aware of suitable techniques which can be used to perform the oxidation step.

In particular embodiments, a hepcidin analogue of the present invention comprises a half-life extension moiety, which may be selected from but is not limited to the following: Ahx-Palm, PEG2-Palm, PEG11-Palm, isoGlu-Palm, dapa-Palm, isoGlu-Lauric acid, isoGlu-Mysteric acid, and isoGlu-Isovaleric acid.

In particular embodiments, a hepcidin analogue comprises a half-life extension moiety having the structure shown below, wherein n=0 to 24 or n=14 to 24:

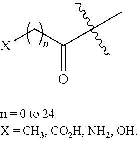

In certain embodiments, a hepcidin analogue of the present invention comprises a conjugated half-life extension moiety shown in Table 4.

TABLE 4
Illustrative Half-Life Extension Moieties

| # | Conjugates |
|---|---|
| C1 | 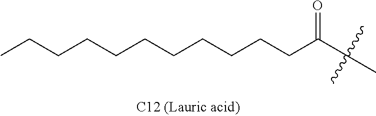 [SMILES: *C(=O)CCCCCCCCCCC] |
| C2 | 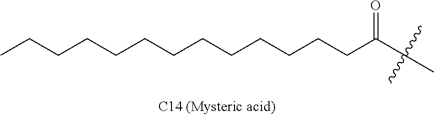 [SMILES: *C(=O)CCCCCCCCCCCCC] |
| C3 | 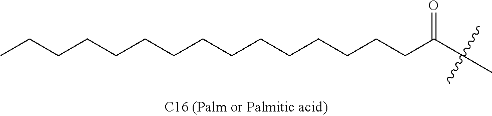 [SMILES: *C(=O)CCCCCCCCCCCCCCC] |
| C4 | 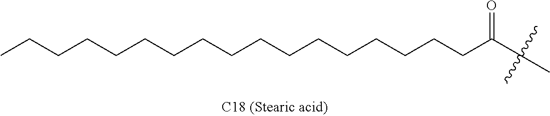 [SMILES: *C(=O)CCCCCCCCCCCCCCCCC] |
| C5 | 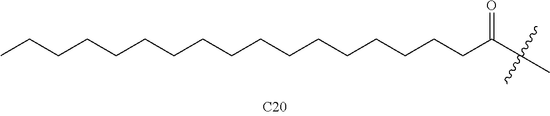 [SMILES: *C(=O)CCCCCCCCCCCCCCCCC] |
| C6 | 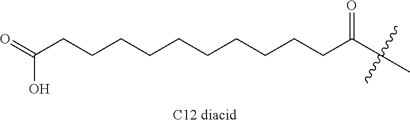 [SMILES: *C(=O)CCCCCCCCCCC(=O)O] |
| C7 | 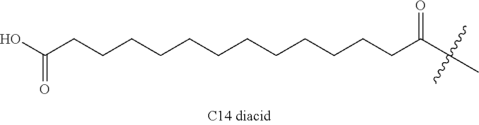 [SMILES: *C(=O)CCCCCCCCCCCCC(=O)O] |
| C8 | 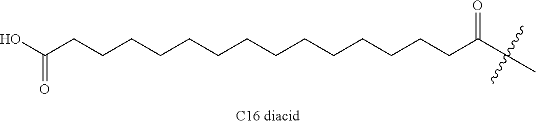 [SMILES: *C(=O)CCCCCCCCCCCCCCC(=O)O] . |
| C9 | 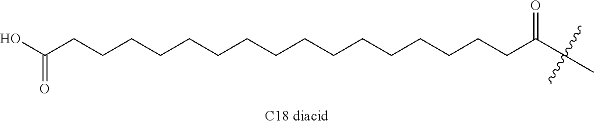 [SMILES: *C(=O)CCCCCCCCCCCCCCCCC(=O)O] |
| C10 | 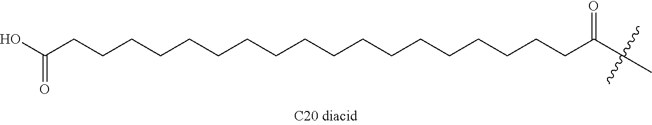 [SMILES: *C(=O)CCCCCCCCCCCCCCCCCCC(=O)O] |
| C11 | 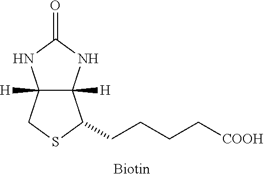 [SMILES: O=C(O)CCCC[C@@H]1SC[C@@H]2NC(=O)N[C@@H]21] |
| C12 | 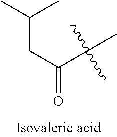 [SMILES: *C(=O)CC(C)C] |

In certain embodiments, a half-life extension moiety is conjugated directly to a hepcidin analogue, while in other embodiments, a half-life extension moiety is conjugated to a hepcidin analogue peptide via a linker moiety e.g., any of those depicted in Table 5.

TABLE 5
Illustrative Linker Moieties

| # | Linker Moiety |
|---|---|
| L1 | 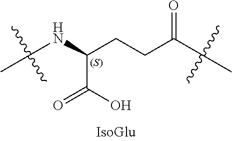 |
| L2 | 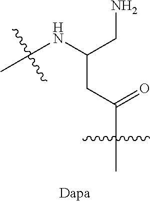 [SMILES: *NC(CN)CC(*)=O] |
| L3 | 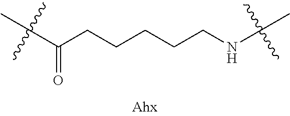 [SMILES: *NCCCCCC(*)=O] |
| | Lipdic gased linkers: |
| L4 | 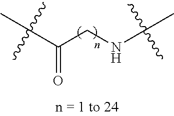 |
| L5 | 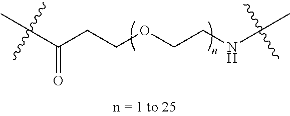 |
| | —[C(O)CH₂CH₂)OCH₂)ₙN(H)]-PEG based linkers (n- 5-25)PEG based linkers |
| L6 | 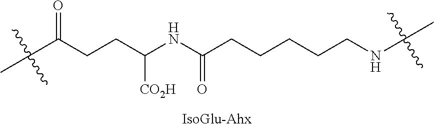 [SMILES: *NCCCCCC(=O)NC(CCC(*)=O)C(=O)O] |
| L7 | —[C(O)—CH2—(Peg)2—NH]— or —[C(O)—CH₂—(OCH₂CH₂)₂—NH]— (1Peg2) |
| L8 | -[(C(O)—CH2—(OCH2CH2)2—NH-C(O)—CH2—(OCH2CH2)2—NH-]-(1Peg2-1Peg2) |
| L9 | —[C(O)—CH2—CH2—(Peg)2—NH]— or —[C(O)—CH2—CH2—(OCH2CH2)2 —NH] - (2Peg2) |
| L10 | —[C(O)—CH2—CH2—(Peg)4—NH]— or —[C(O)—CH2—CH2—(OCH2CH2)4—NH]— - (2Peg4) |
| L11 | —[C(O)—CH2—(Peg)8—NH]— or —[C(O)—CH2—(OCH2CH2)8—NH]— (1Peg8) |
| L12 | —[C(O)—CH2—CH2—(Peg)8—NH]— or —[C(O)—CH2—CH2—(OCH2CH2)8— NH]— (2Peg8) |
| L17 | —[C(O)—CH2—(Peg)11—NH]— or —[C(O)—CH2—(OCH2CH2)11—NH]— (1Peg11) |
| L18 | —[C(O)—CH2—CH2—(Peg)11—NH]— or —[C(O)—CH2—CH2—(OCH2CH2)11—NH]— (2Peg11) |
| L19 | —[C(O)—CH2—CH2—(Peg)12—NH]— or —[C(O)—CH2—CH2—(OCH2CH2)12—NH]— (2Peg11′ or 2Peg12) |

With reference to linker structures shown in Table 5, reference to n=1 to 24 or n=1 to 25, or the like, (e.g., in L4, or L5) indicates that n may be any integer within the recited range. Additional linker moieties can be used are shown in “Abbreviation” table.

In particular embodiments, a hepcidin analogue of the present invention comprises any of the linker moieties shown in Table 5 and any of the half-life extension moieties shown in Table 4, including any of the following combinations shown in Table 6.

TABLE 6
Illustrative Combinations of Linkers and Half-
Life Extension Moieties in Hepcidin Analogues

| | Half-Life | | Half-Life | | Half-Life |
|---|---|---|---|---|---|
| | Extension | | Extension | | Extension |
| Linker | Moiety | Linker | Moiety | Linker | Moiety |
| L1 | C1 | L1 | C2 | L1 | C3 |
| L2 | C1 | L2 | C2 | L2 | C3 |
| L3 | C1 | L3 | C2 | L3 | C3 |
| L4 | C1 | LA | C2 | L4 | C3 |
| L5 | C1 | L5 | C2 | L5 | C3 |
| L6 | C1 | L6 | C2 | L6 | C3 |
| L7 | C1 | L7 | C2 | L7 | C3 |
| L8 | C1 | L8 | C2 | L8 | C3 |
| L9 | C1 | L9 | C2 | L9 | C3 |
| L10 | C1 | L10 | C2 | L10 | C3 |
| L11 | C1 | L11 | C2 | L11 | C3 |
| L12 | C1 | L12 | C2 | L12 | C3 |
| L13 | C1 | L13 | C2 | L13 | C3 |
| L14 | C1 | L14 | C2 | L14 | C3 |
| L15 | C1 | L15 | C2 | L15 | C3 |
| L16 | C1 | L16 | C2 | L16 | C3 |
| L17 | C1 | L17 | C2 | L17 | C3 |
| L18 | C1 | L18 | C2 | L18 | C3 |
| L19 | C1 | L19 | C2 | L19 | C3 |
| L1 | C4 | L1 | C5 | L1 | C6 |
| L2 | C4 | L2 | C5 | L2 | C6 |
| L3 | C4 | L3 | C5 | L3 | C6 |
| L4 | C4 | L4 | C5 | L4 | C6 |
| L5 | C4 | L5 | C5 | L5 | C6 |
| L6 | C4 | L6 | C5 | L6 | C6 |
| L7 | C4 | L7 | C5 | L7 | C6 |
| L8 | C4 | L8 | C5 | L8 | C6 |
| L9 | C4 | L9 | C5 | L9 | C6 |
| L10 | C4 | L10 | C5 | L10 | C6 |
| L11 | C4 | L11 | C5 | L11 | C6 |
| L12 | C4 | L12 | C5 | L12 | C6 |
| L13 | C4 | L13 | C5 | L13 | C6 |
| L14 | C4 | L14 | C5 | L14 | C6 |
| L15 | C4 | L15 | C5 | L15 | C6 |
| L16 | C4 | L16 | C5 | L16 | C6 |
| L17 | C4 | L17 | C5 | L17 | C6 |
| L18 | C4 | L18 | C5 | L18 | C6 |
| L19 | C4 | L19 | C5 | L19 | C6 |
| L1 | C7 | L1 | C8 | L1 | C9 |
| L2 | C7 | L2 | C8 | L2 | C9 |
| L3 | C7 | L3 | C8 | L3 | C9 |
| L4 | C7 | L4 | C8 | L4 | C9 |
| L5 | C7 | L5 | C8 | L5 | C9 |
| L6 | C7 | L6 | C8 | L6 | C9 |
| L7 | C7 | L7 | C8 | L7 | C9 |
| L8 | C7 | L8 | C8 | L8 | C9 |
| L9 | C7 | L9 | C8 | L9 | C9 |
| L10 | C7 | L10 | C8 | L10 | C9 |
| L11 | C7 | L11 | C8 | L11 | C9 |
| L12 | C7 | L12 | C8 | L12 | C9 |
| L13 | C7 | L13 | C8 | L13 | C9 |
| L14 | C7 | L14 | C8 | L14 | C9 |
| L15 | C7 | L15 | C8 | L15 | C9 |
| L16 | C7 | L16 | C8 | L16 | C9 |
| L17 | C7 | L17 | C8 | L17 | C9 |
| L18 | C7 | L18 | C8 | L18 | C9 |
| L19 | C7 | L19 | C8 | L19 | C9 |
| L1 | C10 | L1 | C11 | L1 | C12 |
| L2 | C10 | L2 | C11 | L2 | C12 |
| L3 | C10 | L3 | C11 | L3 | C12 |
| L4 | C10 | L4 | C11 | L4 | C12 |
| L5 | C10 | L5 | C11 | L5 | C12 |
| L6 | C10 | L6 | C11 | L6 | C12 |
| L7 | C10 | L7 | C11 | L7 | C12 |
| L8 | C10 | L8 | C11 | L8 | C12 |
| L9 | C10 | L9 | C11 | L9 | C12 |
| L10 | C10 | L10 | C11 | L10 | C12 |
| L11 | C10 | L11 | C11 | L11 | C12 |
| L12 | C10 | L12 | C11 | L12 | C12 |
| L13 | C10 | L13 | C11 | L13 | C12 |
| L14 | C10 | L14 | C11 | L14 | C12 |
| L15 | C10 | L15 | C11 | L15 | C12 |
| L16 | C10 | L16 | C11 | L16 | C12 |
| L17 | C10 | L17 | C11 | L17 | C12 |
| L18 | C10 | L18 | C11 | L18 | C12 |
| L19 | C10 | L19 | C11 | L19 | C12 |

In certain embodiments, a hepcidin analogue comprises two or more linkers. In particular embodiments, the two or more linkers are concatamerized, i.e., bound to each other.

In related embodiments, the present invention includes polynucleotides that encode a polypeptide having a peptide sequence present in any of the hepcidin analogues described herein.

In addition, the present invention includes vectors, e.g., expression vectors, comprising a polynucleotide of the present invention.

### Methods of Treatment

In some embodiments, the present invention provides methods for treating a subject afflicted with a disease or disorder associated with dysregulated hepcidin signaling, wherein the method comprises administering to the subject a hepcidin analogue of the present invention. In some embodiments, the hepcidin analogue that is administered to the subject is present in a composition (e.g., a pharmaceutical composition). In one embodiment, a method is provided for treating a subject afflicted with a disease or disorder characterized by increased activity or expression of ferroportin, wherein the method comprises administering to the individual a hepcidin analogue or composition of the present invention in an amount sufficient to (partially or fully) bind to and agonize ferroportin in the subject. In one embodiment, a method is provided for treating a subject afflicted with a disease or disorder characterized by dysregulated iron metabolism, wherein the method comprises administering to the subject a hepcidin analogue or composition of the present invention.

In some embodiments, methods of the present invention comprise providing a hepcidin analogue or a composition of the present invention to a subject in need thereof. In particular embodiments, the subject in need thereof has been diagnosed with or has been determined to be at risk of developing a disease or disorder characterized by dysregulated iron levels (e.g., diseases or disorders of iron metabolism; diseases or disorders related to iron overload; and diseases or disorders related to abnormal hepcidin activity or expression). In particular embodiments, the subject is a mammal (e.g., a human).

In certain embodiments, the disease or disorder is a disease of iron metabolism, such as, e.g., an iron overload disease, iron deficiency disorder, disorder of iron biodistribution, or another disorder of iron metabolism and other disorder potentially related to iron metabolism, etc. In particular embodiments, the disease of iron metabolism is hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, beta thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease, transfusion-dependent anemia, hemolytic anemia, erythropoietin resistance, iron deficiency of obesity, other anemias, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer (e.g., liver cancer), hepatitis, cirrhosis of liver, pica, chronic renal failure, insulin resistance, diabetes, atherosclerosis, neurodegenerative disorders, dementia, multiple sclerosis, Parkinson's disease, Huntington's disease, or Alzheimer's disease.

In certain embodiments, the disease or disorder is related to iron overload diseases such as iron hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia.

In certain embodiments, the disease or disorder is one that is not typically identified as being iron related. For example, hepcidin is highly expressed in the murine pancreas suggesting that diabetes (Type I or Type II), insulin resistance, glucose intolerance and other disorders may be ameliorated by treating underlying iron metabolism disorders. See Ilyin, G. et al. (2003) FEBS Lett. 542 22-26, which is herein incorporated by reference. As such, peptides of the invention may be used to treat these diseases and conditions. Those skilled in the art are readily able to determine whether a given disease can be treated with a peptide according to the present invention using methods known in the art, including the assays of WO 2004092405, which is herein incorporated by reference, and assays which monitor hepcidin, hemojuvelin, or iron levels and expression, which are known in the art such as those described in U.S. Pat. No. 7,534,764, which is herein incorporated by reference.

In certain embodiments, the disease or disorder is postmenopausal osteoporosis.

In certain embodiments of the present invention, the diseases of iron metabolism are iron overload diseases, which include hereditary hemochromatosis, iron-loading anemias, alcoholic liver diseases, heart disease and/or failure, cardiomyopathy, and chronic hepatitis C.

In particular embodiments, any of these diseases, disorders, or indications are caused by or associated with a deficiency of hepcidin or iron overload.

In some embodiments, methods of the present invention comprise providing a hepcidin analogue of the present invention (i.e., a first therapeutic agent) to a subject in need thereof in combination with a second therapeutic agent. In certain embodiments, the second therapeutic agent is provided to the subject before and/or simultaneously with and/or after the pharmaceutical composition is administered to the subject. In particular embodiments, the second therapeutic agent is iron chelator. In certain embodiments, the second therapeutic agent is selected from the iron chelators Deferoxamine and Deferasirox (Exjade™). In another embodiment, the method comprises administering to the subject a third therapeutic agent.

The present invention provides compositions (for example pharmaceutical compositions) comprising one or more hepcidin analogues of the present invention and a pharmaceutically acceptable carrier, excipient or diluent. A pharmaceutically acceptable carrier, diluent or excipient refers to a non-toxic solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17th edition, Alfonso R. Gennaro (Ed.), Mark Publishing Company, Easton, PA, USA, 1985. For example, sterile saline and phosphate-buffered saline at slightly acidic or physiological pH may be used. Suitable pH-buffering agents may, e.g., be phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate (e.g. as sodium acetate), or mixtures thereof. The term further encompasses any carrier agents listed in the US Pharmacopeia for use in animals, including humans.

In certain embodiments, the compositions comprise two or more hepcidin analogues disclosed herein. In certain embodiments, the combination is selected from one of the following: (i) any two or more of the hepcidin analogue peptide monomers shown therein; (ii) any two or more of the hepcidin analogue peptide dimers disclosed herein; (iii) any one or more of the hepcidin analogue peptide monomers disclosed herein, and any one or more of the hepcidin analogue peptide dimers disclosed herein.

It is to be understood that the inclusion of a hepcidin analogue of the invention (i.e., one or more hepcidin analogue peptide monomers of the invention or one or more hepcidin analogue peptide dimers of the present invention) in a pharmaceutical composition also encompasses inclusion of a pharmaceutically acceptable salt or solvate of a hepcidin analogue of the invention. In particular embodiments, the pharmaceutical compositions further comprise one or more pharmaceutically acceptable carrier, excipient, or vehicle.

In certain embodiments, the invention provides a pharmaceutical composition comprising a hepcidin analogue, or a pharmaceutically acceptable salt or solvate thereof, for treating a variety of conditions, diseases, or disorders as disclosed herein or elsewhere (see, e.g., Methods of Treatment, herein). In particular embodiments, the invention provides a pharmaceutical composition comprising a hepcidin analogue peptide monomer, or a pharmaceutically acceptable salt or solvate thereof, for treating a variety of conditions, diseases, or disorders as disclosed herein elsewhere (see, e.g., Methods of Treatment, herein). In particular embodiments, the invention provides a pharmaceutical composition comprising a hepcidin analogue peptide dimer, or a pharmaceutically acceptable salt or solvate thereof, for treating a variety of conditions, diseases, or disorders as disclosed herein.

The hepcidin analogues of the present invention may be formulated as pharmaceutical compositions which are suited for administration with or without storage, and which typically comprise a therapeutically effective amount of at least one hepcidin analogue of the invention, together with a pharmaceutically acceptable carrier, excipient or vehicle.

In some embodiments, the hepcidin analogue pharmaceutical compositions of the invention are in unit dosage form. In such forms, the composition is divided into unit doses containing appropriate quantities of the active component or components. The unit dosage form may be presented as a packaged preparation, the package containing discrete quantities of the preparation, for example, packaged tablets, capsules or powders in vials or ampoules. The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition. Compositions may be formulated for any suitable route and means of administration, e.g., any one of the routes and means of administration disclosed herein.

In particular embodiments, the hepcidin analogue, or the pharmaceutical composition comprising a hepcidin analogue, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

In certain embodiments, the compositions are administered parenterally, subcutaneously or orally. In particular embodiments, the compositions are administered orally, intracisternally, intravaginally, intraperitoneally, intrarectally, topically (as by powders, ointments, drops, suppository, or transdermal patch, including delivery intravitreally, intranasally, and via inhalation) or buccally. The term “parenteral” as used herein refers to modes of administration which include intravenous, intramuscular, intraperitoneal, intrasternal, subcutaneous, intradermal and intra-articular injection and infusion. Accordingly, in certain embodiments, the compositions are formulated for delivery by any of these routes of administration.

In certain embodiments, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, beta-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsule matrices of the hepcidin analogue in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the hepcidin analogue can be controlled. Depot injectable formulations are also prepared by entrapping the hepcidin analogue in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Hepcidin analogues of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a hepcidin analogue of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions to be used in the invention suitable for parenteral administration may comprise sterile aqueous solutions and/or suspensions of the peptide inhibitors made isotonic with the blood of the recipient, generally using sodium chloride, glycerin, glucose, mannitol, sorbitol, and the like.

In some aspects, the invention provides a pharmaceutical composition for oral delivery. Compositions and hepcidin analogues of the instant invention may be prepared for oral administration according to any of the methods, techniques, and/or delivery vehicles described herein. Further, one having skill in the art will appreciate that the hepcidin analogues of the instant invention may be modified or integrated into a system or delivery vehicle that is not disclosed herein, yet is well known in the art and compatible for use in oral delivery of peptides.

In certain embodiments, formulations for oral administration may comprise adjuvants (e.g. resorcinols and/or nonionic surfactants such as polyoxyethylene oleyl ether and n-hexadecylpolyethylene ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the hepcidin analogue of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular embodiments, oral dosage forms or unit doses compatible for use with the hepcidin analogues of the present invention may include a mixture of hepcidin analogue and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of hepcidin analogue, wherein the dosage form comprises at least one of a pill, a tablet, a capsule, a gel, a paste, a drink, a syrup, ointment, and suppository. In some instances, an oral dosage form is provided that is designed and configured to achieve delayed release of the hepcidin analogue in the subject's small intestine and/or colon.

In one embodiment, an oral pharmaceutical composition comprising a hepcidin analogue of the present invention comprises an enteric coating that is designed to delay release of the hepcidin analogue in the small intestine. In at least some embodiments, a pharmaceutical composition is provided which comprises a hepcidin analogue of the present invention and a protease inhibitor, such as aprotinin, in a delayed release pharmaceutical formulation. In some instances, pharmaceutical compositions of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a hepcidin analogue of the present invention is provided in an enteric coating, the enteric coating being designed to protect and release the pharmaceutical composition in a controlled manner within the subject's lower gastrointestinal system, and to avoid systemic side effects. In addition to enteric coatings, the hepcidin analogues of the instant invention may be encapsulated, coated, engaged or otherwise associated within any compatible oral drug delivery system or component. For example, in some embodiments a hepcidin analogue of the present invention is provided in a lipid carrier system comprising at least one of polymeric hydrogels, nanoparticles, microspheres, micelles, and other lipid systems.

To overcome peptide degradation in the small intestine, some embodiments of the present invention comprise a hydrogel polymer carrier system in which a hepcidin analogue of the present invention is contained, whereby the hydrogel polymer protects the hepcidin analogue from proteolysis in the small intestine and/or colon. The hepcidin analogues of the present invention may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more hepcidin analogue of the present invention to provide a pharmaceutical agent for oral delivery. In some embodiments, a hepcidin analogue of the instant invention is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a hepcidin analogue disclosed herein, wherein the surface of the hepcidin analogue surface is modified to comprise mucoadhesive properties through hydrogen bonds, polymers with linked mucins or/and hydrophobic interactions. These modified peptide molecules may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the hepcidin analogue.

Other embodiments comprise a method for oral delivery of a hepcidin analogue of the present invention, wherein the hepcidin analogue is provided to a subject in combination with permeation enhancers that promote the transport of the peptides across the intestinal mucosa by increasing paracellular or transcellular permeation. For example, in one embodiment, a permeation enhancer is combined with a hepcidin analogue, wherein the permeation enhancer comprises at least one of a long-chain fatty acid, a bile salt, an amphiphilic surfactant, and a chelating agent. In one embodiment, a permeation enhancer comprising sodium N-[hydroxybenzoyl)amino] caprylate is used to form a weak noncovalent association with the hepcidin analogue of the instant invention, wherein the permeation enhancer favors membrane transport and further dissociation once reaching the blood circulation. In another embodiment, a hepcidin analogue of the present invention is conjugated to oligoarginine, thereby increasing cellular penetration of the peptide into various cell types. Further, in at least one embodiment a noncovalent bond is provided between a peptide inhibitor of the present invention and a permeation enhancer selected from the group consisting of a cyclodextrin (CD) and a dendrimers, wherein the permeation enhancer reduces peptide aggregation and increasing stability and solubility for the hepcidin analogue molecule.

Other embodiments of the invention provide a method for treating a subject with a hepcidin analogue of the present invention having an increased half-life. In one aspect, the present invention provides a hepcidin analogue having a half-life of at least several hours to one day in vitro or in vivo (e.g., when administered to a human subject) sufficient for daily (q.d.) or twice daily (b.i.d.) dosing of a therapeutically effective amount. In another embodiment, the hepcidin analogue has a half-life of three days or longer sufficient for weekly (q.w.) dosing of a therapeutically effective amount. Further, in another embodiment, the hepcidin analogue has a half-life of eight days or longer sufficient for bi-weekly (b.i.w.) or monthly dosing of a therapeutically effective amount. In another embodiment, the hepcidin analogue is derivatized or modified such that is has a longer half-life as compared to the underivatized or unmodified hepcidin analogue. In another embodiment, the hepcidin analogue contains one or more chemical modifications to increase serum half-life.

When used in at least one of the treatments or delivery systems described herein, a hepcidin analogue of the present invention may be employed in pure form or, where such forms exist, in pharmaceutically acceptable salt form.

### Dosages

The total daily usage of the hepcidin analogues and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific hepcidin analogue employed; e) the duration of the treatment; f) drugs used in combination or coincidental with the specific hepcidin analogue employed, and like factors well known in the medical arts.

In particular embodiments, the total daily dose of the hepcidin analogues of the invention to be administered to a human or other mammal host in single or divided doses may be in amounts, for example, from 0.0001 to 300 mg/kg body weight daily or 1 to 300 mg/kg body weight daily. In certain embodiments, a dosage of a hepcidin analogue of the present invention is in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses. In particular embodiments, a total dosage is about 1 mg, about 2 mg, about 3 mg, about 4 mg, about 5 mg, about 6 mg, about 7 mg, about 8 mg, about 9 mg, or about 10 mg about once or twice weekly, e.g., for a human patient. In particular embodiments, the total dosage is in the range of about 1 mg to about 5 mg, or about 1 mg to about 3 mg, or about 2 mg to about 3 mg per human patient, e.g., about once weekly.

In various embodiments, a hepcidin analogue of the invention may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like.

Such regular hepcidin analogue administration regimens of the invention may, in certain circumstances such as, e.g., during chronic long-term administration, be advantageously interrupted for a period of time so that the medicated subject reduces the level of or stops taking the medication, often referred to as taking a “drug holiday.” Drug holidays are useful for, e.g., maintaining or regaining sensitivity to a drug especially during long-term chronic treatment, or to reduce unwanted side-effects of long-term chronic treatment of the subject with the drug. The timing of a drug holiday depends on the timing of the regular dosing regimen and the purpose for taking the drug holiday (e.g., to regain drug sensitivity and/or to reduce unwanted side effects of continuous, long-term administration). In some embodiments, the drug holiday may be a reduction in the dosage of the drug (e.g. to below the therapeutically effective amount for a certain interval of time). In other embodiments, administration of the drug is stopped for a certain interval of time before administration is started again using the same or a different dosing regimen (e.g. at a lower or higher dose and/or frequency of administration). A drug holiday of the invention may thus be selected from a wide range of time-periods and dosage regimens. An exemplary drug holiday is two or more days, one or more weeks, or one or more months, up to about 24 months of drug holiday. So, for example, a regular daily dosing regimen with a peptide, a peptide analogue, or a dimer of the invention may, for example, be interrupted by a drug holiday of a week, or two weeks, or four weeks, after which time the preceding, regular dosage regimen (e.g. a daily or a weekly dosing regimen) is resumed. A variety of other drug holiday regimens are envisioned to be useful for administering the hepcidin analogues of the invention.

Thus, the hepcidin analogues may be delivered via an administration regime which comprises two or more administration phases separated by respective drug holiday phases.

During each administration phase, the hepcidin analogue is administered to the recipient subject in a therapeutically effective amount according to a pre-determined administration pattern. The administration pattern may comprise continuous administration of the drug to the recipient subject over the duration of the administration phase. Alternatively, the administration pattern may comprise administration of a plurality of doses of the hepcidin analogue to the recipient subject, wherein said doses are spaced by dosing intervals.

A dosing pattern may comprise at least two doses per administration phase, at least five doses per administration phase, at least 10 doses per administration phase, at least 20 doses per administration phase, at least 30 doses per administration phase, or more.

Said dosing intervals may be regular dosing intervals, which may be as set out above, including once daily, twice daily, once every two, three, four, five or six days, once or twice weekly, once or twice monthly, or a regular and even less frequent dosing interval, depending on the particular dosage formulation, bioavailability, and pharmacokinetic profile of the hepcidin analogue of the present invention.

An administration phase may have a duration of at least two days, at least a week, at least 2 weeks, at least 4 weeks, at least a month, at least 2 months, at least 3 months, at least 6 months, or more.

Where an administration pattern comprises a plurality of doses, the duration of the following drug holiday phase is longer than the dosing interval used in that administration pattern. Where the dosing interval is irregular, the duration of the drug holiday phase may be greater than the mean interval between doses over the course of the administration phase. Alternatively the duration of the drug holiday may be longer than the longest interval between consecutive doses during the administration phase.

The duration of the drug holiday phase may be at least twice that of the relevant dosing interval (or mean thereof), at least 3 times, at least 4 times, at least 5 times, at least 10 times, or at least 20 times that of the relevant dosing interval or mean thereof.

Within these constraints, a drug holiday phase may have a duration of at least two days, at least a week, at least 2 weeks, at least 4 weeks, at least a month, at least 2 months, at least 3 months, at least 6 months, or more, depending on the administration pattern during the previous administration phase.

An administration regime comprises at least 2 administration phases. Consecutive administration phases are separated by respective drug holiday phases. Thus the administration regime may comprise at least 3, at least 4, at least 5, at least 10, at least 15, at least 20, at least 25, or at least 30 administration phases, or more, each separated by respective drug holiday phases.

Consecutive administration phases may utilise the same administration pattern, although this may not always be desirable or necessary. However, if other drugs or active agents are administered in combination with a hepcidin analogue of the invention, then typically the same combination of drugs or active agents is given in consecutive administration phases. In certain embodiments, the recipient subject is human.

In some embodiments, the present invention provides compositions and medicaments comprising at least one hepcidin analogue as disclosed herein. In some embodiments, the present invention provides a method of manufacturing medicaments comprising at least one hepcidin analogue as disclosed herein for the treatment of diseases of iron metabolism, such as iron overload diseases. In some embodiments, the present invention provides a method of manufacturing medicaments comprising at least one hepcidin analogue as disclosed herein for the treatment of diabetes (Type I or Type II), insulin resistance, or glucose intolerance. Also provided are methods of treating a disease of iron metabolism in a subject, such as a mammalian subject, and preferably a human subject, comprising administering at least one hepcidin analogue, or composition as disclosed herein to the subject. In some embodiments, the hepcidin analogue or the composition is administered in a therapeutically effective amount. Also provided are methods of treating diabetes (Type I or Type II), insulin resistance, or glucose intolerance in a subject, such as a mammalian subject, and preferably a human subject, comprising administering at least one hepcidin analogue or composition as disclosed herein to the subject. In some embodiments, the hepcidin analogue or composition is administered in a therapeutically effective amount.

In some embodiments, the invention provides a process for manufacturing a hepcidin analogue or a hepcidin analogue composition (e.g., a pharmaceutical composition), as disclosed herein.

In some embodiments, the invention provides a device comprising at least one hepcidin analogue of the present invention, or pharmaceutically acceptable salt or solvate thereof for delivery of the hepcidin analogue to a subject.

In some embodiments, the present invention provides methods of binding a ferroportin or inducing ferroportin internalization and degradation which comprises contacting the ferroportin with at least one hepcidin analogue, or hepcidin analogue composition as disclosed herein.

In some embodiments, the present invention provides kits comprising at least one hepcidin analogue, or hepcidin analogue composition (e.g., pharmaceutical composition) as disclosed herein packaged together with a reagent, a device, instructional material, or a combination thereof.

In some embodiments, the present invention provides a method of administering a hepcidin analogue or hepcidin analogue composition (e.g., pharmaceutical composition) of the present invention to a subject via implant or osmotic pump, by cartridge or micro pump, or by other means appreciated by the skilled artisan, as well-known in the art.

In some embodiments, the present invention provides complexes which comprise at least one hepcidin analogue as disclosed herein bound to a ferroportin, preferably a human ferroportin, or an antibody, such as an antibody which specifically binds a hepcidin analogue as disclosed herein, Hep25, or a combination thereof.

In some embodiments, the hepcidin analogue of the present invention has a measurement (e.g., an EC₅₀) of less than 500 nM within the FPN internalization assay. As a skilled person will realize, the function of the hepcidin analogue is dependent on the tertiary structure of the hepcidin analogue and the binding surface presented. It is therefore possible to make minor changes to the sequence encoding the hepcidin analogue that do not affect the fold or are not on the binding surface and maintain function. In other embodiments, the present invention provides a hepcidin analogue having 85% or higher (e.g., 85%, 90%, 91%, 92%, 93%, 94%, 95%, 96%, 97%, 98%, 99%, or 99.5%) identity or homology to an amino acid sequence of any hepcidin analogue described herein that exhibits an activity (e.g., hepcidin activity), or lessens a symptom of a disease or indication for which hepcidin is involved.

In other embodiments, the present invention provides a hepcidin analogue having 85% or higher (e.g., 85%, 90%, 91%, 92%, 93%, 94%, 95%, 96%, 97%, 98%, 99%, or 99.5%) identity or homology to an amino acid sequence of any hepcidin analogue presented herein, or a peptide according to any one of the formulae or hepcidin analogues described herein.

In some embodiments, a hepcidin analogue of the present invention may comprise functional fragments or variants thereof that have at most 10, 9, 8, 7, 6, 5, 4, 3, 2, or 1 amino acid substitutions compared to one or more of the specific peptide analogue sequences recited herein.

In addition to the methods described in the Examples herein, the hepcidin analogues of the present invention may be produced using methods known in the art including chemical synthesis, biosynthesis or in vitro synthesis using recombinant DNA methods, and solid phase synthesis. See e.g. Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. The hepcidin analogues of the present invention may be purified using protein purification techniques known in the art such as reverse phase high-performance liquid chromatography (HPLC), ion-exchange or immunoaffinity chromatography, filtration or size exclusion, or electrophoresis. See Olsnes, S. and A. Pihl (1973) Biochem. 12(16):3121-3126; and Scopes (1982) Protein Purification, Springer-Verlag, NY, which are herein incorporated by reference. Alternatively, the hepcidin analogues of the present invention may be made by recombinant DNA techniques known in the art. Thus, polynucleotides that encode the polypeptides of the present invention are contemplated herein. In certain preferred embodiments, the polynucleotides are isolated. As used herein “isolated polynucleotides” refers to polynucleotides that are in an environment different from that in which the polynucleotide naturally occurs.

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed in any way as limiting the scope of the present invention.

### ABBREVIATIONS

DCM: dichloromethane
DMF: N,N-dimethylformamide
NMP: N-methylpyrolidone
HBTU: O-(Benzotriazol-1-yl)-N,N,N′,N′-tetramethyluronium hexafluorophosphate
HATU: 2-(7-aza-1H-benzotriazole-1-yl)-1,1,3,3-tetramethyluronium hexafluorophosphate
DCC: Dicyclohexylcarbodiimide
NHS: N-hydoxysuccinimide
DIEA: diisopropylethylamine
EtOH: ethanol
Et2O: diethyl ether
Hy: hydrogen
TFA: trifluoroacetic acid
TIS: triisopropylsilane
ACN: acetonitrile
HPLC: high performance liquid chromatography
ESI-MS: electron spray ionization mass spectrometry
PBS: phosphate-buffered saline
Boc: t-butoxycarbonyl
Fmoc: Fluorenylmethyloxycarbonyl
Acm: acetamidomethyl
IVA: Isovaleric acid (or Isovaleryl)

K( ): In the peptide sequences provided herein, wherein a compound or chemical group is presented in parentheses directly after a Lysine residue, it is to be understood that the compound or chemical group in the parentheses is a side chain conjugated to the Lysine residue. So, e.g., but not to be limited in any way, K-[(1PEG8)]- indicates that a PEG8 moiety is conjugated to a side chain of this Lysine.

Palm: Indicates conjugation of a palmitic acid (palmitoyl).

As used herein “C( )” refers to a cysteine residue involved in a particular disulfide bridge. For example, in Hepcidin, there are four disulfide bridges: the first between the two C(1) residues; the second between the two C(2) residues; the third between the two C(3) residues; and the fourth between the two C(4) residues. Accordingly, in some embodiments, the sequence for Hepcidin is written as follows: Hy-DTHFPIC(1)IFC(2)C(3)GC(2)C(4)HRSKC(3)GMC(4)C(1)KT-OH (SEQ ID NO:156); and the sequence for other peptides may also optionally be written in the same manner.

### Example 1

### Synthesis of Peptide Analogues

Unless otherwise specified, reagents and solvents employed in the following were available commercially in standard laboratory reagent or analytical grade, and were used without further purification.

### Procedure for Solid-Phase Synthesis of Peptides

### Method A

Peptide analogues of the invention were chemically synthesized using optimized 9-fluorenylmethoxy carbonyl (Fmoc) solid phase peptide synthesis protocols. For C-terminal amides, rink-amide resin was used, although wang and trityl resins were also used to produce C-terminal acids. The side chain protecting groups were as follows: Glu, Thr and Tyr: O-tButyl; Trp and Lys: t-Boc (t-butyloxycarbonyl); Arg: N-gamma-2,2,4,6,7-pentamethyldihydrobenzofuran-5-sulfonyl; His, Gln, Asn, Cys: Trityl. For selective disulfide bridge formation, Acm (acetamidomethyl) was also used as a Cys protecting group. For coupling, a four to ten-fold excess of a solution containing Fmoc amino acid, HBTU and DIEA (1:1:1.1) in DMF was added to swelled resin [HBTU: 0-(Benzotriazol-1-yl)-N,N,N′,N′-tetramethyluronium hexafluorophosphate; DIEA: diisopropylethylamine; DMF: dimethylformamide]. HATU (O-(7-azabenzotriazol-1-yl)-1,1,3,3,-tetramethyluronium hexafluorophosphate) was used instead of HBTU to improve coupling efficiency in difficult regions. Fmoc protecting group removal was achieved by treatment with a DMF, piperidine (2:1) solution.

### Method B

Alternatively, peptides were synthesized utilizing the CEM liberty Blue Microwave assisted peptide synthesizer. Using the Liberty Blue, FMOC deprotection was carried out by addition of 20% 4-methylpiperdine in DMF with 0.1M Oxyma in DMF and then heating to 90° C. using microwave irradiation for 4 min. After DMF washes the FMOC-amino acids were coupled by addition of 0.2M amino acid (4-6 eq), 0.5M DIC (4-6 eq) and 1M Oxyma (with 0.1M DIEA) 4-6 eq (all in DMF). The coupling solution is heated using microwave radiation to 90° C. for 4 min. A second coupling is employed when coupling Arg or other sterically hindered amino acids. When coupling with histidine, the reaction is heated to 50° C. for 10 min. The cycles are repeated until the full length peptide is obtained.

### Procedure for Cleavage of Peptides Off Resin

Side chain deprotection and cleavage of the peptide analogues of the invention (e.g., Compound No. 2) was achieved by stirring dry resin in a solution containing trifluoroacetic acid, water, ethanedithiol and tri-isopropylsilane (90:5:2.5:2.5) for 2 to 4 hours. Following TFA removal, peptide was precipitated using ice-cold diethyl ether. The solution was centrifuged and the ether was decanted, followed by a second diethyl ether wash. The peptide was dissolved in an acetonitrile, water solution (1:1) containing 0.1% TFA (trifluoroacetic acid) and the resulting solution was filtered. The linear peptide quality was assessed using electrospray ionization mass spectrometry (ESI-MS).

### Procedure for Purification of Peptides

Purification of the peptides of the invention (e.g., Compound No. 2) was achieved using reverse-phase high performance liquid chromatography (RP-HPLC). Analysis was performed using a C18 column (3 μm, 50×2 mm) with a flow rate of 1 mL/min. Purification of the linear peptides was achieved using preparative RP-HPLC with a C18 column (5 μm, 250×21.2 mm) with a flow rate of 20 mL/min. Separation was achieved using linear gradients of buffer B in A (Buffer A: Aqueous 0.05% TFA; Buffer B: 0.043% TFA, 90% acetonitrile in water).

### Procedure for Oxidation of Peptides

Method A (Single disulfide oxidation). Oxidation of the unprotected peptides of the invention was achieved by adding drop-wise iodine in MeOH (1 mg per 1 mL) to the peptide in a solution (ACN: H₂O, 7: 3, 0.5% TFA). After stirring for 2 min, ascorbic acid portion wise was added until the solution was clear and the sample was immediately loaded onto the HPLC for purification.

Method B (Selective oxidation of two disulfides). When more than one disulfide was present, selective oxidation was often performed. Oxidation of the free cysteines was achieved at pH 7.6 NH₄CO₃ solution at 1 mg/10 mL of peptide. After 24 h stirring and prior to purification the solution was acidified to pH 3 with TFA followed by lyophilization. The resulting single oxidized peptides (with ACM protected cysteines) were then oxidized/selective deprotection using iodine solution. The peptide (1 mg per 2 mL) was dissolved in MeOH/H₂O, 80:20 iodine dissolved in the reaction solvent was added to the reaction (final concentration: 5 mg/mL) at room temperature. The solution was stirred for 7 minutes before ascorbic acid was added portion wise until the solution is clear. The solution was then loaded directly onto the HPLC.

Method C (Native oxidation). When more than one disulfide was present and when not performing selective oxidations, native oxidation was performed. Native oxidation was achieved with 100 mM NH4CO3 (pH7.4) solution in the presence of oxidized and reduced glutathione (peptide/GSH/GSSG, 1:100:10 molar ratio) of (peptide: GSSG: GSH, 1:10, 100). After 24 h stirring and prior to RP-HPLC purification the solution was acidified to pH 3 with TFA followed by lyophilization.

### Procedure of Cysteine Oxidation to Produce Dimers.

Oxidation of the unprotected peptides of the invention was achieved by adding drop-wise iodine in MeOH (1 mg per 1 mL) to the peptide in a solution (ACN: H₂O, 7: 3, 0.5% TFA). After stirring for 2 min, ascorbic acid portion wise was added until the solution was clear and the sample was immediately loaded onto the HPLC for purification.

### Procedure for Dimerization.

Glyoxylic acid (DIG), IDA, or Fmoc-β-Ala-IDA was pre-activated as the N-hydoxysuccinimide ester by treating 1 equivalent (abbreviated “eq”) of the acid with 2.2 eq of both N-hydoxysuccinimide (NHS) and dicyclohexyl carbodiimide (DCC) in NMP (N-methyl pyrolidone) at a 0.1 M final concentration. For the PEG13 and PEG25 linkers, these chemical entities were purchased pre-formed as the activated succinimide ester. The activated ester ˜ 0.4 eq was added slowly to the peptide in NMP (1 mg/mL) portionwise. The solution was left stirring for 10 min before 2-3 additional aliquots of the linker -0.05 eq were slowly added. The solution was left stirring for a further 3 h before the solvent was removed under vaccuo and the residue was purified by reverse phase HPLC. An additional step of stirring the peptide in 20% piperidine in DMF (2×10 min) before an additional reverse phase HPLC purification was performed.

One of skill in the art will appreciate that standard methods of peptide synthesis may be used to generate the compounds of the invention.

### Linker Activation and Dimerization

Peptide monomer subunits were linked to form hepcidin analogue peptide dimers as described below.

Small Scale DIG Linker Activation Procedure: 5 mL of NMP was added to a glass vial containing IDA diacid (304.2 mg, 1 mmol), N-hydroxysuccinimide (NHS, 253.2 mg, 2.2 eq. 2.2 mmol) and a stirring bar. The mixture was stirred at room temperature to completely dissolve the solid starting materials. N, N′-Dicyclohexylcarbodiimide (DCC, 453.9 mg, 2.2 eq., 2.2 mmol) was then added to the mixture. Precipitation appeared within 10 min and the reaction mixture was further stirred at room temperature overnight. The reaction mixture was then filtered to remove the precipitated dicyclohexylurea (DCU). The activated linker was kept in a closed vial prior to use for dimerization. The nominal concentration of the activated linker was approximately 0.20 M.

For dimerization using PEG linkers, there was no pre-activation step involved. Commercially available pre-activated bi-functional PEG linkers were used.

Dimerization Procedure: 2 mL of anhydrous DMF was added to a vial containing peptide monomer (0.1 mmol). The pH of the peptide was the adjusted to 8-9 with DIEA. Activated linker (IDA or PEG13, PEG 25) (0.48eq relative to monomer, 0.048 mmol) was then added to the monomer solution. The reaction mixture was stirred at room temperature for one hour. Completion of the dimerization reaction was monitored using analytical HPLC. The time for completion of dimerization reaction varied depending upon the linker. After completion of reaction, the peptide was precipitated in cold ether and centrifuged. The supernatant ether layer was discarded. The precipitation step was repeated twice. The crude dimer was then purified using reverse phase HPLC (Luna C18 support, 10u, 100A, Mobile phase A: water containing 0.1% TFA, mobile phase B: Acetonitrile (ACN) containing 0.1% TFA, gradient of 15% B and change to 45% B over 60 min, flow rate 15 ml/min). Fractions containing pure product were then freeze-dried on a lyophilizer.

### Conjugation of Half-Life Extension Moieties

Conjugation of peptides were performed on resin. Lys(ivDde) was used as the key amino acid. After assembly of the peptide on resin, selective deprotection of the ivDde group occurred using 3×5 min 2% hydrazine in DMF for 5 min. Activation and acylation of the linker using HBTU, DIEA 1-2 equivalents for 3 h, and Fmoc removal followed by a second acylation with the lipidic acid gave the conjugated peptide.

### Example 2

### Activity of Peptide Analogues

Peptide analogues were tested in vitro for induction of internalization of the human ferroportin protein. Following internalization, the ferroporin protein is degraded. The assay used (FPN activity assay) measures a decrease in fluorescence of the receptor.

The cDNA encoding the human ferroportin (SLC40A1) was cloned from a cDNA clone from Origene (NM_014585). The DNA encoding the ferroportin was amplified by PCR using primers also encoding terminal restriction sites for subcloning, but without the termination codon. The ferroportin receptor was subcloned into a mammalian GFP expression vector containing a neomycin (G418) resistance marker in such that the reading frame of the ferroportin was fused in frame with the GFP protein. The fidelity of the DNA encoding the protein was confirmed by DNA sequencing. HEK293 cells were used for transfection of the ferroportin-GFP receptor expression plasmid. The cells were grown according to standard protocol in growth medium and transfected with the plasmids using Lipofectamine (manufacturer's protocol, Invitrogen). The cells stably expressing ferroportin-GFP were selected using G418 in the growth medium (in that only cells that have taken up and incorporated the cDNA expression plasmid survive) and sorted several times on a Cytomation MoFlo™ cell sorter to obtain the GFP-positive cells (488 nm/530 nm). The cells were propagated and frozen in aliquots.

To determine activity of the hepcidin analogues (compounds) on the human ferroportin, the cells were incubated in 96 well plates in standard media, without phenol red. Compound was added to desired final concentration for at least 18 hours in the incubator. Following incubation, the remaining GFP-fluorescence was determined either by whole cell GFP fluorescence (Envision plate reader, 485/535 filter pair), or by Beckman Coulter Quanta™ flow cytometer (express as Geometric mean of fluorescence intensity at 485 nm/525 nm). Compound was added to desired final concentration for at least 18 hours but no more than 24 hours in the incubator.

In certain experiments, reference compounds included native Hepcidin, Mini-Hepcidin, and R¹-Mini-Hepcidin, which is an analog of mini-hepcidin. The “RI” in RI-Mini-Hepcidin refers to Retro Inverse. A retro inverse peptide is a peptide with a reversed sequence in all D amino acids. An example is that Hy-Glu-Thr-His-NH₂ becomes Hy-DHis-DThr-DGlu-NH₂. The EC₅₀ of these reference compounds for ferroportin internalization/degradation was determined according to the FPN activity assay described above. These peptides served as control standards.

TABLE 7
Reference compounds

| | | Potency |
|---|---|---|
| Name | Sequence | EC₅₀ (nM) |
| Hepcidin | Hy-DTHFPIC(1)IFC(2)C(3)GC(2)C(4)HRSKC(3)GMC(4)C(1)KT- | 34 |
| | OH (SEQ ID NO: 256) | |
| Mini- | Hy-DTHFPICIF-NH₂ (SEQ ID NO: 257) | 712 |
| Hepcidin | | |
| 1-9 | | |
| RI-Mini | Hy-DPhe-DIle-DCys-DIle-DPro-DPhe-DHis-DThr-DAsp-NH₂ | >10 μM |
| Hepcidin | | |
| Ref. | Isovaleric acid-DTHFPCIKF-Lys[2PEG11-Palm]-PRSKGCK-NH₂ | 30 |
| Compd 1 | | |
| Ref. | Isovaleric acid-DTHFPCIKF-Lys[2PEG11-Palm]-PRSK-[SAR]-CK- | 13 |
| Compd. 2 | NH₂ | |

The potency EC₅₀ values nM determined or various peptide analogues of the present invention are provided in Table 3. These values were determined as described herein.

### Example 3

### In Vivo Validation of Peptide Analogues

Hepcidin analogues of the present invention were tested for in vivo activity, to determine their ability to decrease free Fe2+60 in serum.

A hepcidin analogue or vehicle control were administered to mice (n=3/group) at 1000 nmol/kg either intravenously or subcutaneously. Serum samples were taken from groups of mice administered with the hepcidin analog at 30 min, 1 h, 2 h, 4 h, 10 h, 24 h, 30 h, 36 h, and 48 h post-administration. Iron content in plasma/serum was measured using a colorimetric assay on the Cobas c 111 according to instructions from the manufacturer of the assay (assay: IRON2: ACN 661).

In another experiment, various hepcidin analogues or vehicle control were administered to mice (n=3/group) at 1000 nmol/kg subcutaneously. Serum samples were taken from groups of mice administered with vehicle or hepcidin analog at 30 h and 36 h post-administration. Iron content in plasma/serum was measured using a colorimetric assay on the Cobas c 111 according to instructions from the manufacturer of the assay (assay: IRON2: ACN 661).

These studies demonstrate that hepcidin analogues of the present invention reduce serum iron levels for at least 30 hours, thus demonstrating their increased serum stability.

### Example 4

### In Vitro Validation of Peptide Analogues

Based in part on the structure activity relationships (SAR) determined from the results of the experiments described herein, a variety of Hepcidin-like peptides of the present invention were synthesized using the method described in Example 1, and in vitro activity was tested as described in Example 2. Reference compounds included native Hepcidin, Mini-Hepcidin, R1-Mini-Hepcidin, Reference Compound 1 and Reference Compound 2. EC₅₀ values of the peptides are shown in summary Table 3.

### Example 5

### Plasma Stability

Plasma stability experiments were undertaken to complement the in vivo results and assist in the design of potent, stable Ferroportin agonists. In order to predict the stability in rat and mouse plasma, ex vivo stability studies were initially performed in these matrices.

Peptides of interest (20 μM) were incubated with pre-warmed plasma (BioreclamationIVT) at 37° C. Aliquots were taken at various time points up to 24 hours (e.g. 0, 0.25, 1, 3, 6 and 24 hr), and immediately quenched with 4 volumes of organic solvent (acetonitrile/methanol (1:1) and 0.1% formic acid, containing 1 μM internal standard). Quenched samples were stored at 4° C. until the end of the experiment and centrifuged at 17,000 g for 15 minutes. The supernatant were diluted 1:1 with deionized water and analyzed using LC-MS. Percentage remaining at each time point was calculated based on the peak area ratio (analyte over internal standard) relative to the initial level at time zero. Half-lives were calculated by fitting to a first-order exponential decay equation using GraphPad.

### Example 6

### Reduction of Serum Iron in Mice

Hepcidin mimetic compounds, designed for oral stability, were tested for systemic absorption by PO dosing in a wild type mouse model C57BL/6. The animals were acclimatized in normal rodent diet for 4-5 days prior to study start and fasted overnight prior to study start. Groups of 4 animals each received either Vehicle or the Compounds. The compounds were formulated in Saline at a concentration of 5 mg/mL. The mice received dosing solution via oral gavage at volume of 200 μl per animal of body weight 20 g. Each group received 1 dose of compounds at 50 mg/kg/dose. The group marked for vehicle received only the formulation. Blood was drawn at 4 hours post-dose and serum was prepared for PK and PD measurements. The compound concentration was measured by mass spectrometry method and iron concentration in the samples was measured using the colorimetric method on Roche cobas c system.

### Example 7

### Reduction of Serum Iron in Mice

In another experiment, a new set of compounds were tested for systemic absorption by PO dosing in a wild type mouse model C57BL/6. The animals were acclimatized in normal rodent diet for 4-5 days prior to study start. Over the night prior to the first dose, the mice were switched to a low iron diet (with 2 ppm iron) and this diet was maintained during the rest of the study. Groups of 5 animals each received either Vehicle or the Compounds. The concentration of compounds was at 30 mg/mL, formulated in 0.7% NaCl+10 mM NaAcetate buffer. Food was withdrawn around 2 hours prior to each dose to ensure that the stomach was clear of any food particles prior to PO dosing. The mice received dosing solution via oral gavage at volume of 200 μl per animal of body weight 20 g. Each group received 2 doses of compound at 300 mg/kg/dose, on successive days. The group marked for vehicle received only the formulation. Blood was drawn at 4.5 hours post-last-dose and serum was prepared for PD measurements. Serum iron concentration was measured using the colorimetric method on Roche cobas c system.

### Example 8

### Pharmacodynamic Effects for the Serum Iron Reducing Abilities of a Representative Compound in Mice

In a second in vivo study, a representative compound of the present invention was tested for pharmacodynamic effect with a single dose of 300 mg/kg/dose vs. 2 doses of 300 mg/kg over two days QD (once per day). C57BL/6 mice were acclimatized in normal rodent diet for 4-5 days prior to study start. Over the night prior to the first dose, the mice were switched to a low iron diet (with 2 ppm iron) and this diet was maintained during the rest of the study. Groups of 5 animals each received either Vehicle or the Compound. The compound was formulated in 0.7% NaCl+10 mM NaAcetate buffer at 30 mg/mL concentration. Food was withdrawn around 2 hours prior to each dose to ensure that the stomach was clear of any food particles prior to PO dosing. The mice received dosing solution via oral gavage at volume of 200 μl per animal of body weight 20 g.

### Example 9

### PK/PD Effects of Oral Dosing of a Representative Compound in Mice

In another in vivo study with healthy Wild Type mouse model C57/BL6, a representative compound of the present invention was tested for PK and PD effect with multiple dosing over three days. The mice were maintained under normal rodent feed during the acclimatization and switched to iron-deficient diet (with ˜2 ppm iron) one night prior to the first dose. Groups of 5 mice each received a total of 6 doses of either vehicle or the representative compound at different dose strengths, in a BID format over three days. Mice were dosed via. oral gavage with the representative compound formulated in 0.7% saline and 10 mM Sodium Acetate. The different groups received either vehicle, 150 mg/kg/dose BID, 75 mg/kg/dose BID, 37.5 mg/kg/dose BID, or 18.75 mg/kg/dose BID. An additional group received 100 mg/kg/dose BID in addition to a total of 100 mg/kg/day of compound in drinking water (DW), thereby receiving a total dose of 300 g/kg/day. At 3 hours post-last-dose the vehicle group marked for iron-challenge and all the compound dosed groups received iron solution via. oral gavage at 4 mg/kg iron per animal. Blood was collected at 90 min post-iron-challenge to prepare serum for PK and PD measurements. The compound concentration was measured by mass spectrometry method and iron concentration in the samples was measured using the colorimetric method on Roche cobas c system.

### Example 10

### Reduction of Serum Iron in Mice

In a separate triage, a new set of compounds were tested for their pharmacodynamic effect when dosed orally in the wild type mouse model C57BL/6. The animals were acclimatized in normal rodent diet for 4-5 days prior to study start. The group of 5 animals designated to receive two doses of a representative compound received an iron-deficient diet (with 2-ppm iron) on the night prior to the first dose and all the other groups designated for single dose of different compounds were treated with iron-deficient diet for two nights prior to the compound dosing. The concentration of compounds in the dosing solution was at 30 mg/mL, formulated in 0.7% NaCl+10 mM NaAcetate buffer. Food was withdrawn around 2 hours prior to any dosing to ensure that the stomach was clear of any food particles prior to PO dosing. The mice received dosing solution via oral gavage at volume of 200 μl per animal of body weight 20 g. The group marked for vehicle received only the formulation. Blood was drawn at 4.5 hours post-last-dose and serum was prepared for PD measurements. Serum iron concentration was measured using the colorimetric method on Roche cobas c system.

### Example 11

### Stability in Simulated Gastric Fluid

Blank SGF was prepared by adding 2 g sodium chloride, 7 mL hydrochloric acid (37%) in a final volume of 1 L water, and adjusted pH to 1.2.

SGF was prepared by dissolving 320 mg Pepsin (Sigma®, P6887, from Porcine Stomach Mucosa) in 100 mL Blank SGF and stirred at room temperature for 30 minutes. The solution was filtered through 0.45 μm membrane and aliquot and stored at −20° C.

Experimental compounds of interest (at a concentration of 20 μM) were incubated with pre-warmed SGF at 37° C. Aliquots were taken at various time points up to 24 hours (e.g., 0, 0.25, 1, 3, 6 and 24 hr), and immediately quenched with 4 volumes of organic solvent (acetonitrile/methanol (1:1) and 0.1% formic acid, containing 1 μM internal standard). Quenched samples were stored at 4° C. until the end of the experiment and centrifuged at 4,000 rpm for 10 minutes. The supernatant were diluted 1:1 with deionized water and analyzed using LC-MS. Percentage remaining at each time point was calculated based on the peak area ratio (analyte over internal standard) relative to the initial level at time zero. Half-lives were calculated by fitting to a first-order exponential decay equation using GraphPad.

### Example 12

### Stability in Simulated Intestinal Fluids

Blank FaSSIF was prepared by dissolving 0.348 g NaOH, 3.954 g sodium phosphate monobasic monohydrate and 6.186 g NaCl in a final volume of 1 liter water (pH adjusted to 6.5).

FaSSIF was prepared by dissolving 1.2 g porcine pancreatin (Chem-supply, PL378) in 100 mL Blank FaSSIF and stirred at room temperature for 30 minutes. The solution was filtered through 0.45 μm membrane and aliquot and stored at −20° C.

Experimental compounds of interest (20 μM) were incubated with pre-warmed FaSSIF (1% pancreatin in final incubation mixture) at 37° C. Aliquots were taken at various time points up to 24 hours (e.g. 0, 0.25, 1, 3, 6 and 24 hr), and immediately quenched with 4 volumes of organic solvent (acetonitrile/methanol (1:1) and 0.1% formic acid, containing 1 μM internal standard). Quenched samples were stored at 4° C. until the end of the experiment and centrifuged at 4,000 rpm for 10 minutes. The supernatant were diluted 1:1 with deionized water and analyzed using LC-MS. Percentage remaining at each time point was calculated based on the peak area ratio (analyte over internal standard) relative to the initial level at time zero. Half-lives were calculated by fitting to a first-order exponential decay equation using GraphPad.

### Example 13

### Modified Experimental for Peptides Prone to “Non-Specific Binding”

Compounds of interest (at concentration of 20 μM) were mixed with pre-warmed FaSSIF (1% pancreatin in final working solution). The solution mixture was aliquoted and incubated at 37° C. The number of aliquots required was equivalent to the number of time points (e.g. 0, 0.25, 1, 3, 6 and 24 hr). At each time point, one aliquot was taken and immediately quenched with 4 volumes of organic solvent (acetonitrile/methanol (1:1) and 0.10% formic acid, containing 1 μM internal standard). The remaining steps were the same as the generic experimental.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

At least some of the chemical names or sequences of compounds of the invention as given and set forth in this application, may have been generated on an automated basis by use of a commercially available chemical naming software program, and have not been independently verified. In the instance where the indicated chemical name or sequence and the depicted structure differ, the depicted structure will control. In the chemical structures where a chiral center exists in a structure but no specific stereochemistry is shown for the chiral center, both enantiomers associated with the chiral structure are encompassed by the structure. Similarly, for the peptides where E/Z isomers exist but are not specifically mentioned, both isomers are specifically disclosed and covered.

From the foregoing it will be appreciated that, although specific embodiments of the invention have been described herein for purposes of illustration, various modifications may be made without deviating from the spirit and scope of the invention.

1. A hepcidin analogue comprising a peptide according to Formula W-I:
R¹-X1-Thr-His-X4-B2-X6-X7-Xaa1-X9-Xaa2-J-Y1-Y2-R² (W-I)
or a peptide dimer comprising two peptides according to Formula W-I;
or a pharmaceutically acceptable salt, or a solvate thereof,
wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is —NH₂ or —OH;
X1 is Asp, (D)Asp, or substituted Asp;
X4 is F, NMePhe, or Dpa;
i) Xaa1 is B5; B5 is absent, Lys, D-Lys, Spiro_pip, Arg(nitro), Arg(dimethyl), Cit, Pro(4-amino), or Cav; Xaa2 is B7(L1Z); and B7 is Lys, or D-Lys;
or
ii) Xaa1 is B5(L1Z); B5 is Lys, D-Lys, NMeLys, Dap or Dap-Dap; Xaa2 is B7; and B7 is Glu, NMeGlu, or absent;
B2 is Pro, hydroxyPro, hydroxy(D)Pro, or NPC;
X6 is Cys, NMeCys, SMeCys, or aMeCys;
X7 is Ile, Leu, or NMeLeu;
X9 is Phe, bhPhe, or NMePhe;
L1 is absent, Dap, D-Dap,-eF isoGlu, PEG, Ahx, isoGlu-PEG, PEG-isoGlu, PEG-Ahx, isoGlu-Ahx, isoGlu-PEG-Ahx, or PEG-pep;
Ahx is an aminohexanoic acid moiety; pep is a peptide consisting of one or more amino acids;
PEG is —[C(O)—CH₂—(Peg)ₙ-N(H)]ₘ—, or —[C(O)—CH₂—CH₂—(Peg)ₙ-N(H)]ₘ—; and Peg is —OCH₂CH₂—, m is 1, 2, or 3; and n is an integer between 1-100K;
Z is a half-life extension moiety;
J is absent, any amino acid, or a peptide chain consisting of 1-5 amino acids, wherein each amino acid is independently selected from Pro, (D)Pro, hydroxyPro, hydroxy(D)Pro, Arg, MeArg, Lys, (D)Lys, Lys(Ac), (D)Lys(Ac), Ser, MeSer, Sar, and Gly;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
hydroxyPro is Proline substituted with hydroxy, hydroxy(D)Pro is (D)Proline substituted with hydroxy,
wherein
i) the peptide of formula W-I is optionally PEGylated on one or more R¹, B5, B6, B7, J, Y2, or R²; and
ii) the peptide is optionally cyclized via a disulfide bond between X6 and Y1;
or the hepcidin analogue is any one of peptides listed in Table 3A or Table 3B, and wherein the peptide is optionally cyclized via a disulfide bond between X6 and Y1.

2. The hepcidin analogue comprising a peptide according to claim 1, wherein:
(a) X1 is Asp,
X4 is Dpa;
Xaa1 is B5(L1Z): B5 is Lys, D-Lys, Dap or Dap-Dap; and Xaa2 is B7; and B7 is Glu or absent;
(b) Xaa1 is B5; B5 is absent, Lys, or D-Lys; and Xaa2 is B7(L1Z); and B7 is Lys, or D-Lys:
B2 is Pro or NPC:
X7 is Ile;
X9 is Phe or bhPhe: or
(c) Xaa1 is B5; B5 is absent, Lys, or D-Lys; and Xaa2 is B7(L1Z); and B7 is Lys, or D-Lys.

3-7. (canceled)

8. The hepcidin analogue comprising a peptide according to claim 1, wherein:
(a) J is absent, any amino acid, or a peptide chain consisting of 1-5 amino acids, wherein each amino acid is independently selected from Pro, (D)Pro, hydroxyPro, hydroxy(D)Pro, Arg, MeArg, Lys, (D)Lys, Lys(Ac), (D)Lys(Ac), Ser, MeSer, Sar, and Gly;
(b) J is Arg, Lys, D-Lys, Spiro_pip, Arg(nitro), Arg(dimethyl), Cit, Pro(4-amino), Cav, Pro-, Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly- (SEQ ID NO:251), -Pro-Lys(Ac)-, -Pro-(D)Lys(Ac)-, -Pro-Arg-Ser-Lys(Ac)-(SEQ ID NO:249), -Pro-Arg-Ser-Lys(Ac)-Sar-(SEQ ID NO:250), -Pro-Arg-Ser-Lys(Ac)-Gly-, -HydroxyPro-Arg-Ser-Lys-Gly-(SEQ ID NO:251), -Pro-MeArg-Ser-Lys-Gly-, -Pro-Arg-MeSer-Lys-Gly- (SEQ ID NO:251), (SEQ ID NO:251), -Pro-Lys(Ac)-Ser-Lys(Ac)-, -Pro-Lys(Ac)-Ser-Lys(Ac)-Gly-, -Pro-Lys(Ac)-Ser-Lys(Ac)-Gly-, -Pro-Lys(Ac)-Ser-Lys(Ac)-Sar-, -Pro-Arg-Ser-MeLys-Gly-, or absent; or J is any amino acid; or
(c) J is Arg, Lys, D-Lys, Spiro_pip, Arg(nitro), Arg(dimethyl), Cit, Pro(4-amino), Cav, Pro-, Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly- (SEQ ID NO:251), or absent; or J is any amino acid.

9.-10. (canceled)

11. The hepcidin analogue of claim 1, comprising a peptide according to Formula I:
R¹-Asp-Thr-His-Dpa-B2-Cys-Ile-Xaa1-bhF-Xaa2-J-Y1-Y2-R² (I)
or a peptide dimer comprising two peptides according to Formula I, or a pharmaceutically acceptable salt, or a solvate thereof,
wherein:
R¹ is hydrogen, C₁-C₆ alkyl, C₆-C₁₂ aryl, C₆-C₁₂ aryl-C₁-C₆ alkyl, C₁-C₂₀ alkanoyl, or C₁-C₂₀ cycloalkanoyl;
R² is —NH₂ or —OH;
i) Xaa1 is B5; B5 is absent, Lys, D-Lys, Spiro, pip, Arg(nitro), Arg(dimethyl), Cit, Pro(4-amino), or Cav; Xaa2 is B7(L1Z); and B7 is Lys or D-Lys;
or
ii) Xaa1 is B5(L1Z); B5 is Lys, D-Lys, Dap or Dap-Dap; Xaa2 is B7; and B7 is Glu or absent;
B2 is Pro or NPC;
B7 is Lys or D-Lys;
L1 is absent, Dap, D-Dap, isoGlu, PEG, Ahx, isoGlu-PEG, PEG-isoGlu, PEG-Ahx, isoGlu-Ahx, isoGlu-PEG-Ahx, or PEG-pep;
Ahx is an aminohexanoic acid moiety; pep is a peptide consisting of one or more amino acids;
PEG is —[C(O)—CH₂—(Peg)ₙ-N(H)]ₘ—, or —[C(O)—CH₂—CH₂—(Peg)ₙ-N(H)]ₘ—; and Peg is —OCH₂CH₂—, m is 1, 2, or 3; and n is an integer between 1-100K;
Z is a half-life extension moiety;
J is Arg, Lys, D-Lys, Spiro_pip, Arg(nitro), Arg(dimethyl), Cit, Pro(4-amino), Cav, Pro, -Pro-Arg-, -Pro-Lys-, -Pro-(D)Lys-, -Pro-Arg-Ser-, -Pro-Arg-Ser-Lys-(SEQ ID NO:249), -Pro-Arg-Ser-Lys-Sar-(SEQ ID NO:250), -Pro-Arg-Ser-Lys-Gly- (SEQ ID NO:251), or absent; or J is any amino acid;
Y1 is Cys, homoCys, (D)Cys, NMeCys, aMeCys, or Pen;
Y2 is an amino acid or absent;
Dap is diaminopropanoic acid, Dpa or DIP is 3,3-diphenylalanine or b,b-diphenylalanine, bhPhe is b-homophenylalanine, Bip is biphenylalanine, bhPro is b-homoproline, Tic is L-1,2,3,4,-tetrahydro-isoquinoline-3-carboxylic acid, NPC is L-nipecotic acid, bhTrp is b-homoTryptophane, 1-Nal is 1-naphthylalanine, 2-Nal is 2-naphthylalanine, Orn is orinithine, Nleu is norleucine, Abu is 2-aminobutyric acid, Pen is penicillamine;
substituted Phe is phenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted bhPhe is b-homophenylalanine wherein phenyl is substituted with F, Cl, Br, I, OH, methoxy, dimethoxy, dichloro, dimethyl, difluoro, pentafluoro, allyloxy, azido, nitro, 4-carbamoyl-2,6-dimethyl, trifluoromethoxy, trifluoromethyl, phenoxy, benzyloxy, carbamoyl, t-Bu, carboxyl, CN, or guanidine;
substituted Trp is N-methyl-L-tryptophan, a-methyltryptophan, or tryptophan substituted with F, Cl, OH, or t-Bu;
substituted bhTrp is N-methyl-L-b-homotryptophan, a-methyl-b-homotryptophan, or b-homotryptophan substituted with F, Cl, OH, or t-Bu;
wherein
i) the peptide of formula I is optionally PEGylated on one or more R¹, B5, B6, B7, J, Y2, or R²; and
ii) the peptide is optionally cyclized via a disulfide bond between Cys and Y1.

12.-14. (canceled)

15. The hepcidin analogue according to claim 1, comprising:
(a) a peptide according to Formula IIa:
R¹-Asp-Thr-His-Dpa-Pro-Cys-Ile-B5-bhF-B7(L1Z)-J-Y1-Y2-R² (IIa)
or a peptide dimer comprising two peptides according to Formula IIa, or a pharmaceutically acceptable salt, or a solvate thereof; or
(b) a peptide according to Formula IIb:
R¹-Asp-Thr-His-Dpa-Pro-Cys-Ile-B5(L1Z)-bhF-B7-J-Y1-Y2-R² (IIb)
or a peptide dimer comprising two peptides according to Formula IIa or Formula IIb, or a pharmaceutically acceptable salt, or a solvate thereof,
wherein R¹, B5, B7, L1, Z, J, Y1, Y2 and R² are as described in claim 1.

16. (canceled)

17. The hepcidin analogue according to claim 1,
wherein:
(a) B7(L1Z) is —N(H)C[CH₂(CH₂CH₂CH₂)ₘN(H)L1Z](H)—C(O)—; and wherein m is 0 or 1;
(b) B7(L1Z) is —N(H)C[CH₂N(H)L1Z](H)—C(O)—;
(c) B7(L1Z) is —N(H)C[CH₂CH₂CH₂CH₂N(H)L1Z](H)—C(O)—:
(d) B5(L1Z) is —N(H)C[CH₂(CH₂CH₂CH₂)ₘN(H)L1Z](H)—C(O)—; and wherein m is 0 or 1:
(e) B5(L1Z) is —N(H)C[CH₂N(H)L1Z](H)—C(O)—;
(f) B5(L1Z) is —N(H)C[CH₂CH₂CH₂CH₂N(H)L1Z](H)—C(O)—; or
(g) B5 is (D)Lys.

18-23. (canceled)

24. The hepcidin analogue according to claim 1, comprising a peptide according to formula IIIa or IIIb:
R¹-Asp-Thr-His-Dpa-Pro-Cys-Ile-(D)Lys-bhF—N(H)C[CH₂N(H)L1Z](H)—C(O)-J-Y1-Y2-R² (IIIa), or
R¹-Asp-Thr-His-Dpa-Pro-Cys-Ile-(D)Lys-bhF—N(H)C[CH₂CH₂CH₂CH₂N(H)L1Z](H)—C(O)-J-Y1-Y2-R² (IIIb)
or a peptide dimer thereof, or a pharmaceutically acceptable salt thereof, wherein R¹, R², L1, Z, J, Y1, and Y2 are as in claim 1.

25. The hepcidin analogue according to claim 1,
wherein:
(a) -J-Y1-Y2- is -Cys-, -Pro-Cys-, -Lys-Cys-, -(D)Lys-Cys-, -Dap-Cys-, -Cys-(D)Lys-, -Dap-hCys-, -Pro-Arg-Cys-, -Pro-Arg-Ser-Cys-(SEQ ID NO:253), -Pro-Arg-Ser-Lys-Cys-(SEQ ID NO:254), or -Pro-Arg-Ser-Lys-Sar-Cys-(SEQ ID NO:255);
(b) -J-Y1-Y2- is -(D)Lys-Cys- or -Lys-Cys-:
(c) -J-Y1-Y2- is -Cys-(D)Lys-;
(d) -J-Y1-Y2- is -Pro-Arg-Ser-Lys-Cys-(SEQ ID NO:254):
(e) -J-Y1-Y2- is -Pro-Arg-Ser-Lys-Cys-Lys-(SEQ ID NO:255);
(f) -J-Y1-Y2- is -Pro-Cys-,
(g) -J-Y1-Y2- is -Cys-; or
(h) -J-Y1-Y2- is -(D)Lys-Pen-.

26.-32. (canceled)

33. The hepcidin analogue according to claim 1,
wherein:
(a) L1 is a single bond,
(b) L1 is iso-Glu:
(c) L1 is Ahx;
(d) L1 is iso-Glu-Ahx;
(e) L1 is PEG;
(f) L1 is PEG-Ahx; or
(g) iso-Glu-PEG-Ahx.

34.-39. (canceled)

40. The hepcidin analogue according to claim 1,
wherein:
PEG is —[C(O)—CH2-(Peg)n-N(H)]m or —[C(O)—CH2—CH2-(Peg)n —N(H)]m, and Peg is —OCH2CH2—, m is 1, 2, or 3; and n is an integer between 1-100, or is 10K, 20K, or 30K;
PEG is 1Peg2, and 1Peg2 is —C(O)—CH2-(Peg)2-N(H)—:
PEG is 2Peg2, and 2Peg2 is —C(O)—CH2—CH2-(Peg)2-N(H)—:
PEG is 1Peg2-1Peg2, and each 1Peg2 is —C(O)—CH2—CH2-(Peg)2-N(H)—:
PEG is 1Peg2-1Peg2, and 1Peg2-1Peg2 is —[(C(O)—CH2—(OCH2CH2)2—NH—C(O)—CH2—(OCH2CH2)2—NH—]—,
PEG is 2Peg4, and 2Peg4 is —C(O)—CH2—CH2-(Peg)4-N(H)— or —[C(O)—CH2—CH2—(OCH2CH2)4—NH]—;
PEG is 1Peg8, and 1Peg8 is —C(O)—CH2—(Peg)8-N(H)— or —[C(O)—CH2—(OCH2CH2)8—NH]—;
PEG is 2Peg8, and 2Peg8 is —C(O)—CH2—CH2—(Peg)8-N(H)— or —[C(O)—CH2—CH2—(OCH2CH2)8—NH]—;
PEG is 1Peg11;, and 1Peg11 is —C(O)—CH2—(Peg)11-N(H)— or —[C(O)—CH2—(OCH2CH2)11-NH]—;
PEG is 2Peg11, and 2Peg11 is —C(O)—CH2—CH2—(Peg)11-N(H)— or —[C(O)—CH2—CH2—(OCH2CH2)11—NH]—; or
PEG is 2Peg11′ or 2Peg12, and 2Peg11′ or 2Peg12 is —C(O)—CH2—CH2—(Peg)12-N(H)— or —[C(O)—CH2—CH2—(OCH2CH2)12-NH]—.

41.-58. (canceled)

59. The hepcidin analogue according to claim 1,
wherein:
when PEG is attached to Lys, the —C(O)— of PEG is attached to Ne of Lys;
when PEG is attached to isoGlu, the —N(H)— of PEG is attached to —C(O)— of isoGlu;
when PEG is attached to Ahx, the —N(H)— of PEG is attached to —C(O)— of Ahx or
when PEG is attached to Palm, the —N(H)— of PEG is attached to —C(O)— of Palm.

60.-62. (canceled)

63. The hepcidin analogue according to claim 1, wherein Z is Palm.

64. The hepcidin analogue according to claim 1, wherein R² is NH₂.

65. (canceled)

66. A hepcidin analogue comprising or consisting of a peptide or a dimer thereof, wherein the peptide comprises or consists of any one of the peptides listed in Table 3A or Table 3B; wherein the peptide is cyclized via a disulfide bond between two Cys, Cys and N-MeCys, or Cys and Pen, and wherein * represents that Peg11 is Peg11-OMe.

67.-69. (canceled)

70. A pharmaceutical composition comprising the hepcidin analogue or dimer of claim 1, and a pharmaceutically acceptable carrier, excipient or vehicle.

71. A method of binding a ferroportin or inducing ferroportin internalization and degradation, comprising contacting the ferroportin with at least one hepcidin analogue or dimer of claim 1, or the composition of claim 70.

72.-79. (canceled)


SEQUENCE LISTING (360 sequences)

SEQ ID NO: 1 - protein, 11 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Dap_Dap_Palm
  MOD_RES (10)..(10): bhPhe
XDTHXPCIXXC

SEQ ID NO: 2 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
XDTHXPCIXXXRC

SEQ ID NO: 3 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): (D)Lys
  MOD_RES (13)..(13): N-MeCys
XDTHXPCIXXXXX

SEQ ID NO: 4 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): bhPhe
  MOD_RES (11)..(11): Lys(Ahx_Palm)
  MOD_RES (12)..(12): (D)Lys
XDTHXXCIXXXXC

SEQ ID NO: 5 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys_Ahx_IsoGlu_Palm
  MOD_RES (10)..(10): bhPhe
  MOD_RES (11)..(11): (D)Lys
XDTHXPCIXXXC

SEQ ID NO: 6 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys(IsoGlu_palm)
  MOD_RES (10)..(10): bhPhe
  MOD_RES (11)..(11): (D)Lys
XDTHXPCIXXXC

SEQ ID NO: 7 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_Ahx_Behenic_Acid
  MOD_RES (12)..(12): (D)Lys
XDTHXPCIXXXXC

SEQ ID NO: 8 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys_Ahx_Behenic_Acid
  MOD_RES (10)..(10): bhPhe
  MOD_RES (11)..(11): (D)Lys
XDTHXPCIXXXC

SEQ ID NO: 9 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys_1PEG2_1PEG2_IsoGlu_Behenic_Acid
  MOD_RES (10)..(10): bhPhe
  MOD_RES (11)..(11): (D)Lys
XDTHXPCIXXXC

SEQ ID NO: 10 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys(1PEG2_1PEG2_Ahx_C18_Diacid)
  MOD_RES (10)..(10): bhPhe
  MOD_RES (12)..(12): (D)Lys
XDTHXPCIXXKXC

SEQ ID NO: 11 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys(1PEG2_1PEG2_Ahx_C18_Diacid)
  MOD_RES (10)..(10): bhPhe
  MOD_RES (11)..(11): (D)Lys
XDTHXPCIXXXC

SEQ ID NO: 12 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): bhPhe
  MOD_RES (11)..(11): Lys(Ahx_Palm)
  MOD_RES (13)..(13): (D)Lys
XDTHXXCIXXXCX

SEQ ID NO: 13 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): bhPhe
  MOD_RES (11)..(11): Lys(IsoGlu_palm)
  MOD_RES (13)..(13): (D)Lys
XDTHXXCIXXXCX

SEQ ID NO: 14 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): bhPhe
  MOD_RES (11)..(11): Lys_Ahx_IsoGlu_Palm
  MOD_RES (13)..(13): (D)Lys
XDTHXXCIXXXCX

SEQ ID NO: 15 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): bhPhe
  MOD_RES (11)..(11): Lys(1PEG2_1PEG2_Ahx_C18_Diacid)
  MOD_RES (13)..(13): (D)Lys
XDTHXXCIXXXCX

SEQ ID NO: 16 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_Palm
XDTHXPCIXXXKC

SEQ ID NO: 17 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
XDTHXPCIXXXKC

SEQ ID NO: 18 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): bhPhe
  MOD_RES (10)..(10): Lys(Ahx_Palm)
  MOD_RES (11)..(11): (D)Lys
XDTHXPCIXXXC

SEQ ID NO: 19 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): Lys(IsoGlu_palm)
  MOD_RES (10)..(10): bhPhe
  MOD_RES (12)..(12): (D)Lys
XDTHXXCIXXCX

SEQ ID NO: 20 - protein, 13 aa, Artificial Sequence
  Made in Lab - peptide analogs of hepcidin
  MOD_RES (1)..(1): Isovaleric Acid
  MOD_RES (2)..(2): IsoAsp
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): (D)Lys
XXTHXPCIXXXXC

SEQ ID NO: 21 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Spiral_Pip
XDTHXPCIXXXXC

SEQ ID NO: 22 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Arg_Nitro
XDTHXPCIXXXXC

SEQ ID NO: 23 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Arg_Dimethyl
XDTHXPCIXXXXC

SEQ ID NO: 24 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Cit
XDTHXPCIXXXXC

SEQ ID NO: 25 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Pro_4Amino
XDTHXPCIXXXXC

SEQ ID NO: 26 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ahx_Palm)
  MOD_RES (12)..(12): Cav
XDTHXPCIXXXXC

SEQ ID NO: 27 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Spiral_Pip
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 28 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Arg_Nitro
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 29 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Arg_Dimethyl
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 30 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Cit
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 31 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Pro_4Amino
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 32 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Cav
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 33 - protein, 16 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_Palm)
XDTHXPCIXXXPRSKC

SEQ ID NO: 34 - protein, 17 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_Palm)
  MOD_RES (16)..(16): Sarc
XDTHXPCIXXXPRSKXC

SEQ ID NO: 35 - protein, 18 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_Palm)
  MOD_RES (13)..(13): (D)Lys
  MOD_RES (15)..(15): (D)Lys
  MOD_RES (16)..(16): Sarc
  MOD_RES (18)..(18): (D)Lys
XDTHXPCIXXXPXSXXCX

SEQ ID NO: 36 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_PEG12_Sarc_Palm
  MOD_RES (12)..(12): (D)Lys
XDTHXPCIXXXXC

SEQ ID NO: 37 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_Ahx_Palm)
  MOD_RES (12)..(12): (D)Lys
XDTHXPCIXXXXC

SEQ ID NO: 38 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_Lys[PEG12_Palm]2
  MOD_RES (12)..(12): (D)Lys
XDTHXPCIXXXXC

SEQ ID NO: 39 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_Ahx_Ahx_Palm
  MOD_RES (12)..(12): (D)Lys
XDTHXPCIXXXXC

SEQ ID NO: 40 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_Ahx_Ahx_Ahx_Palm
  MOD_RES (12)..(12): (D)Lys
XDTHXPCIXXXXC

SEQ ID NO: 41 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_PEG12_Palm)
  MOD_RES (12)..(12): (D)Lys
XDTHXPCIXXXXC

SEQ ID NO: 42 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(1PEG2_1PEG2_IsoGlu_C18_Diacid)
  MOD_RES (12)..(12): (D)Lys
XDTHXPCIXXXXC

SEQ ID NO: 43 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(1PEG2_1PEG2_Ahx_C18_Diacid)
  MOD_RES (12)..(12): (D)Lys
XDTHXPCIXXXXC

SEQ ID NO: 44 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_KKK_Palm)
  MOD_RES (12)..(12): (D)Lys
XDTHXPCIXXXXC

SEQ ID NO: 45 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys_PEG12_KKKGS_Palm
  MOD_RES (12)..(12): (D)Lys
XDTHXPCIXXXXC

SEQ ID NO: 46-200 - skipped (155 entries)

SEQ ID NO: 201 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (8)..(8): meL
  MOD_RES (9)..(9): (D)Lys
  MOD_RES (10)..(10): bhPhe
  MOD_RES (11)..(11): Lys(Ahx_Palm)
  MOD_RES (12)..(12): (D)Lys
XDTHXPCXXXXXC

SEQ ID NO: 202 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): N-MePhe
  MOD_RES (11)..(11): Lys(Ahx_Palm)
  MOD_RES (12)..(12): (D)Lys
XDTHXPCIXXXXC

SEQ ID NO: 203 - protein, 13 aa, Artificial Sequence
  Made in Lab - peptide analogs of hepcidin
  MOD_RES (1)..(1): Isovaleric Acid
  MOD_RES (5)..(5): N-MePhe
  MOD_RES (9)..(9): (D)Lys
  MOD_RES (10)..(10): bhPhe
  MOD_RES (11)..(11): Lys(Ahx_Palm)
  MOD_RES (12)..(12): (D)Lys
XDTHXPCIXXXXC

SEQ ID NO: 204 - protein, 18 aa, Artificial Sequence
  Made in Lab - peptide analogs of hepcidin
  MOD_RES (1)..(1): Isovaleric Acid
  MOD_RES (2)..(2): (D)Asp
  MOD_RES (9)..(9): Lys(IsoGlu_palm)
XXTHFPCIXFEPRSKGCK

SEQ ID NO: 205 - protein, 13 aa, Artificial Sequence
  Made in Lab - peptide analogs of hepcidin
  MOD_RES (1)..(1): Isovaleric Acid
  MOD_RES (9)..(9): (D)Lys
  MOD_RES (10)..(10): bhPhe
  MOD_RES (11)..(11): Lys_Palm
XDTHFPCIXXXKC

SEQ ID NO: 206 - skipped

SEQ ID NO: 207 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (18)..(18): Lys_Alexa647
XDTHFPCIXFEPRSKGCX

SEQ ID NO: 208 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (15)..(15): Lys_PEG12_Biotin
XDTHFPCIXFEPRSXGCK

SEQ ID NO: 209 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (15)..(15): Lys_Ahx_Biotin
XDTHFPCIXFEPRSXGCK

SEQ ID NO: 210 - protein, 18 aa, Artificial Sequence
  Made in Lab - peptide analogs of hepcidin
  MOD_RES (1)..(1): Isovaleric Acid
  MOD_RES (11)..(11): Lys(1PEG2_1PEG2_Ahx_C18_Diacid)
  MOD_RES (16)..(16): Sarc
XDTHFPCIKFXPRSKXCK

SEQ ID NO: 211 - protein, 18 aa, Artificial Sequence
  Made in Lab - peptide analogs of hepcidin
  MOD_RES (1)..(1): Isovaleric Acid
  MOD_RES (11)..(11): Lys_PEG12_Sarc_Palm
  MOD_RES (16)..(16): Sarc
XDTHFPCIKFXPRSKXCK

SEQ ID NO: 212 - protein, 18 aa, Artificial Sequence
  Made in Lab - peptide analogs of hepcidin
  MOD_RES (1)..(1): Isovaleric Acid
  MOD_RES (5)..(5): Dpa
  MOD_RES (11)..(11): Lys(PEG12_Palm)
  MOD_RES (16)..(16): Sarc
XDTHXPCIKFXPRSKXCK

SEQ ID NO: 213 - protein, 18 aa, Artificial Sequence
  Made in Lab - peptide analogs of hepcidin
  MOD_RES (1)..(1): Isovaleric Acid
  MOD_RES (9)..(9): Lys(Ac)
  MOD_RES (11)..(11): Lys(PEG12_Palm)
  MOD_RES (15)..(15): Lys(Ac)
  MOD_RES (16)..(16): Sarc
XDTHFPCIXFXPRSXXCK

SEQ ID NO: 214 - protein, 18 aa, Artificial Sequence
  Made in Lab - peptide analogs of hepcidin
  MOD_RES (1)..(1): Isovaleric Acid
  MOD_RES (9)..(9): Lys(Ac)
  MOD_RES (11)..(11): Lys(PEG12_Palm)
  MOD_RES (13)..(13): Lys(Ac)
  MOD_RES (15)..(15): Lys(Ac)
  MOD_RES (16)..(16): Sarc
XDTHFPCIXFXPXSXXCK

SEQ ID NO: 215 - protein, 18 aa, Artificial Sequence
  Made in Lab - peptide analogs of hepcidin
  MOD_RES (1)..(1): Isovaleric Acid
  MOD_RES (9)..(9): Lys_IsoGlu
XDTHFPCIXFEPRSKGCK

SEQ ID NO: 216 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
XDTHFPCLXFEPRSKGCK

SEQ ID NO: 217 - protein, 18 aa, Artificial Sequence
  Made in Lab - peptide analogs of hepcidin
  MOD_RES (1)..(1): Isovaleric Acid
  MOD_RES (7)..(7): CMe
  MOD_RES (9)..(9): Lys(IsoGlu_palm)
  MOD_RES (17)..(17): CMe
XDTHFPXIXFEPRSKGXK

SEQ ID NO: 218 - protein, 17 aa, Artificial Sequence
  Made in Lab - peptide analogs of hepcidin
  MOD_RES (1)..(1): Isovaleric Acid
  MOD_RES (9)..(9): Lys(IsoGlu_palm)
XDTHFPCIXFEPRSKGC

SEQ ID NO: 219 - protein, 13 aa, Artificial Sequence
  Made in Lab - peptide analogs of hepcidin
  MOD_RES (1)..(1): Isovaleric Acid
  MOD_RES (9)..(9): Lys(IsoGlu_palm)
XDTHFPCIXFEPR

SEQ ID NO: 220 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (18)..(18): Lys(Ac)
XDTHFPCIXFEPRSKGCX

SEQ ID NO: 221 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (15)..(15): Lys(Ac)
XDTHFPCIXFEPRSXGCK

SEQ ID NO: 222 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (13)..(13): Lys(Ac)
XDTHFPCIXFEPXSKGCK

SEQ ID NO: 223 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (15)..(15): Lys(Ac)
  MOD_RES (18)..(18): Lys(Ac)
XDTHFPCIXFEPRSXGCX

SEQ ID NO: 224 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (13)..(13): Lys(Ac)
  MOD_RES (18)..(18): Lys(Ac)
XDTHFPCIXFEPXSKGCX

SEQ ID NO: 225 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (13)..(13): Lys(Ac)
  MOD_RES (15)..(15): Lys(Ac)
XDTHFPCIXFEPXSXGCK

SEQ ID NO: 226 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (13)..(13): Lys(Ac)
  MOD_RES (15)..(15): Lys(Ac)
  MOD_RES (18)..(18): Lys(Ac)
XDTHFPCIXFEPXSXGCX

SEQ ID NO: 227 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
XDTHFPCIXFEPRSKGCK

SEQ ID NO: 228 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (18)..(18): Lys(Ac)
XDTHFPCIXFEPRSKGCX

SEQ ID NO: 229 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (15)..(15): Lys(Ac)
XDTHFPCIXFEPRSXGCK

SEQ ID NO: 230 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (13)..(13): Lys(Ac)
XDTHFPCIXFEPXSKGCK

SEQ ID NO: 231 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (15)..(15): Lys(Ac)
  MOD_RES (18)..(18): Lys(Ac)
XDTHFPCIXFEPRSXGCX

SEQ ID NO: 232 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (13)..(13): Lys(Ac)
  MOD_RES (18)..(18): Lys(Ac)
XDTHFPCIXFEPXSKGCX

SEQ ID NO: 233 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (13)..(13): Lys(Ac)
  MOD_RES (15)..(15): Lys(Ac)
XDTHFPCIXFEPXSXGCK

SEQ ID NO: 234 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_Ac
  MOD_RES (13)..(13): Lys(Ac)
  MOD_RES (15)..(15): Lys(Ac)
  MOD_RES (18)..(18): Lys(Ac)
XDTHFPCIXFEPXSXGCX

SEQ ID NO: 235 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (18)..(18): N-MeLys
XDTHFPCIXFEPRSKGCX

SEQ ID NO: 236 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (17)..(17): N-MeCys
XDTHFPCIXFEPRSKGXK

SEQ ID NO: 237 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (16)..(16): Sarc
XDTHFPCIXFEPRSKXCK

SEQ ID NO: 238 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (15)..(15): N-MeLys
XDTHFPCIXFEPRSXGCK

SEQ ID NO: 239 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (14)..(14): meS
XDTHFPCIXFEPRXKGCK

SEQ ID NO: 240 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (13)..(13): meR
XDTHFPCIXFEPXSKGCK

SEQ ID NO: 241 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (11)..(11): N-MeGlu
XDTHFPCIXFXPRSKGCK

SEQ ID NO: 242 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (10)..(10): N-MePhe
XDTHFPCIXXEPRSKGCK

SEQ ID NO: 243 - protein, 18 aa, Artificial Sequence
  Made in Lab - peptide analogs of hepcidin
  MOD_RES (1)..(1): Isovaleric Acid
  MOD_RES (9)..(9): meLys_IsoGlu_Palm
XDTHFPCIXFEPRSKGCK

SEQ ID NO: 244 - protein, 18 aa, Artificial Sequence
  Made in Lab - peptide analogs of hepcidin
  MOD_RES (1)..(1): Isovaleric Acid
  MOD_RES (8)..(8): meL
  MOD_RES (9)..(9): Lys(IsoGlu_palm)
XDTHFPCXXFEPRSKGCK

SEQ ID NO: 245 - protein, 18 aa, Artificial Sequence
  Made in Lab - peptide analogs of hepcidin
  MOD_RES (1)..(1): Isovaleric Acid
  MOD_RES (7)..(7): N-MeCys
  MOD_RES (9)..(9): Lys(IsoGlu_palm)
XDTHFPXIXFEPRSKGCK

SEQ ID NO: 246 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_PEG12_Biotin
XDTHFPCIXFEPRSKGCK

SEQ ID NO: 247 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_PEG3_Mal
XDTHFPCIXFEPRSKGCK

SEQ ID NO: 248 - protein, 18 aa, Artificial Sequence
  Made in Lab - peptide analogs of hepcidin
  MOD_RES (1)..(1): Isovaleric Acid
  MOD_RES (6)..(6): dHyp_4S
  MOD_RES (9)..(9): Lys(IsoGlu_palm)
XDTHFXCIXFEPRSKGCK

SEQ ID NO: 249 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (12)..(12): dHyp_4S
XDTHFPCIXFEXRSKGCK

SEQ ID NO: 250 - protein, 18 aa, Artificial Sequence
  Made in Lab - peptide analogs of hepcidin
  MOD_RES (1)..(1): Isovaleric Acid
  MOD_RES (6)..(6): Hyp_4S
  MOD_RES (9)..(9): Lys(IsoGlu_palm)
XDTHFXCIXFEPRSKGCK

SEQ ID NO: 251 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (12)..(12): Hyp_4S
XDTHFPCIXFEXRSKGCK

SEQ ID NO: 252 - protein, 18 aa, Artificial Sequence
  Made in Lab - peptide analogs of hepcidin
  MOD_RES (1)..(1): Isovaleric Acid
  MOD_RES (9)..(9): Lys_IsoGlu_HydroxyPalmSR
XDTHFPCIXFEPRSKGCK

SEQ ID NO: 253 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys(PEG12_OMe)
  MOD_RES (10)..(10): bhPhe
  MOD_RES (11)..(11): Lys(1PEG2_1PEG2_IsoGlu_C18_Diacid)
  MOD_RES (12)..(12): (D)Lys(PEG12_OMe)
XDTHXPCIXXXXC

SEQ ID NO: 254 - protein, 18 aa, Artificial Sequence
  Made in Lab - peptide analogs of hepcidin
  MOD_RES (1)..(1): Isovaleric Acid
  MOD_RES (6)..(6): Hyp_3S
  MOD_RES (9)..(9): Lys(IsoGlu_palm)
XDTHFXCIXFEPRSKGCK

SEQ ID NO: 255 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (12)..(12): Hyp_3S
XDTHFPCIXFEXRSKGCK

SEQ ID NO: 256 - protein, 18 aa, Artificial Sequence
  Made in Lab - peptide analogs of hepcidin
  MOD_RES (1)..(1): Isovaleric Acid
  MOD_RES (6)..(6): Hyp_3R
  MOD_RES (9)..(9): Lys(IsoGlu_palm)
XDTHFXCIXFEPRSKGCK

SEQ ID NO: 257 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (12)..(12): Hyp_3R
XDTHFPCIXFEXRSKGCK

SEQ ID NO: 258 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_Palm)_OH
XDTHFPCIXFEPRSKGCK

SEQ ID NO: 259-300 - skipped (42 entries)

SEQ ID NO: 301 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
  MOD_RES (18)..(18): Lys(AFDye647)
XDTHFPCIXFEPRSKGCX

SEQ ID NO: 302 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_16F3Palm)
XDTHFPCIXFEPRSKGCK

SEQ ID NO: 303 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_15OHPalm)
XDTHFPCIXFEPRSKGCK

SEQ ID NO: 304 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_14OHPalm)
XDTHFPCIXFEPRSKGCK

SEQ ID NO: 305 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_12OHPalm)
XDTHFPCIXFEPRSKGCK

SEQ ID NO: 306 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_11OHPalm)
XDTHFPCIXFEPRSKGCK

SEQ ID NO: 307 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_10OHPalm)
XDTHFPCIXFEPRSKGCK

SEQ ID NO: 308 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_9OHPalm)
XDTHFPCIXFEPRSKGCK

SEQ ID NO: 309 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_7OHPalm)
XDTHFPCIXFEPRSKGCK

SEQ ID NO: 310 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_5OHPalm)
XDTHFPCIXFEPRSKGCK

SEQ ID NO: 311 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_4OHPalm)
XDTHFPCIXFEPRSKGCK

SEQ ID NO: 312 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_2OHPalm)
XDTHFPCIXFEPRSKGCK

SEQ ID NO: 313 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_8OHPalm)
XDTHFPCIXFEPRSKGCK

SEQ ID NO: 314 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_13OHPalm)
XDTHFPCIXFEPRSKGCK

SEQ ID NO: 315 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_6OHPalm)
XDTHFPCIXFEPRSKGCK

SEQ ID NO: 316 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_Palm)_OH
  MOD_RES (10)..(10): Phe(13C9_15N)
XDTHFPCIXXEPRSKGCK

SEQ ID NO: 317 - protein, 13 aa, Artificial Sequence
  Made in Lab - peptide analogs of hepcidin
  MOD_RES (1)..(1): Isovaleric Acid
  MOD_RES (7)..(7): Cys(SKGCKam)
  MOD_RES (9)..(9): Lys(IsoGlu_palm)
XDTHFPXIXFEPR

SEQ ID NO: 318 - protein, 16 aa, Artificial Sequence
  Made in Lab - peptide analogs of hepcidin
  MOD_RES (1)..(1): Isovaleric Acid
  MOD_RES (7)..(7): Cys(Ckam)
  MOD_RES (9)..(9): Lys(IsoGlu_palm)
XDTHFPXIXFEPRSKG

SEQ ID NO: 319 - protein, 18 aa, Artificial Sequence
  Made in Lab - peptide analogs of hepcidin
  MOD_RES (1)..(1): Isovaleric Acid
  MOD_RES (9)..(9): Lys(IsoGlu_palm)
XDTHFPCIXFEPRSKGCK

SEQ ID NO: 320 - protein, 11 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys(Ahx_Palm)
  MOD_RES (10)..(10): 2Pal
XDTHXPCIXXC

SEQ ID NO: 321 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): Lys(Ahx_Palm)
  MOD_RES (10)..(10): 2Pal
  MOD_RES (11)..(11): (D)Lys
XDTHXPCIXXXC

SEQ ID NO: 322 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): 2Pal
  MOD_RES (11)..(11): Lys(Ahx_Palm)
  MOD_RES (12)..(12): (D)Lys
XDTHXPCIXXXXC

SEQ ID NO: 323 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): bAla
  MOD_RES (10)..(10): bhPhe
  MOD_RES (11)..(11): (D)Lys
XDTHXPCIXXXC

SEQ ID NO: 324 - protein, 11 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): bhPhe
  MOD_RES (10)..(10): (D)Lys
XDTHXPCIXXC

SEQ ID NO: 325 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys(Ac)
  MOD_RES (10)..(10): bhPhe
  MOD_RES (11)..(11): Lys(Ac)
XDTHXPCIXXXC

SEQ ID NO: 326 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(Ac)
XDTHXPCIXXXC

SEQ ID NO: 327 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (12)..(12): (D)Lys
XDTHXPCIXXKXC

SEQ ID NO: 328 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(4-tBu)
  MOD_RES (11)..(11): (D)Lys
  MOD_RES (13)..(13): Lys(PEG12_Palm)
XDTHXXCIXXXCX

SEQ ID NO: 329 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): PEG2
  MOD_RES (10)..(10): bhPhe
  MOD_RES (11)..(11): Lys(PEG12_Palm)
  MOD_RES (12)..(12): (D)Lys
XDTHXPCIXXXXC

SEQ ID NO: 330 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): bhPhe
  MOD_RES (10)..(10): Lys(PEG12_Palm)
  MOD_RES (11)..(11): (D)Lys
XDTHXPCIXXXC

SEQ ID NO: 331 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (10)..(10): bhPhe
  MOD_RES (11)..(11): Lys(PEG12_Palm)
  MOD_RES (12)..(12): (D)Lys
XDTHXPCIIXXXC

SEQ ID NO: 332 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Ala
  MOD_RES (10)..(10): bhPhe
  MOD_RES (11)..(11): Lys(PEG12_Palm)
  MOD_RES (12)..(12): (D)Lys
XDTHXPCIXXXXC

SEQ ID NO: 333 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): bAla
  MOD_RES (10)..(10): bhPhe
  MOD_RES (11)..(11): Lys(PEG12_Palm)
  MOD_RES (12)..(12): (D)Lys
XDTHXPCIXXXXC

SEQ ID NO: 334 - protein, 13 aa, Artificial Sequence
  Made in Lab - peptide analogs of hepcidin
  MOD_RES (1)..(1): Isovaleric Acid
  MOD_RES (9)..(9): (D)Lys
  MOD_RES (11)..(11): Lys(PEG12_Palm)
XDTHFPCIXFXCK

SEQ ID NO: 335 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): Dap
  MOD_RES (13)..(13): Hcy
XDTHXPCIXFXXX

SEQ ID NO: 336 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): Dap
  MOD_RES (13)..(13): Pen
XDTHXPCIXFXXX

SEQ ID NO: 337 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): (D)Lys
XDTHXPCIXFXXC

SEQ ID NO: 338 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): bhLys
XDTHXPCIXFXXC

SEQ ID NO: 339 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): Dab
XDTHXPCIXFXXC

SEQ ID NO: 340 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): Orn
XDTHXPCIXFXXC

SEQ ID NO: 341 - protein, 12 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
XDTHXPCIXFXC

SEQ ID NO: 342 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): (D)Lys
  MOD_RES (13)..(13): N-MeCys
XDTHXPCIXFXXX

SEQ ID NO: 343 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (13)..(13): N-MeCys
XDTHXPCIXFXKX

SEQ ID NO: 344 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (11)..(11): Lys(PEG12_Palm)
  MOD_RES (12)..(12): (D)Lys
  MOD_RES (13)..(13): Pen
XDTHXPCIXFXXX

SEQ ID NO: 345 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): Dpa
  MOD_RES (11)..(11): Lys(PEG12_Palm)
  MOD_RES (12)..(12): (D)Lys
XDTHXPCIXXXXC

SEQ ID NO: 346 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(3,4-diF)
  MOD_RES (11)..(11): Lys(PEG12_Palm)
XDTHXXCIXXXKC

SEQ ID NO: 347 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(4-CN)
  MOD_RES (11)..(11): Lys(PEG12_Palm)
XDTHXXCIXXXKC

SEQ ID NO: 348 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): aMePhe
  MOD_RES (11)..(11): Lys(PEG12_Palm)
XDTHXXCIXXXKC

SEQ ID NO: 349 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (9)..(9): (D)Lys
  MOD_RES (10)..(10): bhPhe
  MOD_RES (11)..(11): Lys(PEG12_Palm)
  MOD_RES (12)..(12): (D)Lys
XDTHXPCIXXXXC

SEQ ID NO: 350 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): bhPro
  MOD_RES (9)..(9): (D)Lys
  MOD_RES (11)..(11): Lys(PEG12_Palm)
  MOD_RES (12)..(12): (D)Lys
XDTHXXCIXFXXC

SEQ ID NO: 351 - protein, 13 aa, Artificial Sequence
  Made in Lab - peptide analogs of hepcidin
  MOD_RES (1)..(1): Isovaleric Acid
  MOD_RES (6)..(6): Npc
  MOD_RES (9)..(9): (D)Lys
  MOD_RES (11)..(11): Lys(PEG12_Palm)
  MOD_RES (12)..(12): (D)Lys
XDTHFXCIXFXXC

SEQ ID NO: 352 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (11)..(11): Lys(PEG12_Palm)
  MOD_RES (12)..(12): (D)Lys
XDTHXXCIXFXXC

SEQ ID NO: 353 - protein, 13 aa, Artificial Sequence
  Made in Lab - peptide analogs of hepcidin
  MOD_RES (1)..(1): Isovaleric Acid
  MOD_RES (7)..(7): aMeCys
  MOD_RES (9)..(9): (D)Lys
  MOD_RES (11)..(11): Lys(PEG12_Palm)
XDTHFPXIXFXKC

SEQ ID NO: 354 - protein, 13 aa, Artificial Sequence
  Made in Lab - peptide analogs of hepcidin
  MOD_RES (1)..(1): Isovaleric Acid
  MOD_RES (7)..(7): aMeCys
  MOD_RES (9)..(9): (D)Lys
  MOD_RES (11)..(11): Lys(PEG12_Palm)
  MOD_RES (13)..(13): aMeCys
XDTHFPXIXFXKX

SEQ ID NO: 355 - protein, 13 aa, Artificial Sequence
  Made in Lab - peptide analogs of hepcidin
  MOD_RES (1)..(1): Isovaleric Acid
  MOD_RES (9)..(9): (D)Lys
  MOD_RES (11)..(11): Lys(PEG12_Palm)
  MOD_RES (13)..(13): aMeCys
XDTHFPCIXFXKX

SEQ ID NO: 356 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(4-COOH)
  MOD_RES (11)..(11): Lys(PEG12_Palm)
XDTHXXCIXXXKC

SEQ ID NO: 357 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): 2Nal
  MOD_RES (11)..(11): Lys(PEG12_Palm)
XDTHXXCIXXXKC

SEQ ID NO: 358 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(4-(2-aminoethoxy)
  MOD_RES (11)..(11): Lys(PEG12_Palm)
XDTHXXCIXXXKC

SEQ ID NO: 359 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (10)..(10): Phe(4-tBu)
  MOD_RES (11)..(11): Lys(PEG12_Palm)
XDTHXXCIXXXKC

SEQ ID NO: 360 - protein, 13 aa, Artificial Sequence
  Made in Lab - peptide analogs of hepcidin
  MOD_RES (1)..(1): Isovaleric Acid
  MOD_RES (5)..(5): Dpa
  MOD_RES (6)..(6): Npc
  MOD_RES (9)..(9): (D)Lys
  MOD_RES (11)..(11): Lys(PEG12_Palm)
XDTHXXCIXFXKC